(19) United States
(12) Patent Application Publication
(10) Pub. No.: US 2017/0260520 A1
(43) Pub. Date: Sep. 14, 2017

(54) Recombinase-Mediated Integration Of A Polynucleotide Library

(71) Applicant: Novozymes A/S, Bagsvaerd (DK)

(72) Inventors: Tomoko Matsui, Chiba (JP); Hiroaki Udagawa, Chiba (JP); Siik Kishishita, Bagsvaerd (DK); Dominique Aubert Skovlund, Bagsvaerd (DK); Qiming Jin, Davis, CA (US)

(21) Appl. No.: 15/505,196

(22) PCT Filed: Aug. 20, 2015
(86) PCT No.: PCT/EP2015/069177

§ 371 Date: Feb. 20, 2017

(30) Foreign Application Priority Data
Aug. 20, 2014 (EP) 14181694.2

(51) Int. Cl. C12N 15/10; C12P 21/00

(52) U.S. Cl. CPC C12N 15/1082; C12P 21/00

(57) ABSTRACT

The present invention provides methods for integrating a polynucleotide library of interest in the chromosome of a filamentous fungal host cell using a site-specific recombinase, polynucleotide library expression systems comprising a host cell and a polynucleotide construct, as well as the resulting filamentous fungal host cells comprising a polynucleotide library and their cultivation to produce a polypeptide.

### REFERENCE TO A SEQUENCE LISTING

This application contains a Sequence Listing in computer readable form, which is incorporated herein by reference.

### FIELD OF THE INVENTION

The present invention relates to methods for integrating a polynucleotide library of interest in the chromosome of a filamentous fungal host cell using a site-specific recombinase, polynucleotide library expression systems comprising a host cell and a polynucleotide construct, as well as the resulting filamentous fungal host cells comprising a polynucleotide library and their cultivation to produce a polypeptide.

### BACKGROUND OF THE INVENTION

A large number of naturally-occurring organisms have been found to produce useful polypeptide products, e.g., enzymes, the large scale production of which is desirable for research and commercial purposes.

Construction of host cells has been described, wherein a highly expressed chromosomal gene is replaced with a recognition sequence of a site-specific recombinase to allow subsequent insertion of a single product-encoding polynucleotide into that site by the use of a recombinase recognizing said sequence (EP 1 405 908 A1; ProBioGen AG).

It has been disclosed to insert DNA at a known location in the genome by making use of site-specific recombination systems that are freely reversible. These reversible systems include the following: the Cre-lox system from bacteriophage P1; the FLP-FRT system of Saccharomyces cerevisiae; the R-RS system of Zygosaccharonzyces rouxii; a modified Gin-gix system from bacteriophage Mu; the beta-recombinase-six system from a Bacillus subtilis plasmid and the delta-gamma-res system from the bacterial transposon Tn1000. Cre, FLP, R, Gin, beta-recombinase and gamma-delta are the recombinases, and lox, FRT, RS, gix, six and res are the respective recombination sites (reviewed by Sadowslu, 1993 FASEB J., 7:750-67; Ow and Medberry, 1995 Crit. Rev. Plant Sci. 14: 239-261).

The site-specific recombination systems above have in common the property that a single polypeptide recombinase catalyzes the recombination between two recognition sites of identical or nearly identical sequences.

When cloning and screening polynucleotides encoding polypeptides of interest in a fungal host cell, especially when screening polynucleotide libraries encoding variants of the same secreted polypeptide of interest for an improved property, it is desirable that the fungal host cells express the polynucleotides at a comparable level, so that their properties can be directly assayed without having to perform a normalization step first. This problem has been addressed, e.g., in EP 1124949, by using cloning and expression vectors comprising a fungal replication initiation sequence, such as, the well-known AMA1 sequence.

### SUMMARY OF THE INVENTION

We provide herein an effective way of quickly and site-specifically inserting a polynucleotide library encoding polypeptides of interest in the chromosome of a filamentous fungal host, thereby achieving highly uniform expression levels in the transformants due to the presence of the inserted genes in just one single copy in the exact same genomic position in each transformed and selected host cell.

The resulting selected transformed cell may even be employed directly to produce the encoded selected polypeptide with improved up-scaled expression levels that correspond to those of the initial screening.

Accordingly, in a first aspect, the present invention relates to methods for integrating a polynucleotide library of interest in the chromosome of a filamentous fungal host cell using a site-specific recombinase, said method comprising the steps of:
a) providing a filamentous fungal host cell comprising in its chromosome in the following order:
   i) a first recognition sequence of the recombinase or a region that is 5′ or 3′ of an integration site;
   ii) a first selection marker;
   iii) a second recognition sequence of the recombinase; and optionally
   iv) a non-functional partial second selection marker;
b) transforming said host cell with a nucleic acid construct comprising in the following order:
   i) the first recognition sequence of the recombinase or the region that is 5′ or 3′ of the integration site;
   ii) a polynucleotide library of interest;
   iii) a second selection marker OR a non-functional partial second selection marker if the corresponding but optional non-functional second selection marker of step (a)(iv) is comprised in the host cell chromosome; and
   iv) the second recognition sequence of the recombinase;
c) expressing a gene encoding the site-specific recombinase in said host cell; and
d) selecting a transformed host cell which expresses the second selection marker and not the first selection marker,

wherein the polynucleotide library of interest is site-specifically integrated in the correct orientation in the chromosome of the host cell by the recombinase, whereby the first selectable marker is excised from the chromosome and whereby any non-functional partial second selectable markers are recombined to form a functional second selection marker in the chromosome.

In a second aspect, the invention relates to polynucleotide library expression systems comprising:

a) a filamentous fungal host cell comprising in its chromosome in the following order:
i) a first recognition sequence of a site-specific recombinase or a region that is 5′ or 3′ of an integration site;
ii) a first selection marker;
iii) a second recognition sequence of the recombinase; and optionally
iv) a non-functional partial second selection marker; AND
   b) a nucleic acid construct comprising in the following elements in order:
i) the first recognition sequence of the recombinase or the region that is 5′ or 3′ of the integration site;
ii) a polynucleotide library of interest;
iii) a second selection marker OR a non-functional partial second selection marker if the optional non-functional second selection marker of step (a)(iv) is comprised in the host cell chromosome; and
iv) the second recognition sequence of the recombinase, and
   c) a gene encoding the site-specific recombinase comprised in the filamentous fungal host cell; preferably in the chromosome, in a second nucleic acid construct, or in the nucleic acid construct outside of the elements listed in step (b);

wherein, when the host cell is transformed with the nucleic acid construct(s), the polynucleotide library of interest is site-specifically integrated in the correct orientation in the chromosome of the host cell by the recombinase, whereby the first selectable marker is excised from the chromosome, and whereby the second selection marker is also integrated and expressed or any non-functional partial second selectable markers are recombined to form a functional second selection marker in the chromosome which is expressed.

In a third aspect, the invention relates to the resulting filamentous fungal host cells comprising in its chromosome in the following order:
i) a first recognition sequence of a site-specific recombinase or a region that is 5′ or 3′ of an integration site;
ii) a polynucleotide library of interest; and either
iii) a selection marker and a second recognition sequence of the recombinase; or
iv) a first partial selection marker, a second recognition sequence of the recombinase and a second partial selection marker,
   wherein the host cell expresses the polynucleotide library and the selection marker.

In a final aspect, the invention relates to a method of producing a polypeptide of interest, comprising the steps of:

a) cultivating a filamentous fungal host cell of the third aspect under conditions conducive to produce the polypeptide encoded by the polynucleotide library, and; optionally
b) recovering the polypeptide.

### BRIEF DESCRIPTION OF THE FIGURES

FIG. 1 shows a schematic drawing of vector p002.

FIG. 2 shows a schematic drawing of vector pFRT-GIAMG.

FIG. 3 shows a schematic drawing of vector p007.

FIG. 4 shows a schematic drawing of vector pFRT-BsAMG.

FIG. 5 shows a schematic drawing of vector pDAu571.

FIG. 6 shows a schematic drawing of vector pDAu703.

FIG. 7 shows schematic drawings of:
Top part: the amy2-region of the chromosome in A. oryzae host strain DAu716, where two FRT-sites have been inserted;
Middle part: the linearized vector pDAu724; and
Bottom part: the amy2-region of the chromosome in A. oryzae host strain DAu716 after FLP-mediated integration of pDAu724 by double-homologous recombination between the respective FRT-sites.

FIG. 8 shows a schematic drawing of vector pDLHD0075.

FIG. 9 shows a restriction map of plasmid pJfyS156.

FIG. 10 shows a restriction map of pQM43.

FIG. 11 shows a restriction map of pQM45.

### DEFINITIONS

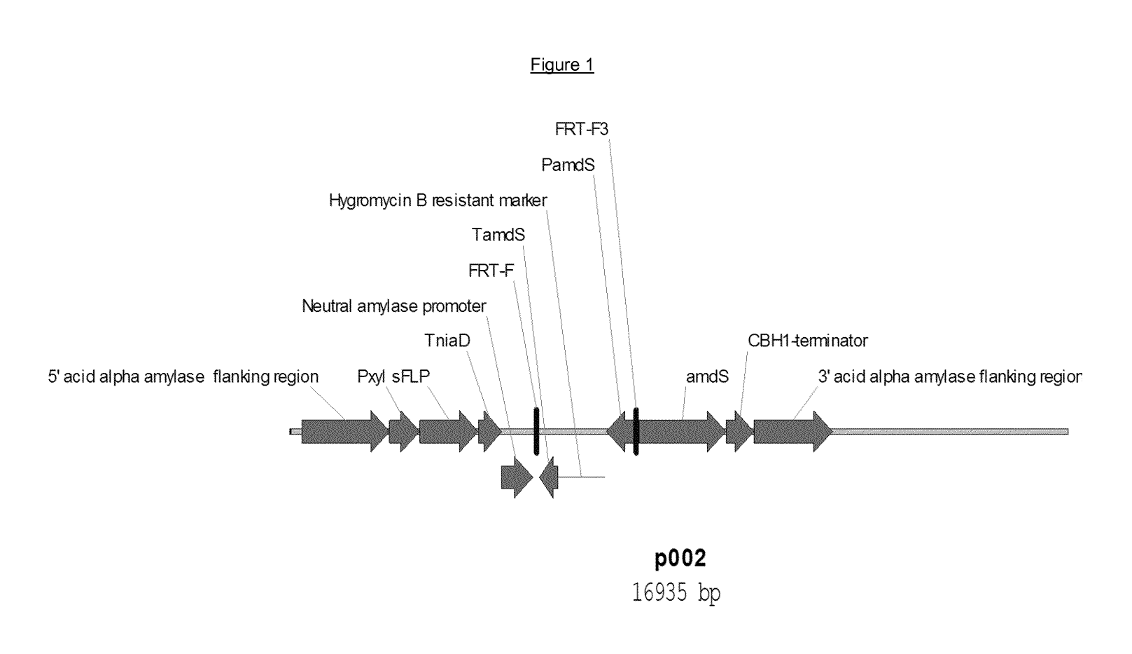

Cytosine deaminase: Cytosine deaminase (EC 3.5.4.1) catalyzes the deamination of cytosine and 5-fluorocytosine (5FC) to form uracil and toxic 5-fluorouracil (5FU), respectively. When genetically modified cells comprising cytosine deaminase are combined with 5FC it is converted to toxic 5FU, so the cytosine deaminase-encoding gene is potentially a potent negative selection marker. It has also been shown that an inhibitor in the pyrimidine de novo synthesis pathway can be utilized to create a condition in which cells are dependent on the conversion of pyrimidine supplements to uracil by cytosine deaminase. Thus, only cells expressing the cytosine deaminase gene can be rescued in a positive selection medium comprising an inhibitor of the pyrimidine de novo synthesis as well as inosine and cytosine (See FIG. 1 of Wei and Huber, 1996, J Biol Chem 271(7): 3812). The inhibitor is preferably N-(phosphonacetyl)-L-aspartate (PALA), which inhibits aspartate carbamyl transferase. If necessary, cytosine deaminase activity may be quantitated by a genetic assay (Frederico L. A. et al, 1990, Biochemistry 29: 2532-2537).

Allelic variant: The term “allelic variant” means any of two or more alternative forms of a gene occupying the same chromosomal locus. Allelic variation arises naturally through mutation, and may result in polymorphism within populations. Gene mutations can be silent (no change in the encoded polypeptide) or may encode polypeptides having altered amino acid sequences. An allelic variant of a polypeptide is a polypeptide encoded by an allelic variant of a gene.

Catalytic domain: The term “catalytic domain” means the region of an enzyme containing the catalytic machinery of the enzyme.

cDNA: The term “cDNA” means a DNA molecule that can be prepared by reverse transcription from a mature, spliced, mRNA molecule obtained from a eukaryotic or prokaryotic cell. cDNA lacks intron sequences that may be present in the corresponding genomic DNA. The initial, primary RNA transcript is a precursor to mRNA that is processed through a series of steps, including splicing, before appearing as mature spliced mRNA.

Coding sequence: The term “coding sequence” means a polynucleotide, which directly specifies the amino acid sequence of a polypeptide. The boundaries of the coding sequence are generally determined by an open reading frame, which begins with a start codon such as ATG, GTG, or TTG and ends with a stop codon such as TAA, TAG, or TGA. The coding sequence may be a genomic DNA, cDNA, synthetic DNA, or a combination thereof.

Control sequences: The term “control sequences” means nucleic acid sequences necessary for expression of a polynucleotide encoding a mature polypeptide of the present invention. Each control sequence may be native (i.e., from the same gene) or foreign (i.e., from a different gene) to the polynucleotide encoding the polypeptide or native or foreign to each other. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the polynucleotide encoding a polypeptide.

Expression: The term “expression” includes any step involved in the production of a polypeptide including, but not limited to, transcription, post-transcriptional modification, translation, post-translational modification, and secretion.

Expression vector: The term “expression vector” means a linear or circular DNA molecule that comprises a polynucleotide encoding a polypeptide and is operably linked to control sequences that provide for its expression.

Fragment: The term “fragment” means a polypeptide or a catalytic or binding domain having one or more (e.g., several) amino acids absent from the amino and/or carboxyl terminus of a mature polypeptide or domain; wherein the fragment has retained its catalytic or binding activity.

Host cell: The term “host cell” means any cell type that is susceptible to transformation, transfection, transduction, or the like with a nucleic acid construct or expression vector comprising a polynucleotide of the present invention. The term “host cell” encompasses any progeny of a parent cell that is not identical to the parent cell due to mutations that occur during replication.

Improved property: The term “improved property” means a characteristic associated with a variant that is improved compared to the parent. In the present invention, the improved property is increased expression yield of a variant relative to the parent.

Increased expression yield: The term “increased expression yield” means a higher amount (g) of secreted enzyme per liter of culture medium from cultivation of a host cell expressing the variant gene relative to the amount (g) of secreted active enzyme per liter produced under the same cultivation conditions by the same host cell expressing the parent gene. In one aspect, the variant has an increased expression yield compared to the parent enzyme of at least 1.05, at least 1.10, at least 1.20, at least 1.30, at least 1.40, at least 1.50, at least 1.60, at least 1.70, at least 1.80, at least 1.90, at least 2, at least 2.25, at least 2.50, at least 2.75, at least 3.00, at least 3.25, at least 3.50, at least 3.75, at least 4, at least 4.25, at least 4.50, at least 4.75, at least 5, at least 6, at least 7, at least 8, at least 9, or at least 10 fold.

Isolated: The term “isolated” means a substance in a form or environment that does not occur in nature. Non-limiting examples of isolated substances include (1) any non-naturally occurring substance, (2) any substance including, but not limited to, any enzyme, variant, nucleic acid, protein, peptide or cofactor, that is at least partially removed from one or more or all of the naturally occurring constituents with which it is associated in nature; (3) any substance modified by the hand of man relative to that substance found in nature; or (4) any substance modified by increasing the amount of the substance relative to other components with which it is naturally associated (e.g., recombinant production in a host cell; multiple copies of a gene encoding the substance; and use of a stronger promoter than the promoter naturally associated with the gene encoding the substance).

Mature polypeptide: The term “mature polypeptide” means a polypeptide in its final form following translation and any post-translational modifications, such as N-terminal processing, C-terminal truncation, glycosylation, phosphorylation, etc. It is known in the art that a host cell may produce a mixture of two of more different mature polypeptides (i.e., with a different C-terminal and/or N-terminal amino acid) expressed by the same polynucleotide. It is also known in the art that different host cells process polypeptides differently, and thus, one host cell expressing a polynucleotide may produce a different mature polypeptide (e.g., having a different C-terminal and/or N-terminal amino acid) as compared to another host cell expressing the same polynucleotide.

Mature polypeptide coding sequence: The term “mature polypeptide coding sequence” means a polynucleotide that encodes a mature polypeptide having catalytic or binding activity.

Nucleic acid construct: The term “nucleic acid construct” means a nucleic acid molecule, either single- or double-stranded, which is isolated from a naturally occurring gene or is modified to contain segments of nucleic acids in a manner that would not otherwise exist in nature or which is synthetic, which comprises one or more control sequences.

Operably linked: The term “operably linked” means a configuration in which a control sequence is placed at an appropriate position relative to the coding sequence of a polynucleotide such that the control sequence directs expression of the coding sequence.

Sequence identity: The relatedness between two amino acid sequences or between two nucleotide sequences is described by the parameter “sequence identity”.

For purposes of the present invention, the sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the—nobrief option) is used as the percent identity and is calculated as follows:

(Identical Residues×100)/(Length of Alignment−Total Number of Gaps in Alignment)

For purposes of the present invention, the sequence identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 5.0.0 or later. The parameters used are gap open penalty of 10, gap extension penalty of 0.5, and the EDNAFULL (EMBOSS version of NCBI NUC4.4) substitution matrix. The output of Needle labeled “longest identity” (obtained using the—nobrief option) is used as the percent identity and is calculated as follows:

(Identical Deoxyribonucleotides×100)/(Length of Alignment−Total Number of Gaps in Alignment)

Subsequence: The term “subsequence” means a polynucleotide having one or more (e.g., several) nucleotides absent from the 5′ and/or 3′ end of a mature polypeptide coding sequence; wherein the subsequence encodes a fragment having catalytic or binding activity.

Variant: The term “variant” means a polypeptide having catalytic or binding activity comprising an alteration, i.e., a substitution, insertion, and/or deletion, at one or more (e.g., several) positions. A substitution means replacement of the amino acid occupying a position with a different amino acid; a deletion means removal of the amino acid occupying a position; and an insertion means adding an amino acid adjacent to and immediately following the amino acid occupying a position. Examples of conservative substitutions are within the groups of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions that do not generally alter specific activity are known in the art and are described, for example, by H. Neurath and R. L. Hill, 1979, In, The Proteins, Academic Press, New York. Common substitutions are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, and Asp/Gly. Alternatively, the amino acid changes are of such a nature that the physico-chemical properties of the polypeptides are altered. For example, amino acid changes may improve the thermal stability of the polypeptide, alter the substrate specificity, change the pH optimum, and the like. Essential amino acids in a polypeptide can be identified according to procedures known in the art, such as site-directed mutagenesis or alanine-scanning mutagenesis (Cunningham and Wells, 1989, Science 244: 1081-1085). In the latter technique, single alanine mutations are introduced at every residue in the molecule, and the resultant mutant molecules are tested for activity to identify amino acid residues that are critical to the activity of the molecule. See also, Hilton et al., 1996, J. Biol. Chem. 271: 4699-4708. The active site of the enzyme or other biological interaction can also be determined by physical analysis of structure, as determined by such techniques as nuclear magnetic resonance, crystallography, electron diffraction, or photoaffinity labeling, in conjunction with mutation of putative contact site amino acids. See, for example, de Vos et al., 1992, Science 255: 306-312; Smith et al., 1992, J. Mol. Biol. 224: 899-904; Wlodaver et al., 1992, FEBS Lett. 309: 59-64. The identity of essential amino acids can also be inferred from an alignment with a related polypeptide. Single or multiple amino acid substitutions, deletions, and/or insertions can be made and tested using known methods of mutagenesis, recombination, and/or shuffling, followed by a relevant screening procedure, such as those disclosed by Reidhaar-Olson and Sauer, 1988, Science 241: 53-57; Bowie and Sauer, 1989, Proc. Natl. Acad. Sci. USA 86: 2152-2156; WO 95/17413; or WO 95/22625. Other methods that can be used include error-prone PCR, phage display (e.g., Lowman et al., 1991, Biochemistry 30: 10832-10837; U.S. Pat. No. 5,223,409; WO 92/06204), and region-directed mutagenesis (Derbyshire et al., 1986, Gene 46: 145; Ner et al., 1988, DNA 7: 127).

Mutagenesis/shuffling methods can be combined with high-throughput, automated screening methods to detect activity of cloned, mutagenized polypeptides expressed by host cells (Ness et al., 1999, Nature Biotechnology 17: 893-896). Mutagenized DNA molecules that encode active polypeptides can be recovered from the host cells and rapidly sequenced using standard methods in the art. These methods allow the rapid determination of the importance of individual amino acid residues in a polypeptide.

### DETAILED DESCRIPTION OF THE INVENTION

The present invention relates to methods for integrating a polynucleotide library of interest in the chromosome of a filamentous fungal host cell using a site-specific recombinase, polynucleotide library expression systems comprising a host cell and a polynucleotide construct, as well as the resulting filamentous fungal host cells comprising a polynucleotide library and their cultivation to produce a polypeptide.

In a preferred embodiment of the invention, the polynucleotide library is a gene library that comprises polynucleotides encoding variants of a polypeptide of interest; preferably the polypeptide of interest is an enzyme; more preferably the polypeptide of interest is a hydrolase, isomerase, ligase, lyase, oxidoreductase, or transferase, e.g., an aminopeptidase, amylase, carbohydrase, carboxypeptidase, catalase, cellobiohydrolase, cellulase, chitinase, cutinase, cyclodextrin glycosyltransferase, deoxyribonuclease, endoglucanase, esterase, alpha-galactosidase, beta-galactosidase, glucoamylase, alpha-glucosidase, beta-glucosidase, invertase, laccase, lipase, mannosidase, mutanase, oxidase, pectinolytic enzyme, peroxidase, phytase, polyphenoloxidase, proteolytic enzyme, ribonuclease, transglutaminase, xylanase, or beta-xylosidase.

In another preferred embodiment, the polynucleotide library comprises a library of control sequences, such as, a library of promoters, pro-regions, secretion signals, or terminators.

It is preferred that the filamentous fungal host cell of the invention is an Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma cell; preferably the host cell is an Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Thielavia terrestris, Trametes villosa, Trametes versicolor, Trichoderma harzianum, Trichoderma koningii, Trichoderma longibrachiatum, Trichoderma reesei, or Trichoderma viride cell.

In a preferred embodiment of the invention, the site specific recombinase is the Saccharomyces cerevisiae 2 μm flippase FLP, the phage TP901-1 integrase, the bacteriophage P1 CRE integrase, the bacterial XerC recombinase, the bacterial XerD recombinase, the lambda phage integrase or the HP1 integrase; preferably the site specific recombinase is the Saccharomyces cerevisiae 2 μm flippase FLP.

Two separate strategies may be applied to ensure that chromosomal integration in the chromosome of the host cell takes place in the correct orientation. If identical first and second recognition sequences are used, then the integration is likely to happen in both orientations, but the second selection marker may be tailored so that only correctly integrated fragments result in the expression of the second selection marker. On the other hand, it is also an option to use different first and second recognition sequences, for example, FRT-F and FRT-F3, in order to ensure that the fragment is integrated in a specific orientation. One aspect of the invention relates to the use of in vivo homologous recombination between a region of the nucleic acid construct that is transformed into the host cell with a region that is 5′ or 3′ of an intended integration site in the chromosome in combination with a recognition sequence of a site-specific recombinase in order to ensure that the polynucleotide library of interest is integrated in a specific orientation in the chromosome via the in vivo homologous recombination in combination with the site-specific recombination effected by the recombinase.

Accordingly, it is preferred that the first and second recognition sequences of the recombinase are identical or different; preferably the first and second recognition sequences of the recombinase are different in order to effect a directional integration of the polynucleotide library of interest in the host cell chromosome. Preferably, the first and second recognition sequences of the recombinase are different recognition sequences of the Saccharomyces cerevisiae 2 μm flippase FLP in order to effect a directional integration of the polynucleotide library of interest in the host cell chromosome; preferably the first and second recognition sequences of the recombinase are FRT-F (SEQ ID NO:27) and FRT-F3 (SEQ ID NO:28), respectively, or vice versa.

On the other hand, it is also preferably that one non-functional partial second selection marker is comprised in the host cell of the first aspect in step (a) and another non-functional partial second selection marker is comprised in the nucleic acid construct of the first aspect in step (b), wherein the partial second selection markers are recombined to form a functional second selection marker when the polynucleotide library of interest is site-specifically integrated in the correct orientation in the chromosome of the host cell by the recombinase via its recognition sequences; preferably one non-functional partial second selection marker comprises a promoter and, optionally, one or more intact 5′ exon of a polynucleotide encoding a selection marker, and, the other non-functional partial second selection marker comprises the remaining coding sequence of the selection marker.

In a preferred embodiment of the second aspect of the invention, one non-functional partial second selection marker is comprised in the host cell and another non-functional partial second selection marker is comprised in the nucleic acid construct, wherein the partial second selection markers are recombined to form a functional second selection marker when the polynucleotide library of interest is site-specifically integrated in the correct orientation in the chromosome of the host cell by the recombinase via its recognition sequences; preferably one non-functional partial second selection marker comprises a promoter and, optionally, one or more intact 5′ exon of a polynucleotide encoding a selection marker, and, wherein the other non-functional partial second selection marker comprises the remaining coding sequence of the selection marker.

In a preferred embodiment of the second aspect, the filamentous fungal host cell is transformed.

In a preferred embodiment of the third aspect, the first and second recognition sequences of the recombinase are identical or different; preferably the first and second recognition sequences of the recombinase are different.

In another preferred embodiment of the third aspect, the first and second recognition sequences of the recombinase are different recognition sequences of the Saccharomyces cerevisiae 2 μm flippase FLP; preferably the first and second recognition sequences of the recombinase are FRT-F (SEQ ID NO:27) and FRT-F3 (SEQ ID NO:28), respectively, or vice versa.

In another preferred embodiment, a region that is 5′ or 3′ of the integration site, i.e., that flanks 5′ or 3′ of the integration site, is used together with the second recognition sequence to effect directional integration of the polynucleotide library of interest at the integration site.

In a final preferred embodiment of the third aspect, the second recognition sequence of the recombinase is located in an intron separating the first partial selection marker from the second partial selection marker, as outlined in FIG. 7.

### Sources of Polypeptides Having Enzyme Activity

A polypeptide having enzyme activity of the present invention may be obtained from microorganisms of any genus. For purposes of the present invention, the term “obtained from” as used herein in connection with a given source shall mean that the polypeptide encoded by a polynucleotide is produced by the source or by a strain in which the polynucleotide from the source has been inserted. In one aspect, the polypeptide obtained from a given source is secreted extracellularly.

The polypeptide may be a bacterial polypeptide. For example, the polypeptide may be a Gram-positive bacterial polypeptide such as a Bacillus, Clostridium, Enterococcus, Geobacillus, Lactobacillus, Lactococcus, Oceanobacillus, Staphylococcus, Streptococcus, or Streptomyces polypeptide having [enzyme] activity, or a Gram-negative bacterial polypeptide such as a Campylobacter, E. coli, Flavobacterium, Fusobacterium, Helicobacter, Ilyobacter, Neisseria, Pseudomonas, Salmonella, or Ureaplasma polypeptide.

In one aspect, the polypeptide is a Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, or Bacillus thuringiensis polypeptide.

In another aspect, the polypeptide is a Streptococcus equisimilis, Streptococcus pyogenes, Streptococcus uberis, or Streptococcus equi subsp. Zooepidemicus polypeptide.

In another aspect, the polypeptide is a Streptomyces achromogenes, Streptomyces avermitilis, Streptomyces coelicolor, Streptomyces griseus, or Streptomyces lividans polypeptide.

The polypeptide may be a fungal polypeptide. For example, the polypeptide may be a yeast polypeptide such as a Candida, Kluyveromyces, Pichia, Saccharomyces, Schizosaccharomyces, or Yarrowia polypeptide; or a filamentous fungal polypeptide such as an Acremonium, Agaricus, Alternaria, Aspergillus, Aureobasidium, Botryosphaeria, Ceriporiopsis, Chaetomidium, Chrysosporium, Claviceps, Cochliobolus, Coprinopsis, Coptotermes, Corynascus, Cryphonectria, Cryptococcus, Diplodia, Exidia, Filibasidium, Fusarium, Gibberella, Holomastigotoides, Humicola, Irpex, Lentinula, Leptospaeria, Magnaporthe, Melanocarpus, Meripilus, Mucor, Myceliophthora, Neocallimastix, Neurospora, Paecilomyces, Penicillium, Phanerochaete, Piromyces, Poitrasia, Pseudoplectania, Pseudotrichonympha, Rhizomucor, Schizophyllum, Scytalidium, Talaromyces, Thermoascus, Thielavia, Tolypocladium, Trichoderma, Trichophaea, Verticillium, Volvariella, or Xylaria polypeptide.

In another aspect, the polypeptide is a Saccharomyces carlsbergensis, Saccharomyces cerevisiae, Saccharomyces diastaticus, Saccharomyces douglasii, Saccharomyces kluyveri, Saccharomyces norbensis, or Saccharomyces oviformis polypeptide.

In another aspect, the polypeptide is an Acremonium cellulolyticus, Aspergillus aculeatus, Aspergillus awamori, Aspergillus foetidus, Aspergillus fumigatus, Aspergillus japonicus, Aspergillus nidulans, Aspergillus niger, Aspergillus oryzae, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenaturn, Humicola grisea, Humicola insolens, Humicola lanuginosa, Irpex lacteus, Mucor miehei, Myceliophthora thermophila, Neurospora crassa, Penicillium funiculosum, Penicillium purpurogenum, Phanerochaete chrysosporium, Thielavia achromatica, Thielavia albomyces, Thielavia albopilosa, Thielavia australeinsis, Thielavia fimeti, Thielavia microspora, Thielavia ovispora, Thielavia peruviana, Thielavia setosa, Thielavia spededonium, Thielavia subthermophila, Thielavia terrestris, Trichoderma harzianurn, Trichoderma koningii, Trichoderma longibrachiatum, Trichoderma reesei, or Trichoderma viride polypeptide.

It will be understood that for the aforementioned species, the invention encompasses both the perfect and imperfect states, and other taxonomic equivalents, e.g., anamorphs, regardless of the species name by which they are known. Those skilled in the art will readily recognize the identity of appropriate equivalents.

Strains of these species are readily accessible to the public in a number of culture collections, such as the American Type Culture Collection (ATCC), Deutsche Sammlung von Mikroorganismen and Zellkulturen GmbH (DSMZ), Centraalbureau Voor Schimmelcultures (CBS), and Agricultural Research Service Patent Culture Collection, Northern Regional Research Center (NRRL).

The polypeptide may be identified and obtained from other sources including microorganisms isolated from nature (e.g., soil, composts, water, etc.) or DNA samples obtained directly from natural materials (e.g., soil, composts, water, etc.) using the above-mentioned probes. Techniques for isolating microorganisms and DNA directly from natural habitats are well known in the art. A polynucleotide encoding the polypeptide may then be obtained by similarly screening a genomic DNA or cDNA library of another microorganism or mixed DNA sample. Once a polynucleotide encoding a polypeptide has been detected with the probe(s), the polynucleotide can be isolated or cloned by utilizing techniques that are known to those of ordinary skill in the art (see, e.g., Sambrook et al., 1989, supra).

### Nucleic Acid Constructs

The present invention also relates to nucleic acid constructs comprising a polynucleotide of the present invention operably linked to one or more control sequences that direct the expression of the coding sequence in a suitable host cell under conditions compatible with the control sequences.

The polynucleotide may be manipulated in a variety of ways to provide for expression of the polypeptide. Manipulation of the polynucleotide prior to its insertion into a vector may be desirable or necessary depending on the expression vector. The techniques for modifying polynucleotides utilizing recombinant DNA methods are well known in the art.

The control sequence may be a promoter, a polynucleotide that is recognized by a host cell for expression of a polynucleotide encoding a polypeptide of the present invention. The promoter contains transcriptional control sequences that mediate the expression of the polypeptide. The promoter may be any polynucleotide that shows transcriptional activity in the host cell including mutant, truncated, and hybrid promoters, and may be obtained from genes encoding extracellular or intracellular polypeptides either homologous or heterologous to the host cell.

Examples of suitable promoters for directing transcription of the nucleic acid constructs of the present invention in a filamentous fungal host cell are promoters obtained from the genes for Aspergillus nidulans acetamidase, Aspergillus niger neutral alpha-amylase, Aspergillus niger acid stable alpha-amylase, Aspergillus niger or Aspergillus awamori glucoamylase (glaA), Aspergillus oryzae TAKA amylase, Aspergillus oryzae alkaline protease, Aspergillus oryzae triose phosphate isomerase, Fusarium oxysporum trypsin-like protease (WO 96/00787), Fusarium venenatum amyloglucosidase (WO 00/56900), Fusarium venenatum Dania (WO 00/56900), Fusarium venenatum Quinn (WO 00/56900), Rhizomucor miehei lipase, Rhizomucor miehei aspartic proteinase, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase V, Trichoderma reesei xylanase I, Trichoderma reesei xylanase II, Trichoderma reesei xylanase III, Trichoderma reesei beta-xylosidase, and Trichoderma reesei translation elongation factor, as well as the NA2-tpi promoter (a modified promoter from an Aspergillus neutral alpha-amylase gene in which the untranslated leader has been replaced by an untranslated leader from an Aspergillus triose phosphate isomerase gene; non-limiting examples include modified promoters from an Aspergillus niger neutral alpha-amylase gene in which the untranslated leader has been replaced by an untranslated leader from an Aspergillus nidulans or Aspergillus oryzae triose phosphate isomerase gene); and mutant, truncated, and hybrid promoters thereof. Other promoters are described in U.S. Pat. No. 6,011,147.

The control sequence may also be a transcription terminator, which is recognized by a host cell to terminate transcription. The terminator is operably linked to the 3′-terminus of the polynucleotide encoding the polypeptide. Any terminator that is functional in the host cell may be used in the present invention.

Preferred terminators for filamentous fungal host cells are obtained from the genes for Aspergillus nidulans acetamidase, Aspergillus nidulans anthranilate synthase, Aspergillus niger glucoamylase, Aspergillus niger alpha-glucosidase, Aspergillus oryzae TAKA amylase, Fusarium oxysporum trypsin-like protease, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase V, Trichoderma reesei xylanase I, Trichoderma reesei xylanase II, Trichoderma reesei xylanase III, Trichoderma reesei beta-xylosidase, and Trichoderma reesei translation elongation factor.

The control sequence may also be an mRNA stabilizer region downstream of a promoter and upstream of the coding sequence of a gene which increases expression of the gene.

The control sequence may also be a leader, a nontranslated region of an mRNA that is important for translation by the host cell. The leader is operably linked to the 5′-terminus of the polynucleotide encoding the polypeptide. Any leader that is functional in the host cell may be used.

Preferred leaders for filamentous fungal host cells are obtained from the genes for Aspergillus oryzae TAKA amylase and Aspergillus nidulans triose phosphate isomerase.

The control sequence may also be a polyadenylation sequence, a sequence operably linked to the 3′-terminus of the polynucleotide and, when transcribed, is recognized by the host cell as a signal to add polyadenosine residues to transcribed mRNA. Any polyadenylation sequence that is functional in the host cell may be used.

Preferred polyadenylation sequences for filamentous fungal host cells are obtained from the genes for Aspergillus nidulans anthranilate synthase, Aspergillus niger glucoamylase, Aspergillus niger alpha-glucosidase Aspergillus oryzae TAKA amylase, and Fusarium oxysporum trypsin-like protease.

The control sequence may also be a signal peptide coding region that encodes a signal peptide linked to the N-terminus of a polypeptide and directs the polypeptide into the cell's secretory pathway. The 5′-end of the coding sequence of the polynucleotide may inherently contain a signal peptide coding sequence naturally linked in translation reading frame with the segment of the coding sequence that encodes the polypeptide. Alternatively, the 5′-end of the coding sequence may contain a signal peptide coding sequence that is foreign to the coding sequence. A foreign signal peptide coding sequence may be required where the coding sequence does not naturally contain a signal peptide coding sequence. Alternatively, a foreign signal peptide coding sequence may simply replace the natural signal peptide coding sequence in order to enhance secretion of the polypeptide. However, any signal peptide coding sequence that directs the expressed polypeptide into the secretory pathway of a host cell may be used.

Effective signal peptide coding sequences for filamentous fungal host cells are the signal peptide coding sequences obtained from the genes for Aspergillus niger neutral amylase, Aspergillus niger glucoamylase, Aspergillus oryzae TAKA amylase, Humicola insolens cellulase, Humicola insolens endoglucanase V, Humicola lanuginosa lipase, and Rhizomucor miehei aspartic proteinase.

The control sequence may also be a propeptide coding sequence that encodes a propeptide positioned at the N-terminus of a polypeptide. The resultant polypeptide is known as a proenzyme or propolypeptide (or a zymogen in some cases). A propolypeptide is generally inactive and can be converted to an active polypeptide by catalytic or autocatalytic cleavage of the propeptide from the propolypeptide. The propeptide or prepropetide coding sequence may be obtained from the genes for Bacillus subtilis alkaline protease (aprE), Bacillus subtilis neutral protease (nprT), Myceliophthora thermophila laccase (WO 95/33836), Rhizomucor miehei aspartic proteinase, Humicola insolens cutinase, and Saccharomyces cerevisiae alpha-factor.

Where both signal peptide and propeptide sequences are present, the propeptide sequence is positioned next to the N-terminus of a polypeptide and the signal peptide sequence is positioned next to the N-terminus of the propeptide sequence.

It may also be desirable to add regulatory sequences that regulate expression of the polypeptide relative to the growth of the host cell. Examples of regulatory sequences are those that cause expression of the gene to be turned on or off in response to a chemical or physical stimulus, including the presence of a regulatory compound. In filamentous fungi, the Aspergillus niger glucoamylase promoter, Aspergillus oryzae TAKA alpha-amylase promoter, and Aspergillus oryzae glucoamylase promoter, Trichoderma reesei cellobiohydrolase I promoter, and Trichoderma reesei cellobiohydrolase II promoter may be used. Other examples of regulatory sequences are those that allow for gene amplification. In eukaryotic systems, these regulatory sequences include the dihydrofolate reductase gene that is amplified in the presence of methotrexate, and the metallothionein genes that are amplified with heavy metals. In these cases, the polynucleotide encoding the polypeptide would be operably linked to the regulatory sequence.

### Polynucleotide Constructs

The present invention also relates to recombinant polynucleotide constructs or expression vectors comprising a polynucleotide of the present invention, a promoter, and transcriptional and translational stop signals. The various nucleotide and control sequences may be joined together to produce a recombinant expression vector that may include one or more convenient restriction sites to allow for insertion or substitution of the polynucleotide encoding the polypeptide at such sites. Alternatively, the polynucleotide may be expressed by inserting the polynucleotide or a nucleic acid construct comprising the polynucleotide into an appropriate vector for expression. In creating the expression vector, the coding sequence is located in the vector so that the coding sequence is operably linked with the appropriate control sequences for expression.

The recombinant expression vector may be any vector (e.g., a plasmid or virus) that can be conveniently subjected to recombinant DNA procedures and can bring about expression of the polynucleotide. The choice of the vector will typically depend on the compatibility of the vector with the host cell into which the vector is to be introduced. The vector may be a linear or closed circular plasmid.

The vector may be an autonomously replicating vector, i.e., a vector that exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one that, when introduced into the host cell, is integrated into the genome and replicated together with the chromosome(s) into which it has been integrated. Furthermore, a single vector or plasmid or two or more vectors or plasmids that together contain the total DNA to be introduced into the genome of the host cell, or a transposon, may be used.

The vector preferably contains one or more selectable markers that permit easy selection of transformed, transfected, transduced, or the like cells. A selectable marker is a gene the product of which provides for biocide or viral resistance, resistance to heavy metals, prototrophy to auxotrophs, and the like.

Selectable markers for use in a filamentous fungal host cell include, but are not limited to, adeA (phosphoribosylaminoimidazole-succinocarboxamide synthase), adeB (phosphoribosyl-aminoimidazole synthase), amdS (acetamidase), argB (ornithine carbamoyltransferase), bar (phosphinothricin acetyltransferase), hph (hygromycin phosphotransferase), niaD (nitrate reductase), pyrG (orotidine-5′-phosphate decarboxylase), sC (sulfate adenyltransferase), and trpC (anthranilate synthase), as well as equivalents thereof. Preferred for use in an Aspergillus cell are Aspergillus nidulans or Aspergillus oryzae amdS and pyrG genes and a Streptomyces hygroscopicus bar gene. Preferred for use in a Trichoderma cell are adeA, adeB, amdS, hph, and pyrG genes.

The selectable marker may be a dual selectable marker system as described in WO 2010/039889. In one aspect, the dual selectable marker is an hph-tk dual selectable marker system.

The vector preferably contains an element(s) that permits integration of the vector into the host cell's genome or autonomous replication of the vector in the cell independent of the genome.

For integration into the host cell genome, the vector may rely on the polynucleotide's sequence encoding the polypeptide or any other element of the vector for integration into the genome by homologous or non-homologous recombination. Alternatively, the vector may contain additional polynucleotides for directing integration by homologous recombination into the genome of the host cell at a precise location(s) in the chromosome(s). To increase the likelihood of integration at a precise location, the integrational elements should contain a sufficient number of nucleic acids, such as 100 to 10,000 base pairs, 400 to 10,000 base pairs, and 800 to 10,000 base pairs, which have a high degree of sequence identity to the corresponding target sequence to enhance the probability of homologous recombination. The integrational elements may be any sequence that is homologous with the target sequence in the genome of the host cell. Furthermore, the integrational elements may be non-encoding or encoding polynucleotides. On the other hand, the vector may be integrated into the genome of the host cell by non-homologous recombination.

For autonomous replication, the vector may further comprise an origin of replication enabling the vector to replicate autonomously in the host cell in question. The origin of replication may be any plasmid replicator mediating autonomous replication that functions in a cell. The term “origin of replication” or “plasmid replicator” means a polynucleotide that enables a plasmid or vector to replicate in vivo.

Examples of origins of replication useful in a filamentous fungal cell are AMA1 and ANS1 (Gems et al., 1991, Gene 98: 61-67; Cullen et al., 1987, Nucleic Acids Res. 15: 9163-9175; WO 00/24883). Isolation of the AMA1 gene and construction of plasmids or vectors comprising the gene can be accomplished according to the methods disclosed in WO 00/24883.

More than one copy of a polynucleotide of the present invention may be inserted into a host cell to increase production of a polypeptide. An increase in the copy number of the polynucleotide can be obtained by integrating at least one additional copy of the sequence into the host cell genome or by including an amplifiable selectable marker gene with the polynucleotide where cells containing amplified copies of the selectable marker gene, and thereby additional copies of the polynucleotide, can be selected for by cultivating the cells in the presence of the appropriate selectable agent.

The procedures used to ligate the elements described above to construct the recombinant expression vectors of the present invention are well known to one skilled in the art (see, e.g., Sambrook et al., 1989, supra).

### Host Cells

The present invention also relates to recombinant host cells, comprising a polynucleotide of the present invention operably linked to one or more control sequences that direct the production of a polypeptide of the present invention. A construct or vector comprising a polynucleotide is introduced into a host cell so that the construct or vector is maintained as a chromosomal integrant.

The term “host cell” encompasses any progeny of a parent cell that is not identical to the parent cell due to mutations that occur during replication. The choice of a host cell will to a large extent depend upon the gene encoding the polypeptide and its source.

The host cell may be a fungal cell. “Fungi” as used herein includes the phyla Ascomycota, Basidiomycota, Chytridiomycota, and Zygomycota as well as the Oomycota and all mitosporic fungi (as defined by Hawksworth et al., In, Ainsworth and Bisby's Dictionary of The Fungi, 8th edition, 1995, CAB International, University Press, Cambridge, UK).

The fungal host cell may be a filamentous fungal cell. “Filamentous fungi” include all filamentous forms of the subdivision Eumycota and Oomycota (as defined by Hawksworth et al., 1995, supra). The filamentous fungi are generally characterized by a mycelial wall composed of chitin, cellulose, glucan, chitosan, mannan, and other complex polysaccharides. Vegetative growth is by hyphal elongation and carbon catabolism is obligately aerobic.

The filamentous fungal host cell may be an Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma cell.

For example, the filamentous fungal host cell may be an Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenaturn, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Thielavia terrestris, Trametes villosa, Trametes versicolor, Trichoderma harzianum, Trichoderma koningii, Trichoderma longibrachiatum, Trichoderma reesei, or Trichoderma viride cell.

Fungal cells may be transformed by a process involving protoplast formation, transformation of the protoplasts, and regeneration of the cell wall in a manner known per se. Suitable procedures for transformation of Aspergillus and Trichoderma host cells are described in EP 238023, Yelton et al., 1984, Proc. Natl. Acad. Sci. USA 81: 1470-1474, and Christensen et al., 1988, Bio/Technology 6: 1419-1422. Suitable methods for transforming Fusarium species are described by Malardier et al., 1989, Gene 78: 147-156, and WO 96/00787.

### Methods of Production

The present invention also relates to methods of producing a polypeptide of interest, comprising the steps of a) cultivating a filamentous fungal host cell of the third aspect under conditions conducive to produce the polypeptide encoded by the polynucleotide library, and; optionally, b) recovering the polypeptide.

The host cells are cultivated in a nutrient medium suitable for production of the polypeptide using methods known in the art. For example, the cells may be cultivated by shake flask cultivation, or small-scale or large-scale fermentation (including continuous, batch, fed-batch, or solid state fermentations) in laboratory or industrial fermentors in a suitable medium and under conditions allowing the polypeptide to be expressed and/or isolated. The cultivation takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection). If the polypeptide is secreted into the nutrient medium, the polypeptide can be recovered directly from the medium. If the polypeptide is not secreted, it can be recovered from cell lysates.

The polypeptide may be detected using methods known in the art that are specific for the polypeptides. These detection methods include, but are not limited to, use of specific antibodies, formation of an enzyme product, or disappearance of an enzyme substrate. For example, an enzyme assay may be used to determine the activity of the polypeptide.

The polypeptide may be recovered using methods known in the art. For example, the polypeptide may be recovered from the nutrient medium by conventional procedures including, but not limited to, collection, centrifugation, filtration, extraction, spray-drying, evaporation, or precipitation. In one aspect, a fermentation broth comprising the polypeptide is recovered.

The polypeptide may be purified by a variety of procedures known in the art including, but not limited to, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), SDS-PAGE, or extraction (see, e.g., Protein Purification, Janson and Ryden, editors, VCH Publishers, New York, 1989) to obtain substantially pure polypeptides.

In an alternative aspect, the polypeptide is not recovered, but rather a host cell of the present invention expressing the polypeptide is used as a source of the polypeptide.

The present invention is further described by the following examples that should not be construed as limiting the scope of the invention.

### EXAMPLES

Molecular cloning techniques are described in Sambrook, J., Fritsch, E. F., Maniatis, T. 30 (1989) Molecular cloning: a laboratory manual (2nd edn.) Cold Spring Harbor Laboratory, Cold Spring Harbor, N.Y.

Enzymes for DNA manipulations (e.g. restriction endonucleases, ligases etc.) were obtained from New England Biolabs, Inc. and were used according to the manufacturer's instructions.

### Media and Reagents

The following media and reagents were used unless otherwise specified; chemicals used for buffers and substrates were commercial products of analytical grade:
Cove: 342.3 g/L Sucrose, 20 ml/L COVE salt solution, 10 mM Acetamide, 30 g/L noble agar.
Cove top agar: 342.3 g/L Sucrose, 20 ml/L COVE salt solution, 10 mM Acetamide, 10 g/L low melt agarose.
Cove-N plates are composed of 30 g sucrose, 20 ml Cove salt solution, 3 g NaNO₃, and 30 g noble agar and water to 1 litre.
COVE salt solution is composed of 26 g KCl, 26 g MgSO₄ 7H₂0, 76 g KH₂PO₄ and 50 ml Cove trace metals and water to 1 litre.
Trace metal solution for COVE is composed of 0.04 g NaB₄0₇ 10H₂0, 0.4 g CuSO₄ 5H₂0, 1.2 g FeSO₄7H₂0, 1.0 g MnSO₄ H₂0, 0.8 g Neutral amylase 11 Mo0₂₂H₂0, and 10.0 g ZnSO₄ 7H₂0 and water to 1 litre.
¼ YPM is composed of 2.5 g yeast extract, 5 g pepton and 5 g maltose (pH 4.5) and water to 1 litre.
STC buffer is composed of 0.8 M sorbitol, 25 mM Tris (pH 8), and 25 mM CaCl2 and water to 1 litre.
STPC buffer is composed of 40% PEG4000 in STC buffer.
MLC is composed of 40 g Glucose, 50 g Soybean powder, 4 g/Citric acid (pH 5.0) and water to 1 litre.
Cellulase-inducing medium (CIM) was composed of 20 g of cellulose, 10 g of corn steep solids, 1.45 g of (NH₄)₂SO₄, 2.08 g of KH₂PO₄, 0.28 g of CaCl₂, 0.42 g of MgSO₄.7H₂O, 0.42 ml of Trichoderma trace metals solution, 1-2 drops of antifoam, and deionized water to 1 liter; pH adjusted to 6.0.
COVE plates were composed of 342.3 g of sucrose, 20 ml of COVE salt solution, 10 ml of 1 M acetamide, 10 ml of 1.5 M CsCl, 25 g of Noble agar (Difco), and deionized water to 1 liter.
COVE salt solution was composed of 26 g of KCl, 26 g of MgSO₄.7H₂O, 76 g of KH₂PO₄, 50 ml of COVE trace metals solution, and deionized water to 1 liter.
COVE trace metals solution was composed of 0.04 g of NaB₄O₇.10H₂O, 0.4 g of CuSO₄.5H₂O, 1.2 g of FeSO₄.7H₂O, 0.7 g of MnSO₄.H₂O, 0.8 g of Na₂MoO₂.2H₂O, 10 g of ZnSO₄.7H₂O, and deionized water to 1 liter.
LB+Amp medium was composed of 10 g of tryptone, 5 g of yeast extract, 5 g of NaCl, and deionized water to 1 liter. After autoclaving 1 ml of a 100 mg/ml solution of ampicillin in water was added.
PDA plates were composed of 39 g of Potato Dextrose Agar (Difco) and deionized water to 1 liter.
PEG buffer was composed of 500 g of polyethylene glycol 4000 (PEG 4000), 10 mM CaCl₂, 10 mM Tris-HCl pH 7.5, and deionized water to 1 liter; filter sterilized.
SOC medium was composed of 0.5 g of NaCl, 5 g of yeast extract, 20 g of tryptone, 10 ml of 250 mM KCl, and deionized water to 1 liter.
STC was composed of 1 M sorbitol, 10 mM CaCl₂, and 10 mM Tris-HCl, pH 7.5; filter sterilized.
TAE buffer was composed of 4.84 g of Tris Base, 1.14 ml of Glacial acetic acid, 2 ml of 0.5 M EDTA pH 8, and deionized water to 1 liter.
Trichoderma Minimal Medium (TrMM) plates (for sub-culturing) were composed of 30 g sucrose, 20 ml COVE salt solution, 0.6 g of CaCl₂.2H₂O, 6 g of (NH₄)₂SO₄, 25 g of Noble agar, and deionized water to 1 liter.
Trichoderma trace metals solution was composed of 216 g of FeCl₃.6H₂O, 58 g of ZnSO₄.7H₂O, 27 g of MnSO₄—H₂O, 10 g of CuSO₄.5H₂O, 2.4 g of H₃BO₃, 336 g of citric acid, and deionized water to 1 liter.
YP medium was composed of 10 g of yeast extract, 20 g of Bacto peptone, and deionized water to 1 liter.
2XYT plus ampicillin plates were composed of 16 g of tryptone, 10 g of yeast extract, 5 g of sodium chloride, 15 g of Bacto agar, and deionized water to 1 liter. One ml of a 100 mg/ml solution of ampicillin was added after the autoclaved medium was tempered to 55° C.

### Purchased Material and Strains

E. coli DH5-alpha (TOYOBO) was used for plasmid construction and amplification. Amplified plasmids were recovered with Qiagen Plasmid Kit (QIAGEN). Ligation was done with DNA ligation kit (TAKARA) or T4 DNA ligase (BOEHRINGER MANNHEIM). QIAQUICK™ Gel Extraction Kit (Qiagen) was used for the purification of PCR fragments and extraction of DNA fragment from agarose gel.

In-Fusion® cloning kit and the E. coli cells (Fusion-Blue) were used for constructing the pDAu571 expression vector as well as pDAu703. PCR amplifications are performed using Phusion® high fidelity DNA polymerase

The expression host strain Aspergillus niger NN059095 was isolated by Novozymes and is a derivative of Aspergillus niger NN049184 which was isolated from soil. NN059095 was genetically modified to disrupt expression of amyloglycosidase activities.

Trichoderma reesei strain 981-O-8 (D4) is a mutagenized strain of T. reesei RutC30 (ATCC 56765; Montenecourt and Eveleigh, 1979, Adv. Chem. Ser. 181: 289-301).

Trichoderma reesei strain AgJg115-104-7B1 (WO 2011/075677) is a ku70-derivative of T. reesei strain 981-O-8 (D4).

### Transformation of Aspergillus

Transformation of Aspergillus species can be achieved using the general well-known methods for yeast transformation. The Aspergillus niger host strain was inoculated into 100 ml YPG medium supplemented with 10 mM uridine and incubated for 16 hrs at 32° C. at 80 rpm. Pellets were collected and washed with 0.6 M KCl, and resuspended in 20 ml 0.6 M KCl containing a commercial glucanase product (GLUCANEX™, Novozymes NS, Bagsvaerd, Denmark) at a final concentration of 20 mg per ml. The suspension was incubated at 32° C. with shaking (80 rpm) until protoplasts were formed, and then washed twice with STC buffer. The protoplasts were counted with a hematometer and resuspended and adjusted in an 8:2:0.1 solution of STC:STPC:DMSO to a final concentration of 2.5×107 protoplasts/ml.

Approximately 4 pg of plasmid DNA was added to 100 pl of the protoplast suspension, mixed gently, and incubated on ice for 30 minutes. One ml of SPTC was added and the protoplast suspension was incubated for 20 minutes at 37° C. After the addition of 10 ml of 50 C Cove top agarose, the reaction was poured onto Cove agar plates and the plates were incubated at 32° C. for 5 days.

### PCR Amplification

5×PCR buffer (incl. MgCl₂) 20 μl
2.5 mM dNTP mix 10 μl
Forward primer (100 pM) 1 μl
Reverse primer (100 pM) 1 μl
Expand High Fidelity polymerase (Roche) 1 μl
Template DNA (50-100 ng/pl) 1 μl
Distilled water to 100 μl

### PCR Conditions:

1. 94° C. 2 min

2. 94° C. 0.5 min

3. 55° C. 0.5 min

4. 72° C. 1-2 min

2-4. 30 cycles

5. 72° C. 10 min

### MTP Cultivation for Enzyme Production:

Spores of Aspergillus libraries were inoculated in 0.5-1 ml of ¼YPM media in 96 deep well plate and cultivated at 30° C. for 2-3 days at 600 rpm.

### Southern Hybridization

Mycelia of selected transformants were harvested from overnight-culture in 100 ml YPG medium, rinsed with distilled water, dried and frozen at −80° C. Ground mycelia were incubated with Proteinase K and RNaseA at 65° C. for 1 hrs. Genome DNA was recovered by phenol/CHCl3 extraction twice followed by EtOH precipitation and resuspended in distilled water.

Non-radioactive probes were synthesized using a PCR DIG probe synthesis kit (Roche Applied Science, Indianapolis Ind.) followed by manufacture's instruction. DIG labeled probes were gel purified using a QIAQUICK™ Gel Extraction Kit (QIAGEN Inc., Valencia, Calif.) according to the manufacturer's instructions.

Five micrograms of genome DNA was digested with appropriate restriction enzymes completely for 16 hours (40 ul total volumes, 4 U enzyme/ul DNA) and run on a 0.8% agarose gel. The DNA was fragmented in the gel by treating with 0.2 M HCl, denatured (0.5 M NaOH, 1.5 M NaCl) and neutralized (1 M Tris, pH7.5; 1.5 M NaCl) for subsequent transfer in 20×SSC to HyBond N+ membrane (Amersham). The DNA was UV cross-linked to the membrane and prehybridized for 1 hour at 42° C. in 20 ml DIG Easy Hyb (Roche Diagnostics Corporation, Mannheim, Germany).

The denatured probe was added directly to the DIG Easy Hyb buffer and an overnight hybridization at 42° C. was done. Following the post hybridization washes (twice in 2×SSC, room temperature, 5 min and twice in 0.1×SSC, 68° C., 15 min. each), chemiluminescent detection using the DIG detection system and CPD-Star (Roche) was done according to the manufacturer's protocol. The DIG-labeled DNA Molecular Weight Marker II (Roche) was used for the standard marker.

### Example 1. An Aspergillus niger Host/Vector System for Inserting an Enzyme-Encoding Gene Library into the Genome Employing a Promoterless Selection Marker

An Aspergillus niger comprising an enzyme-encoding polynucleotide library inserted into its genome was constructed in two steps by first integrating a plasmid denoted p002 (full DNA sequence provided in SEQ ID NO:1 and schematic shown in FIG. 1), which comprised (in the following order):
a) a 5′ genomic flanking region of the Aspergillus niger acid alpha amylase-encoding gene;
b) an Aspergillus nidulans xylanase-gene promoter operably linked with a FLP recombinase-encoding gene and an Aspergillus oryzae nitrate reductase-gene terminator;
c) an Aspergillus neutral amylase-gene promoter,
d) a first recombinase recognition sequence:

(SEQ ID NO: 2)

5′ ttgaagttcctattccgagttcctattctctagaaagtataggaact
tc,

e) an amdS promoter operably linked with a hygromycin B resistance marker gene and an amdS terminator;
f) a second recombinase recognition sequence

(SEQ ID NO: 3)

5′ ttgaagttcctattccgagttcctattcttcaaatagtataggaact
tca,

g) an acetoamidase(amdS)-encoding gene without a promoter but with a terminator; and finally
h) a 3′ genomic flanking region of the Aspergillus niger acid alpha amylase-encoding gene.

The p002 plasmid was transformed into an A. niger strain 1007-8 (disclosed in WO121600093) followed by hygromycin B selection. The obtained transformants were confirmed by Southern blotting analysis to have the correctly integrated expected genomic sequence. A positive transformant was selected and protoplasts were made from the strain.

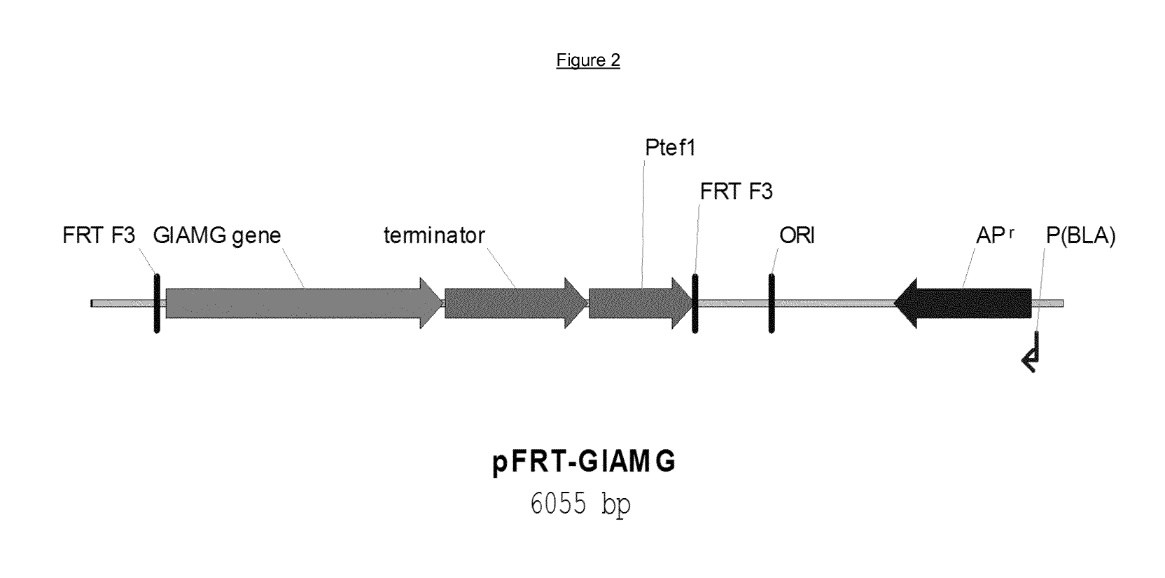

In a second step, an expression vector pFRT-GIAMG was constructed based on the pUC19 vector; the full DNA sequence of pFRT-GIAMG is provided in SEQ ID NO:4 and schematic shown in FIG. 2. The vector comprised (in the following order):
a) a recombinase recognition sequence,
b) a glucoamylase gene from Gloeophyllum trabeum (DNA in SEQ ID NO:5; amino acid sequence in SEQ ID NO:6) operably linked with an A. niger glucoamylase terminator;
c) an A. oryzae translation elongation factor 1 (TEF1) promoter to drive the expression of a genomic promoterless selection marker in the genome of the host cell after integration, thereby also ensuring properly oriented integration; and
d) a recombinase recognition sequence.

One μg of plasmid was transformed into the protoplasts to generate 200-300 colonies on Cove minimal plates (Cove D. J. 1966. Biochem. Biophys. cta. 113:51-56) supplemented with 1.0 M sucrose as carbon source and 1% xylose. Colonies were inoculated to czapec-dox plates containing 0.1% amylopectin to confirm glucoamylase activity by iodine-staining (0.15% I₂/1.5% KI). One positive transformant was selected and denoted A. niger 1007-002-44-11.

### Example 2. An A. niger Host/Vector System Employing a Promoterless Partial Selection Marker

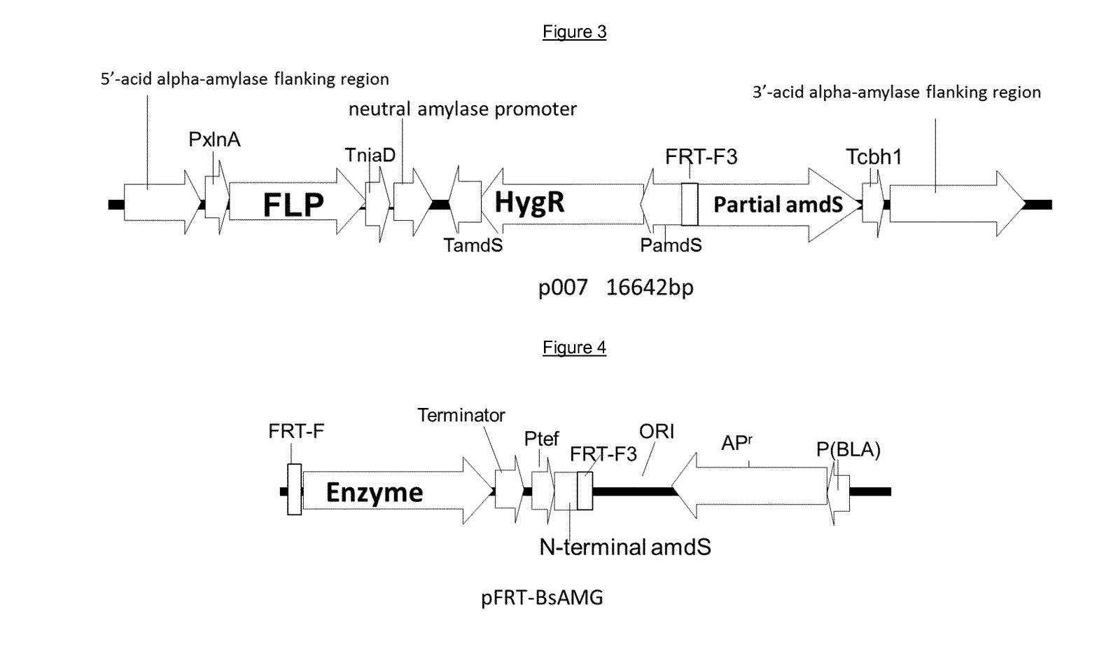

A plasmid, p007 (FIG. 3; SEQ ID NO:7), was constructed as follows: A first PCR fragment was generated with a primer pair of SpeI-FRTF3-amdS and amdS-F-probe with plasmid p002 as a template to amplify the DNA region of SpeI site-a recognition sequence-C-terminal of amdS (4.0 kbp). The amplified PCR product was then digested by SpeI and PacI.

Primer SpeI-FRTF3-amdS
(81mer; SEQ ID NO: 8):
5′ ggcgtagactagttgaagttcctattccgagtcctattcttcaaata
gtataggaacttcatcagggagatgtaacaac
Primer amdS-F-probe
(22mer; SEQ ID NO: 9):
5′ ctatggagtcaccacatttccc

A DNA fragment (1.0 kbp) comprising a recombinase recognition sequence and a neutral amylase promoter operably linked with an enzyme-encoding gene, was prepared by double digestion of plasmid p002 by PacI and BglII.

A DNA fragment (3.0 kbp) which contains the amdS promoter operably linked to the hygromycin resistance-encoding gene and the amdS terminator was prepared by double digestion of p002 by BglII and SpeI.

The three DNA fragments were ligated together and transformed into E. coli DH5alpha. The resultant plasmid was named p007.

The p007 plasmid comprised a promoter operably linked with a FLP recombinase gene and a terminator, an additional NA2 promoter, a hygromycin gene with a promoter and terminator between FRTs, and a partial amdS selection marker gene lacking the N-terminal sequence and half of its first intron, all flanked by two acid alpha-amylase-flanking genomic regions.

Plasmid p007 was transformed into A. niger strain 1007-8 (disclosed in WO121600093) followed by hygromycin B selection. The obtained transformants were confirmed by Southern blotting analysis to have the correct integrated sequences.

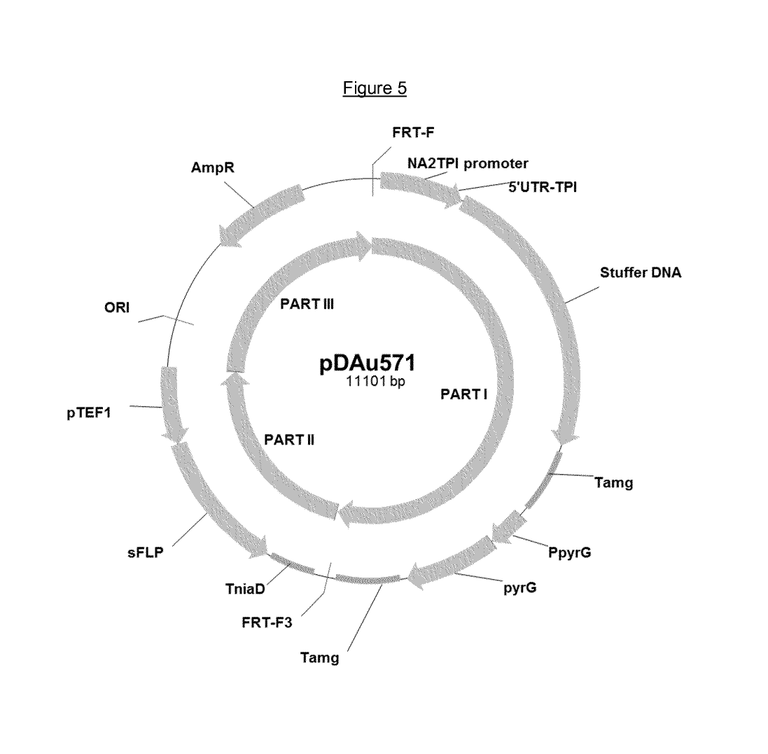

An expression vector, pFRT-BsAMG (FIG. 4; SEQ ID NO:10), was constructed based on the pUC19 vector which comprised (in the following order):
a) a recombinase recognition sequence,
b) the glucoamylase gene from Byssocorticium (DNA sequence shown in SEQ ID NO:11, the encoded amino acid sequence in SEQ ID NO:12) or a mutated glucoamylase-encoding gene as well as the A. niger glucoamylase terminator,
c) the A. oryzae TEF1 promoter (intended to drive expression of a selection marker in the genome of a recipient host cell) operably linked with a partial N-terminal acetamidase, amdS, gene; and
d) a recombinase recognition sequence.

The vector was constructed by inserting the partial acetamidase gene shown in SEQ ID NO:13 between the TEF1 promoter (Ptef1) and the second recognition sequence, and exchanging the glucoamylase gene from Gloeophyllum to the Byssocorticium glucoamylase gene.

A PCR was carried out using M13M4 and M13RV primers on pFRT-BsAMG as a template and a 4 kb fragment containing the first recombinase recognition sequence, the glucoamylase gene from Byssocorticium (or the mutated glucoamylase gene), the A. niger glucoamylase terminator, the TEF1 promoter, the partial N-terminal acetoamidase gene and the second recombinase recognition sequence were amplified.

Primer M13 RV
(SEQ ID NO: 14):
caggaaacagctatgac
Primer M13 M4
(SEQ ID NO: 15):
gttttcccagtcacgac

Both pFRT-BsAMG and the PCR fragment was transformed and cultivated on Cove minimal plates and 1% xylose. Colonies were inoculated to czapec-dox plates containing 0.1% amylopectin to confirm glucoamylase activities by iodine-staining (0.15% I₂/1.5% KI).

### Example 3. Single-Copy Site-Directed Gene-Insertion in A. niger

Plasmid Library Construction Using in-Fusion Cloning (Clontech)

40 ng of the pFRT-GIAMG expression vector was digested with restriction enzymes XhoI and BsiW1 to cut out the residing AMG-encoding gene. Two PCRs were carried out for with 2 primer pairs, a forward degenerate primer and a primer having more than 15 bp overlapping with an expression vector, and M13 M4 and a reverse primer having 15 bp overlapping with the degenerate primer using the pFRT-GIAMG vector as a template:

Primer In fusion vector R
(SEQ ID NO: 16):
tatgcgttatcgtacgcac
Primer M13 M4
(SEQ ID NO: 17):
gttttcccagtcacgac

The primer pair for a BsAMG library is shown below:

Primer M49X F
(27mer; SEQ ID NO: 18):
gtcaacccggactacnnktacacatgg
Primer M49X R
(18mer; SEQ ID NO: 19):
gtagtccgggttgacttg

The digested vector and PCR fragments were mixed with In-Fusion mix and transformed into E. coli DH5alpha. Obtained E. coli transformants were pooled and plasmids were extracted for library construction.

### PCR Library Construction

The first PCRs were carried out with two primer pairs, a degenerate primer and SOE-MR R and SOE-M4 F and a counterpart reverse primer of the degenerate primer using an pFRT expression vector as a template.

The second SOE PCR was carried out with a primer pair, SOE-F and SOE-R, and the PCR fragments from the first PCR. The resultant 4 kb fragments were recovered as PCR fragment libraries for Aspergillus library construction.

Primer SOE-M4 F
(SEQ ID NO: 20):
5′ gtactatctggcattggtacgttttcccagtcacgac
Primer SOE-MR R
(SEQ ID NO: 21):
5′ tggttatgatttcggcgatgcaggaaacagctatgac
Primer SOE-F
(SEQ ID NO: 22):
5′ gtactatctggcattggtac
Primer SOE-R
(SEQ ID NO: 23):
5′ tggttatgatttcggcgatg

One μg of each plasmid or PCR fragment library was transformed into the A. niger 1007-002-44-11 host strain. Transformants were isolated in a 96 well-MTP containing COVE-N gly agar (100 μl/well) and cultivated at 32° C. for 1 week to have enough sporulation. 100 μl/well of 0.01% Tween 20 was added to each well and the spore suspension was inoculated in a 96 well-MTP containing YPG and cultivated for 3 days at 30° C. with shaking. The libraries were then screened.

DNA Isolation from Aspergillus Clones

Inserted DNA of Aspergillus strains was amplified by the direct colony PCR method described below or by PCR on isolated chromosomal DNA using the primer pair “insert rescue F” and “R”.

Primer insert rescue F
(SEQ ID NO: 24):
5′ aatctcagaacaccaatatc
Primer insert rescue R
(SEQ ID NO: 25):
5′ aacactatgcgttatcgtac

Colony PCR was carried out as follows: Conidias were added to a 1.5 ml tube, 500 μl of TE-buffer was added and mixed briefly. The tube-content was diluted 10-20 times in water and one μl of the diluted mixture was used as template for PCR. The amplified DNA was purified by agarose gel electrophoresis and the QIAquick Gel Extraction kit (Qiagen) and then sequenced to check the quality of constructed libraries. The libraries were correct.

### Example 4. An Aspergillus oryzae Host/Vector System

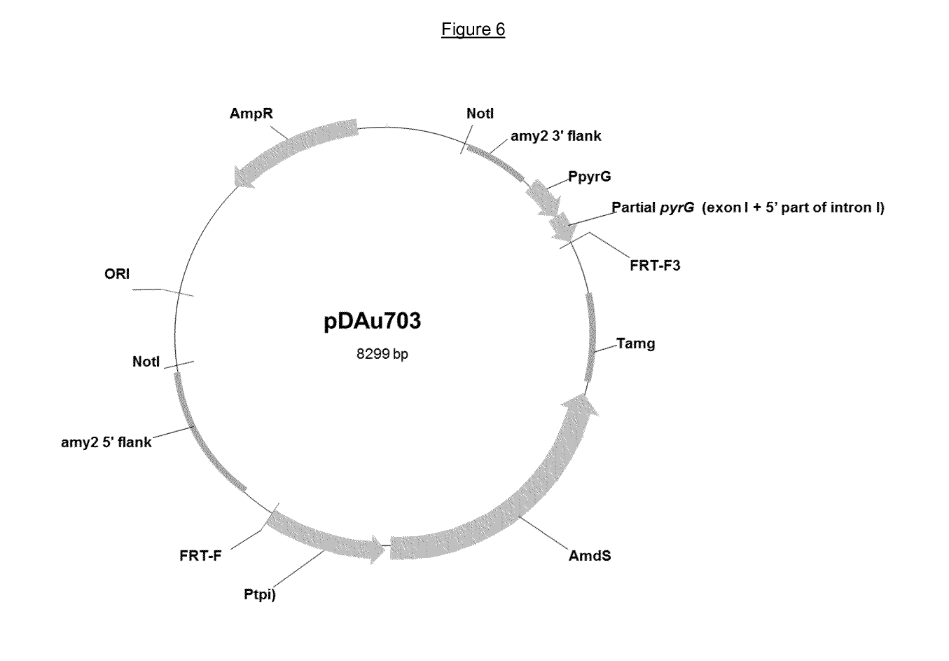

A. oryzae strain JaL1394 (disclosed in WO 2012/160093) was used as host strain in this example. We have constructed an integration expression vector denoted pDAu571 (FIG. 5; SEQ ID NO: 26) for site-specific recombination using the FLP/FRT system into the chromosome of A. oryzae JaL1394.

The vector pDAu571 is composed of three parts (PART-I, PART-II, PART-III; FIG. 5):

PART-I is delimited by two short FRT (Flippase Recognition Target) sites. In between the FRT sites the following elements are found:
An expression cassette with a “stuffer sequence” that can be replaced with any gene or polynucleotide library of interest (GOI) using the unique restriction sites BamHI and XhoI.
An Aspergillus nidulans PyrG selection marker to select the transformed host cells for their ability to grow on minimal medium without supplementation of uridine; the PyrG marker is operably linked with its native promoter and the terminator of the A. niger glucoamylase gene (Tamg) (pos. 4057-5918 of SEQ ID NO:26).

This part of the plasmid is to be integrated into the chromosome of the host organism via FLP-assisted recombinations between the respective FRT sites in the host and the vector. When evaluating gene libraries it is advantageous to ensure that PART-I of the vector is integrated in the same orientation in all transformants. This reduces any variation in gene expression from the flanking regions, e.g. from read-through. There is a small difference between the sequences of the FRT-F and FRT-F3 sites in the chromosome and the vector which ensures the correct orientation of the integrated part in the chromosome. The FRT-F3 sites in the chromosome of the host and in the vector will be recombined with each other by the FLP flippase and so will the FRT-F sites.

FRT-F (pos. 1-49 of SEQ ID NO: 26):

(SEQ ID NO: 27)

5′-ttgaagttcctattccgagttcctattctctagaaagtataggaact
tc
FRT-F3 (pos. 5925-5974 of SEQ ID NO: 26):

(SEQ ID NO: 28)

5′-ttgaagttcctattccgagttcctattcttcaaatagtataggaact
tca

The expression cassette comprises an artificial NA2TPI promoter constructed from the A. niger neutral amylase II promoter fused to the A. nidulans triose phosphate isomerase non-translated leader sequence (pos. 73-814 of SEQ ID NO:26).

In addition, the expression cassette comprises the so-called “stuffer sequence” which in this case is a gene that encodes lipoxygenase I from soybean (pos. 824-3343 of SEQ ID NO:22). As already mentioned, the stuffer sequence in the vector may be replaced with any other gene of interest using the two unique restriction enzyme sites BamHI and XhoI.

Finally, the expression cassette comprises the terminator of the A. niger glucoamylase gene (pos. 3358-4048 of SEQ ID NO:26).

PART-II of the pDau571 vector is a synthetic version of the flippase-encoding gene (sFLP) (pos. 4875-6143 of SEQ ID NO:26 (reverse strand)). The flippase gene expression is controlled by the A. oryzae translation-elongation factor 1 alpha promoter (pTEF1) (pos. 7759-8447 of SEQ ID NO:26 (reverse strand)) as well as the A. oryzae nitrate reductase terminator (TniaD) (pos. 5991-6478 of SEQ ID NO:26 (reverse strand)).

PART-III of the pDau571 vector is necessary for the propagation of the plasmid in E. coli and comprises an E. coli beta-lactamase (ampR) selectable marker and an E. coli origin of replication derived from the well-known pUC plasmid (pos. 8448-11101 of SEQ ID NO:26).

Plasmid pDAu571, wherein the stuffer sequence is replaced by a polynucleotide library of interest is transformed into the protoplasts of the strain Jal1394 as follows:

100 μl protoplasts are incubated with 10 μl miniprep plasmid DNA (undigested or linearized) or a PCR product spanning PART-I and -II of pDAu571 and 300 μl 60% PEG for 20 min at room temperature; then 5 ml Topagar (+10 mM NaNO₃) is added and the whole transformation mixture is plated on Sucrose/10 mM NaNO₃ plates. The plates are then incubated at 37° C. until spores can be spotted. To purify single transformants, colonies are restriked onto a new sucrose/10 mM NaNO₃ plate and incubated at 37° C. until spores develop.

The transformants obtained can be verified for the correct integration at the amy2 locus of the expression cassette by PCR using diagnostic primers located within the amy2 locus on both sides flanking the expression cassette including FRT sites and gene specific primers located within the expression cassette.

### Example 5. Construction of a Split-Marker Aspergillus oryzae Host/Vector System

The previous example provided an expression vector for flippase-mediated site-specific recombination and integration into a suitable filamentous fungal host cell. The correct orientation in the chromosome of the integrated expression cassette was ensured by using different recombinase recognition sequences.

Another way to ensure the proper orientation is to employ a split selection marker, where one non-functional part of the marker resides in the host chromosome and another non-functional part of the marker is on the incoming vector. Only the correctly oriented integration then results in a functional second selection marker. That split-marker principle is illustrated in this example; here the second selection marker is oriented in one direction but it could just as well have been oriented the other way.

### Media and Solutions Necessary for Aspergillus Protoplast Transformation and Selection of Recombinant Cells:

Trace metal

| | | |
|---|---|---|
| Na₂B₄O₇.10aq | 40 | mg/l |
| CuSO₄.5aq | 400 | mg/l |
| FeSO₄.7aq | 1200 | mg/l |
| MnSO₄.aq | 700 | mg/l |
| Na₂MoO₂.2aq | 800 | mg/l |
| ZnSO₄.7aq | 10.000 | mg/l |

Salt solution

| | | |
|---|---|---|
| KCl | 26 | g/l |
| MgSO₄.7aq | 26 | g/l |
| KH₂PO₄ | 76 | g/l |
| Trace metal | 50 | ml/l |

COVE medium
20 ml salt solution
20 g agar
218 g sorbitol
H₂O ad 1 l
Autoclave and then add:
50 ml 20% glucose
10 ml 1M urea.

Cove-N-gly slant

| | | |
|---|---|---|
| Salt solution | 50 | ml |
| Sorbitol | 218 | g |
| kaliumnitrat | 2.02 | g |
| Glycerol | 10 | ml |
| Agar | 35 | g |
| MilliQ H₂O to | 1000 | ml |

Sucrose medium
20 ml salt solution.
342 g sucrose.
H₂O ad 1 l
Autoclave and then add:
10 mM NaNO3
ST
0.6 M sorbitol
100 mM Tris/HCl pH 7.0
STC
1.2 M sorbitol
10 mM CaCl₂
10 mM Tris/HCl pH 7.5.
PEG
60% (W/V) PEG 4000 (BDH) (6 g PEG + ~5 ml
sterile water, put at 60-65° C.)
10 mM CaCl₂ (50 μl of a 2M CaCl₂)
10 mM Tris/HCl pH 7.5. (100 μl of a 1M Tris)
Sucrose agar plate
10 g Agar
10 ml Salt solution
1M sucrose to 500 ml
Autoclave to sterilized
Acetamide plates
10 ml salt solution
10 g agar
1M sucrose ad 500 ml¹.
autoclave.
Cool to approx. 65° C. and add 10 mM
acetamide and 15 mM CsCl.
Triton X-100 50 μl for 500 ml (only in the
restriking plates)

Introduction of the FRT Sites at the Amy2 Locus in Aspergillus oryzae DAu716

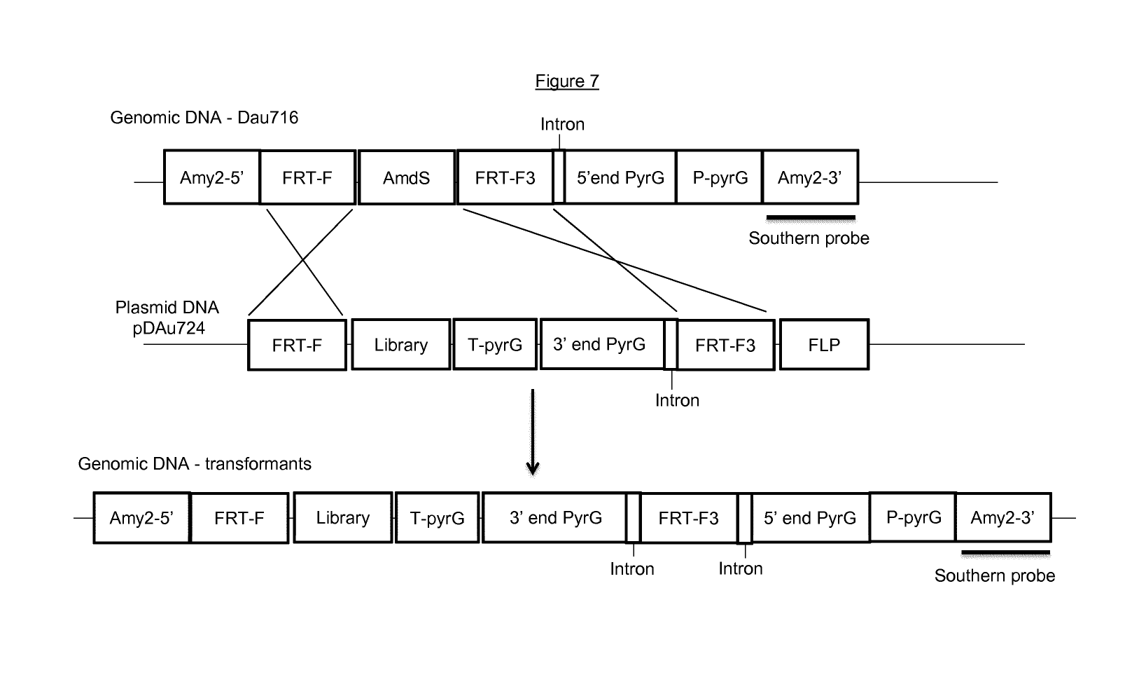

The plasmid pJAI1258 (described in WO12160097A1) was modified resulting in a plasmid denoted pDAu703. Plasmid pDAu703 contains the following elements in order (FIG. 6; SEQ ID NO:29):
amy2-3′ flank (490 bp); positions 449-938 of SEQ ID NO:29;
pyrG promoter operably linked with a partial pyrG gene containing the 5′-end of the pyrG CDS (the first exon and 5′ end of its first intron);
a FRT-F3 site (50 bp); positions 1452-1501 of SEQ ID NO:29;
an A. niger AMG terminator (Tamg) operably linked with the AmdS-encoding gene, positions 1511-2200 of SEQ ID NO:29;
A. nidulans acetamidase gene (AmdS), positions 2232-4131 of SEQ ID NO:29;
the strong triose-phosphate isomerase promoter (Ptpi) operably linked with the Amds-encoding gene; this allows growth on acetamide and CICs even though only one copy of the AmdS selection cassette is present in the genome as expected if the plasmid pDAU703 is integrated in one copy at the amy2 locus at FRT sites. Positions 4140-4894 of SEQ ID NO:29;
a FRT-F site (49 bp); positions 4903-4951 of SEQ ID NO:29;
amy2-5′ flank (1114 bp); positions 4964-6077 of SEQ ID NO:29;
The rest of the plasmid is composed of a part of DNA necessary for the maintenance of the plasmid as a replicative plasmid in the bacterial host cell E. coli (E. coli origin of replication and ampicillin resistance cassette).

Plasmid DNA pDAu703 was digested with NotI restriction enzyme to separate the DNA containing the integration cassette from the now irrelevant E. coli part of the plasmid.

The linearized plasmid pDAu703 was transformed into protoplasts of A. oryzae strain Jal1338 (disclosed in WO12160097A1) using a standard procedure described, for example, in WO98/01470 but with supplementing the media with 10 mM uridine since the strain is PyrG minus and therefore cannot grow in absence of uridine. Transformants were selected on AmdS selection plates

The resulting recombinant host strains have had the two FRT sites as well as the 5′ end of the split PyrG marker (first exon and part of the native intron) operably linked with its own promoter integrated by homologous recombination at the amy2 locus, as shown in the top panel of FIG. 7. The correct integration at the amy2 locus was checked by Southern blot analysis using a probe that annealed to the amy2 3′ end (FIG. 7). Integration of the FRT cassette generated hybridization signals at 5114 bp and 2637 bp in EcoRI and XhoI digests, respectively (not shown). This pattern is different from the Jal1338 host, where the amy2 locus is not disrupted. A correct strain was selected and denoted A. oryzae DAu716 (FIG. 7, top).

Transformation of DAu716 with the pDAU724 Vector Carrying a Lipase-Encoding Gene

This example demonstrates how the FRT/FLP recombination and split PyrG marker can be used to effectively make single copy insertions of an expression cassette with a high frequency in A. oryzae. We used the lipase gene from Thermomyces lanuginosa (e.g. disclosed in WO2008008950) as a reporter to measure the level of lipase produced in a transformed host.

Like in the previous example, an expression vector was constructed so that part of it can be integrated into the chromosome of the host cells at the FRT-sites using flippase as site specific recombination mediator. The part of the plasmid that is to be integrated in the genome carries a lipase gene operably linked with the NA2/TPi promoter and the terminator of the A. niger AMG gene. In order to be able to select the recombinant cells that have successfully integrated the expression cassette via the FRT sites, the remainder of the pyrG selection marker is also included in between the FRT sites. The promoter and the first exon resides in the DAu716 host and the remainder of the pyrG marker resides on the incoming plasmid. Upon site specific recombination, the PyrG marker will be reconstructed as an intact gene (with a FRT sequence inside its first intron which will, of course, be spliced out from the mRNA) and the recombinant cells will be able to express PyrG and grow on plate with NaNO₃ as sole nitrogen source.

Plasmid pDAU724 (FIG. 7, middle; SEQ ID NO:30) consists of:

PART-I which is to be integrated in the genomic DNA of the Aspergillus host cells and it consists of the two FRT sites with the expression cassette and one part of the split pyrG marker;

PART-II which will not be integrated in the genome of the host cell and which contains the FLPase expression cassette as well as E. coli selection marker and origin of replication.

The strain DAu716 was grown on a slant of Cove-N-gly medium until spores could be seen.

10-20 ml of Sucrose medium or YPD medium was added to the slant, and the spores were suspended by vortexing the slant. The spore suspension was transferred to a polycarbonate shakeflask (500 ml) containing 100 ml sucrose medium with 10 mM NaNO₃ (or other nitrogen source). The flask was incubated at 30° C. for 24 hr (200 rpm).

The mycelium was collected by filtration through miracloth and washed using 200 ml 0.6 M MgSO₄. The remaining liquid was squeezed out of the mycelium e.g. using a plastic pipette.

1-2 g of the mycelium was transferred to a small (100 ml) polycarbonate flask containing:

75-150 mg Glucanex

10 ml 1.2 M MgSO₄

100 ul 1 M NaH₂PO₄ pH 5.8

and the mycelium was suspended, 1 ml of 12 mg/ml BSA (sterile filtered) was added The suspension was incubated at 37° C. for ½-2 hr, and the protoplasting was monitored frequently by microscopy.

The protoplast suspension was filtered through miracloth into a 25 ml centrifuge tube and the suspension was overlaid with 5 ml ST (being careful not to mix up the lower layer). The resulting protoplasts were banded by centrifugation (2500 rpm/1350 g, 15 min, slow acceleration). The interface band of protoplasts was recovered and transferred to a fresh tube.

The protoplasts were diluted with 2 volumes of STC followed by centrifugation (2500 rpm/1350 g, 5 min). The protoplasts were then washed twice with 5 ml STC (using resuspension and centrifugation), and then resuspended in STC to a concentration of approx 5×10⁷ protoplasts/ml.

For each transformation, the transforming DNA was added at the bottom of e.g. a 14 ml tube, and 100 μl of protoplasts were added. 300 μl of PEG was added, and the tube was gently mixed by hand. After 20 minutes of incubation (RT), 6 ml top agar at temperature of 50° C. was added and immediately the suspension was poured on to a selective sucrose agar plate with 10 mM Na NO₃.

The plates were incubated at 37° C. until transformants were clearly visible and started to sporulate. 20 transformants were restriked onto a new selection plate with triton to isolate colonies that could be further analyzed by fermentation, Southern blot analysis or enzyme activity assay.

It was verified that the residing AmdS marker in the chromosome had been replaced by the incoming lipase gene in the transformants by streaking the transformants on plates containing CsCl (an inhibitor of the endogenous acetamidase) and acetamide as sole nitrogen source. Correct transformants should not be able to grow on these plates. We tested 20 recombinant cells obtained after transformation of pDAu724 into DAu716 and only a slight growth phenotype was observed compared to the parent host strain DAu716, where the AmdS selection marker is still present.

It was confirmed that all 20 transformants contained one inserted copy of the lipase expression cassette correctly inserted at the FRT sites.

The 20 transformants were inoculated in 3 ml YPD in a Uniplate® 10 ml 24 deep-wells plate (Whatman) sealed with Airpore tape (Quiagen) and incubated at 30 degree Celcius for 4 days with 200 rpm agitation. The supernatants were collected for further analysis (lipase assay and SDS-page) and the mycelia were also collected for genomic extraction and Southern analysis.

The 20 transformants showed comparable lipase activities in a lipase assay as well as comparable lipase protein levels on an SDS-PAGE gel. In addition, a Southern blot confirmed that all 20 transformants had only the expected single lipase gene copy correctly integrated in the chromosome.

### Example 6: Cloning of the Wild-Type Vigna angularis Xyloglucan Endotransglycosylase 16 (VaXET16) for Expression in an Aspergillus oryzae Screening Strain

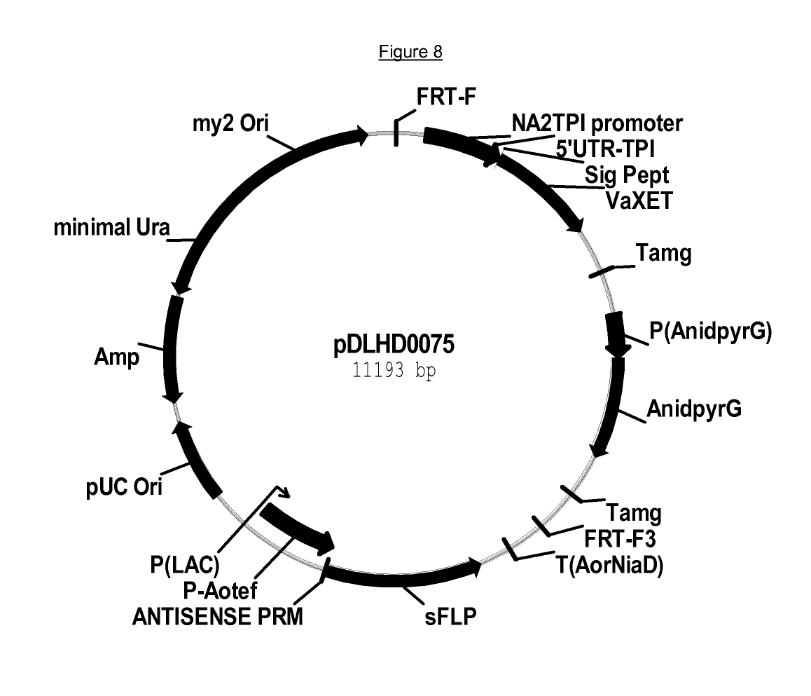

Herein a codon-optimized wild-type Vigna angularis xyloglucan endotransglycosylase 16 (VaXET16) cDNA was cloned into the A. oryzae Flp/FRT shuttle vector pDAu571 (FIG. 5) by yeast recombinational cloning, resulting in vector pDLHD0075 (FIG. 8).

Expression vector pDLHD0075 was constructed to contain the E. coli pUC origin of replication, E. coli beta-lactamase (ampR) selectable marker, URA3 yeast selectable marker, yeast 2 micron origin of replication, NA2-tpi promoter, codon-optimized VaXET16 open reading frame (SEQ ID NO:31 for the cDNA sequence and SEQ ID NO:32 for the amino acid sequence), Aspergillus niger glucoamylase terminator, Aspergillus nidulans pyrG selection marker, Saccharomyces cerevisiae 2 μm flippase ORF between the Aspergillus oryzae TEF1 promoter and Aspergillus oryzae NiaD terminator, and Saccharomyces cerevisiae 2 μm flippase recognition targets FRT-F and FRT-F3.

Plasmid pDLHD0075 was generated by combining four DNA fragments using yeast recombinational cloning: Fragment 1 contained the flippase expression cassette, FRT-F3, and AMG terminator from pDAU571 and flanking sequences with homology to fragments 4 and 2. Fragment 2 contained the E. coli pUC origin of replication, E. coli beta-lactamase (ampR) selectable marker, URA3 yeast selectable marker, yeast 2 micron origin of replication from pDLHD0044, and flanking sequences with homology to fragments 1 and 3. Fragment 3 contained the NA2-tpi promoter, the VaXET16 codon-optimized gene from pDLHD0044, and flanking sequences with homology to fragments 2 and 4. Fragment 4 contained the A. niger amyloglucosidase terminator sequence (AMG terminator) and Aspergillus nidulans orotidine-5′-phosphate decarboxylase gene (pyrG) as a selectable marker from pDAU571, and flanking sequences with homology to fragments 3 and 1.

Fragment 1 was amplified using primer 615726 (sense) and primer 615728 (antisense) shown below. These primers were designed to contain flanking regions of sequence homology to fragments 4 and 2, respectively (lower case), for ligation-free cloning between the PCR fragments.

Primer 615726 (sense):

(SEQ ID NO: 33)

accgggaggaaggctggaaaGCTTACGAGAAAAGAGTTGGACTTTGAGGG
Primer 615728 (antisense):

(SEQ ID NO: 34)

tgagcgaggaagcggAAGAGCGCCCAATACGCAAACCGCC

Fragment 1 was amplified by PCR in a reaction composed of 10 ng of pDAU571, 0.5 μl of PHUSION® DNA Polymerase, 20 pmol of primer 615726, 20 pmol of primer 615728, 1 μl of 10 mM dNTPs, 10 μl of 5× PHUSION® HF buffer, and 35.5 μl of water. The reaction was incubated in an EPPENDORF® MASTERCYCLER® thermocycler programmed for 1 cycle at 98° C. for 30 seconds; and 30 cycles each at 98° C. for 10 seconds, 60° C. for 10 seconds, and 72° C. for 120 seconds. The resulting 3.3 kb PCR product (fragment 1) was treated with 1 μl of Dpn I to remove plasmid template DNA. The Dpn I was added directly to the PCR reaction tube, mixed well, and incubated at 37° C. for 60 minutes.

Fragment 2 was amplified using primer 615729 (sense) and primer 615731 (antisense) shown below. These primers were designed to contain flanking regions of sequence homology to fragments 1 and 3, respectively (lower case), for ligation-free cloning between the PCR fragments.

Primer 615729 (sense):

(SEQ ID NO: 35)

tgcgtattgggcgctcttCCGCTTCCTCGCTCACTGACTC
Primer 615731 (antisense):

(SEQ ID NO: 36)

tatactttctagagaataggaactcggaataggaacttcaaGGAACAACA
CTCAACCCTATCTCGGTC

Fragment 2 was amplified by PCR in a reaction composed of 10 ng of pDLHD0044, 0.5 μl of PHUSION® DNA Polymerase, 20 pmol of primer 615729, 20 pmol of primer 615731, 1 μl of 10 mM dNTPs, 10 μl of 5× PHUSION® HF buffer, and 35.5 μl of water. The reaction was incubated in an EPPENDORF® MASTERCYCLER® thermocycler programmed for 1 cycle at 98° C. for 30 seconds; and 30 cycles each at 98° C. for 10 seconds, 60° C. for 10 seconds, and 72° C. for 120 seconds. The resulting 4.2 kb PCR product (fragment 2) was treated with 1 μl of Dpn I to remove plasmid template DNA. The Dpn I was added directly to the PCR reaction tube, mixed well, and incubated at 37° C. for 60 minutes.

Fragment 3 was amplified using primer 615730 (sense) and primer 615611 (antisense) shown below. These primers were designed to contain flanking regions of sequence homology to fragments 2 and 4, respectively (lower case), for ligation-free cloning between the PCR fragments.

Primer 615730 (sense):

(SEQ ID NO: 37)

tccgagttcctattctctagaaagtataggaacttcGCATTTATCAGGGT
TATTGTCTCATGAGCGG
Primer 615611 (antisense):

(SEQ ID NO: 38)

tctagatctcgagtcaGATGTCCCTATCGCGTGTACACTCG

Fragment 3 was amplified by PCR in a reaction composed of 10 ng of pDLHD0044, 0.5 μl of PHUSION® DNA Polymerase, 20 pmol of primer 615730, 20 pmol of primer 615611, 1 μl of 10 mM dNTPs, 10 μl of 5× PHUSION® HF buffer, and 35.5 μl of water. The reaction was incubated in an EPPENDORF® MASTERCYCLER® thermocycler programmed for 1 cycle at 98° C. for 30 seconds; and 30 cycles each at 98° C. for 10 seconds, 60° C. for 10 seconds, and 72° C. for 120 seconds. The resulting 1.7 kb PCR product (fragment 3) was treated with 1 μl of Dpn I to remove plasmid template DNA. The Dpn I was added directly to the PCR reaction tube, mixed well, and incubated at 37° C. for 60 minutes.

Fragment 4 was amplified using primer 615610 (sense) and primer 615727 (antisense) shown below. These primers were designed to contain flanking regions of sequence homology to fragments 3 and 1, respectively (lower case), for ligation-free cloning between the PCR fragments.

Primer 615610 (sense):

(SEQ ID NO: 39)

acacgcgatagggacatcTGACTCGAGATCTAGAGGGTGACTGAC
Primer 615727 (antisense):

(SEQ ID NO: 40)

aactcttttctcgtaagcTTTCCAGCCTTCCTCCCGGTAC

Fragment 4 was amplified by PCR in a reaction composed of 10 ng of pDAU571, 0.5 μl of PHUSION® DNA Polymerase, 20 pmol of primer 615610, 20 pmol of primer 615727, 1 μl of 10 mM dNTPs, 10 μl of 5× PHUSION® HF buffer, and 35.5 μl of water. The reaction was incubated in an EPPENDORF® MASTERCYCLER® thermocycler programmed for 1 cycle at 98° C. for 30 seconds; and 30 cycles each at 98° C. for 10 seconds, 60° C. for 10 seconds, and 72° C. for 120 seconds. The resulting 1.9 kb PCR product (fragment 4) was treated with 1 μl of Dpn I to remove plasmid template DNA. The Dpn I was added directly to the PCR reaction tube, mixed well, and incubated at 37° C. for 60 minutes.

The following procedure was used to combine the four PCR fragments using yeast homology-based recombinational cloning. A 10 μl aliquot of each of the PCR fragments was combined with 100 μg of single-stranded deoxyribonucleic acid from salmon testes (Sigma-Aldrich, St. Louis, Mo., USA), 100 μl of competent yeast cells of strain YNG318 (Saccharomyces cerevisiae ATCC 208973), and 600 μl of PLATE Buffer (Sigma Aldrich, St. Louis, Mo., USA), and mixed. The reaction was incubated at 30° C. for 30 minutes with shaking at 200 rpm. The reaction was then continued at 42° C. for 15 minutes with no shaking. The cells were pelleted by centrifugation at 5,000×g for 1 minute and the supernatant was discarded. The cell pellet was suspended in 200 μl of autoclaved water and split over two agar plates containing Synthetic Defined medium lacking uridine and incubated at 30° C. for three days. The yeast colonies were isolated from the plate using 1 ml of autoclaved water. The cells were pelleted by centrifugation at 13,000×g for 30 seconds and a 100 μl aliquot of glass beads were added to the tube. The cell and bead mixture was suspended in 250 μl of P1 buffer (QIAGEN Inc., Valencia, Calif., USA) and then vortexed for 1 minute to lyse the cells. The plasmid DNA was purified using a QIAPREP® Spin Miniprep Kit. A 3 μl aliquot of the plasmid DNA was then transformed into E. coli ONE SHOT® TOP10 electrocompetent cells according the manufacturer's instructions. Fifty μl of transformed cells were spread onto 2XYT plates supplemented with 100 μg of ampicillin per ml and incubated at 37° C. overnight. Transformants were each picked into 3 ml of LB medium supplemented with 100 μg of ampicillin per ml and grown overnight at 37° C. with shaking at 250 rpm. The plasmid DNA was purified from colonies using a QIAPREP® Spin Miniprep Kit. DNA sequencing with a 3130XL Genetic Analyzer was used to confirm the presence of each of the three fragments in a final plasmid designated plasmid pDLHD0075 (FIG. 8).

### Example 7: Confirmation of Wild-Type Vigna angularis Xyloglucan Endotransglycosylase 16 (VaXET16) Expression in Single-Copy in Aspergillus oryzae Screening Strain JaL1394

Aspergillus oryzae JaL1394 (disclosed in WO 2012/160093) was transformed with plasmid pDLHD0075 comprising the codon-optimized VaXET16 gene.

Approximately 10⁷ spores from A. oryzae JaL1394 were inoculated into 100 ml of YP+2% glucose medium supplemented with 10 mM uridine in a 500 ml shake flask and incubated at 28° C. and 110 rpm overnight. 10 ml of the overnight culture were filtered in a 125 ml sterile vacuum filter, and the mycelia were washed twice with 50 ml of 0.7 M KCl-20 mM CaCl₂. The remaining liquid was removed by vacuum filtration, leaving the mat on the filter. The mycelia were resuspended in 10 ml of 0.7 M KCl-20 mM CaCl₂ and transferred to a sterile 125 ml shake flask containing 20 mg of GLUCANEX® 200 G (Novozymes Switzerland AG, Neumatt, Switzerland) per ml and 0.2 mg of chitinase (Sigma-Aldrich, St. Louis, Mo., USA) per ml in 10 ml of 0.7 M KCl-20 mM CaCl₂. The mixture was incubated at 37° C. and 100 rpm for 30-90 minutes until protoplasts were generated from the mycelia. The protoplast mixture was filtered through a sterile funnel lined with MIRACLOTH® (Calbiochem, San Diego, Calif., USA) into a sterile 50 ml plastic centrifuge tube to remove mycelial debris. The debris on the MIRACLOTH® was washed thoroughly with 10 ml of 0.7 M KCl-20 mM CaCl₂, and centrifuged at 2500 rpm for 10 minutes at 20-23° C. The supernatant was removed and the protoplast pellet was resuspended in 20 ml of 1 M sorbitol-10 mM CaCl₂−10 mM Tris-HCl (pH 6.5). This step was repeated twice, and the final protoplast pellet was resuspended in 1 M sorbitol-10 mM CaCl₂−10 mM Tris-HCl (pH 6.5) to obtain a final protoplast concentration of 2×10⁷/ml.

Protoplasts were transformed by the addition of two μg of pDLHD0075 to the bottom of a sterile 12 ml plastic centrifuge tube. One hundred μl of protoplasts were added to the tube followed by 300 μl of 60% PEG-4000 in 10 mM CaCl₂-10 mM Tris-HCl (pH 6.5). The tube was mixed gently by hand and incubated at 37° C. for 30 minutes. Five ml of 1 M sorbitol-10 mM CaCl₂-10 mM Tris-HCl (pH 6.5) were added to the transformation and the mixture was transferred onto 150 mm Minimal medium agar plates. Transformation plates were incubated at 37° C. until transformants appeared.

Single transformants were picked to new Minimal medium agar plates and cultivated at 37° C. for four days until the transformants sporulated. Fresh spores were transferred to 48-well deep-well plates containing 2 ml of YP+2% maltodextrin medium, covered with a breathable seal, and grown for 4 days at 28° C. with no shaking. After 4 days growth the culture medium for each transformant was assayed for xyloglucan endotransglycosylase activity and for xyloglucan endotransglycosylase expression by SDS-PAGE as outlined below.

The activity assay demonstrated that the transformants produced active xyloglucan endotransglycosylase.

### Iodine Colorimetric Assay to Determine Xyloglucan Endotransglycosylase Activity

Xyloglucan endotransglycosylase activity was assayed using a modified version of an iodine colorimetric assay described by Sulova et al., 1995, Analytical Biochechemistry 229: 80-85. For each reaction, 5 μl of tamarind seed xyloglucan (Megazyme, Bray, UK) (5 mg/ml in water) were combined with 20 μl of xyloglucan oligomers (Megazyme, Bray, UK) (5 mg/ml in water), 10 μl of 400 mM sodium citrate pH 5.5, and dispensed into 96 well plates. Reactions were initiated by the addition of 5 μl of liquid culture broth to each well, and plates were incubated at 37° C. for 10 minutes. Reactions were quenched by the addition of 200 μl of a solution composed of 14% (w/v) Na₂SO₄, 0.2% KI, 0.1 M HCl, and 0.5% I₂, and incubated in the dark for 30 minutes prior to measuring the absorbance at 620 nm in a SPECTRAMAX® M5 spectrophotometer (Molecular Devices, Sunnyvale, Calif., USA).

SDS-PAGE was performed using a 8-16% CRITERION® Stain Free SDS-PAGE gel (Bio-Rad Laboratories, Inc., Hercules, Calif., USA), and imaging the gel with a Stain Free Imager (Bio-Rad Laboratories, Inc., Hercules, Calif., USA) using the following settings: 5-minute activation, automatic imaging exposure (intense bands), highlight saturated pixels=ON, color=Coomassie, and band detection, molecular weight analysis and reporting disabled. SDS-PAGE revealed a band of approximately 32 kDa for the wild-type VaXET16.

### Example 8: Construction and Identification of Improved Expression Variants of Vigna angularis Xyloglucan Endotransglycosylase 16 (VaXET16)

VaXET16 gene mutant libraries were constructed by site-saturation mutagenesis. The mutant libraries of the VaXET16 gene (each fragment of the library comprises a mutant VaXET16 gene plus Aspergillus nidulans orotidine 5′-phosphate decarboxylase pyrG selection marker and the FRT-F and FRT-F3 flippase recognition target sequences) were transformed into protoplasts of Aspergillus oryzae JaL1394 as described in the previous example along with the pDLHD0095 vector comprising the Saccharomyces cerevisiae 2 μm flippase ORF between the Aspergillus oryzae TEF1 promoter and Aspergillus oryzae niaD gene terminator. After 4 days of protoplast recovery at 37° C. on Minimal medium agar plates, single colonies were picked into individual wells of 48-well deep-well plates containing 2 ml of YP+2% maltodextran medium, covered with a breathable seal, and grown for 4 days at 28° C. with no shaking. After 4 days growth the liquid culture medium was assayed for xyloglucan endotransglycosylase activity as described in the previous example, and higher activity variants were scored as expression hits.

Individual mutant strains were spore purified and cultivated again to generate fresh broth for re-testing relative to A. oryzae JaL1394 strain expressing the wild-type VaXET16 using the codon-optimized gene. Broths were also analyzed by SDS-PAGE as described in the previous example for increased production of the xyloglucan endotransglycosylase protein product.

Relative improvements in expression yield over the parent gene in day 4 broths from 48-well deep-well plate cultivations for eleven characterized variants are shown in Table 1 below. SDS-PAGE analysis of the same broths demonstrated a VaXET band of increased intensity over wild-type VaXET for all variants, which correlated well with the relative improvements observed in the activity assay. The SDS-PAGE band for the wild-type VaXET and variants thereof was 32 kDa, except variants containing the N175S mutation had a band of approximately 37 kDa due to additional glycosylation.

TABLE 1
Relative Improvement Over Parent

| VaXET16 Variant | Iodine Colorimetric Assay |
|---|---|
| Wild-Type | 1.0 |
| A40G, N175S | 1.8 |
| A40G, F183I | 2.9 |
| A40G, I53A, N175S | 3.8 |
| A40G, N175S, F183I | 1.1 |
| I10A, I53A, E102G | 1.1 |
| P30E, S51T, Y60S, T99N | 2.3 |
| A40G, E102G, Q117E | 3.1 |
| A40G, T99E, E102G, K130R | 1.2 |
| N175G, S280G | 2.7 |
| N175Q, A254E, S280E | 1.2 |
| I53V, R136W, Y157H, Y162C, N175S | 2.9 |

### Example 9: Fermentation-Scale Confirmation of Improved Expression of Vigna angularis Xyloglucan Endotransglycosylase 16 Variant (VaXET16) Genes in Aspergillus oryzae

A fermentation process was used to express the VaXET16 variants, A40G+I53A+N175S and A40G+F183I, relative to the wild-type VaXET16.

Shake flask medium was composed of 50 g of sucrose, 10 g of KH₂PO₄, 0.5 g of CaCl₂, 2 g of MgSO₄.7H₂O, 2 g of K₂SO₄, 2 g of urea, 10 g of yeast extract, 2 g of citric acid, 0.5 ml of trace metals solution, and deionized water to 1 liter. Trace metals solution was composed of 13.8 g of FeSO₄.7H₂O, 14.3 g of ZnSO₄.7H₂O, 8.5 g of MnSO₄.H₂O, 2.5 g of CuSO₄.5H₂O, 3 g of citric acid, and deionized water to 1 liter.

One hundred ml of shake flask medium were added to a 500 ml shake flask. The shake flask was inoculated with two plugs from a solid plate culture and incubated at 34° C. on an orbital shaker at 200 rpm for 24 hours. Fifty ml of the shake flask broth were used to inoculate a 3 liter fermentation vessel.

Fermentation batch medium was composed per liter of 10 g of yeast extract, 24 g of sucrose, 5 g of (NH₄)₂SO₄, 2 g of KH₂PO₄, 0.5 g of CaCl₂.2H₂O, 2 g of MgSO₄.7H₂O, 1 g of citric acid, 2 g of K₂SO₄, 0.5 ml of anti-foam, and 0.5 ml of trace metals solution. Trace metals solution was composed per liter of 13.8 g of FeSO₄.7H₂O, 14.3 g of ZnSO₄.7H₂O, 8.5 g of MnSO₄.H₂O, 2.5 g of CuSO₄.5H₂O, and 3 g of citric acid. Fermentation feed medium was composed of maltose.

A total of 1.8 liters of the fermentation batch medium was added to a three liter glass jacketed fermentor. Fermentation feed medium was dosed at a rate of 0 to 4.4 g/l/hr. The fermentation vessel was maintained at a temperature of 34° C. and pH was controlled to a set-point of 6.1+/−0.1. Air was added to the vessel at a rate of 1 vvm and the broth was agitated by Rushton impeller rotating at 1100 to 1300 rpm. Samples were taken on days 3, 4, 5, 6, and 7 of the fermentation run and centrifuged at 3000×g to remove the biomass. The supernatants were sterile filtered and stored at 5 to 10° C.

VaXET16 variant expression levels were determined relative to the wild-type codon-optimized gene by the fluorescence polarization assay outlined below and by SDS-PAGE analysis (see above).

### Fluorescence Polarization Assay for Xyloglucan Endotransglycosylation Activity

First, fluorescein isothiocyanate-labeled xyloglucan oligomers (FITC-XGOs) were generated by reductive amination of the reducing ends of xyloglucan oligomers according to the procedure described by Zhou et al., 2006, Biocatalysis and Biotransformation 24: 107-120), followed by conjugation of the amino groups of the XGOs to fluorescein isothiocyanate isomer I (Sigma Aldrich, St. Louis, Mo., USA) in 100 mM sodium bicarbonate pH 9.0 for 24 hours at room temperature. Conjugation reaction products were concentrated to dryness in vacuo, dissolved in 0.5 ml of deionized water, and purified by silica gel chromatography, eluting with a gradient from 100:0:0.04 to 70:30:1 acetonitrile:water:acetic acid as mobile phase. Purity and product identity were confirmed by evaporating the buffer, dissolving in D₂O (Sigma Aldrich, St. Louis, Mo., USA), and analysis by ¹H NMR using a Varian 400 MHz MercuryVx (Agilent, Santa Clara, Calif., USA). Dried FITC-XGOs were stored at −20° C. in the dark, and were desiccated during thaw.

One mg of FITC-XGOs was incubated with 1 mg of tamarind seed xyloglucan (Megazyme, Bray, United Kingdom) and 18 mg of VaXET16 per ml of 20 mM sodium citrate pH 5.0 in 200 μl reactions for at least 30 minutes Sample mixtures were pooled and precipitated by addition of ice cold ethanol to 80% (v/v) final concentration and incubation at 4° C. overnight. Precipitated fluorescein isothiocyanate-labeled xyloglucan (FITC-XG) was recovered by centrifugation at 3000 rpm using a LEGEND™ RT Plus centrifuge (Thermo Scientific, Waltham, Mass., USA) decanting of the ethanol, and drying at room temperature for 24 hours. FITC-XG was dissolved in a minimum volume of deionized water until dissolved and stored at −20° C. Frozen FITC-XG was thawed and lyophilized overnight. The lyophilized powder was dissolved in 500 μl of deionized water and quantified by absorbance at 488 nm.

A large scale batch of FITC-XG was prepared in the following manner. A 7.9 mg per ml solution of FITC-XGOs was prepared in deionized water. Forty ml of 10 mg of tamarind seed xyloglucan (Megazyme, Bray, UK) per ml of deionized water, 452 ml of 7.9 mg of FITC-XGOs per ml of deionized water, 2 ml of 400 mM sodium citrate pH 5.5, and 1.2 ml of 1.4 mg of VaXET16 per ml of 20 mM sodium citrate pH 5.5 were mixed thoroughly and incubated overnight at room temperature. Following overnight incubation, FITC-XG was precipitated by addition of ice cold ethanol to a final volume of 110 ml, mixed thoroughly, and incubated at 4° C. overnight. The precipitated FITC-XG was washed with water and then transferred to Erlenmeyer bulbs. Residual water and ethanol were removed by evaporation using an EZ-2 Elite evaporator (SP Scientific/Genevac, Stone Ridge, N.Y., USA) for 4 hours. Dried samples were dissolved in water, and the volume was adjusted to 48 ml with deionized water to generate a final FITC-XG concentration of 5 mg per ml with an expected average molecular weight of 100 kDa.

Xyloglucan endotransglycosylation activity was assessed using the following assay. Reactions of 200 μl containing 1 mg of tamarind seed xyloglucan per ml, 0.01 mg/ml FITC-XGOs prepared as described above, and 10 μl of appropriately diluted XET were incubated for 10 minutes at 25° C. in 20 mM sodium citrate pH 5.5 in opaque 96-well microtiter plates. Fluorescence polarization was monitored continuously over this time period, using a SPECTRAMAX® M5 microplate reader (Molecular Devices, Sunnyvale, Calif., USA) in top-read orientation with an excitation wavelength of 490 nm, an emission wavelength of 520 nm, a 495 cutoff filter in the excitation path, high precision (100 reads), and medium photomultiplier tube sensitivity. XET-dependent incorporation of fluorescent XGOs into non-fluorescent XG results in increasing fluorescence polarization over time. The slope of the linear regions of the polarization time progress curves was used to determine the activity.

Relative improvements in production yield over the parent gene for day 7 broths of the two variants relative to the wild-type VaXET16 are shown in Table 2 below. Variant A40G+I53A+N175S was produced in an amount that was 3.1× greater than the wild-type VaXET16, while variant A40G+F183I was produced in an amount that was 1.2× greater than the wild-type VaXET16. SDS-PAGE analysis of the same broths showed a VaXET band of increased intensity over wild-type VaXET for both variants which correlated well with the relative improvements observed in the activity assay. SDS-PAGE analysis of the samples taken on days 3, 4, 5, 6, and 7 showed increased production of VaXET and each variant day by day with day 7 the strongest.

TABLE 2
Relative Improvement Over Parent

| VaXET16 Variant | Fluorescence Polarization Assay |
|---|---|
| Wild-Type | 1.0 |
| A40G, I53A, N175S | 3.1 |
| A40G, F183I | 1.2 |

The invention described and claimed herein is not to be limited in scope by the specific aspects herein disclosed, since these aspects are intended as illustrations of several aspects of the invention. Any equivalent aspects are intended to be within the scope of this invention. Indeed, various modifications of the invention in addition to those shown and described herein will become apparent to those skilled in the art from the foregoing description. Such modifications are also intended to fall within the scope of the appended claims. In the case of conflict, the present disclosure including definitions will control.

### Example 10: Trichoderma reesei Protoplast Generation and Transformation

Protoplast preparation and transformation were performed using the following protocol based on Penttila et al., 1987, Gene 61: 155-164. A Trichoderma reesei strain was cultivated in 25 ml of YP medium supplemented with 2% (w/v) glucose and 10 mM uridine at 27° C. for 17 hours with gentle agitation at 90 rpm. Mycelia were collected by filtration using a Vacuum Driven Disposable Filtration System (Millipore) and washed twice with deionized water and twice with 1.2 M sorbitol. Protoplasts were generated by suspending the washed mycelia in 20 ml of 1.2 M sorbitol containing 15 mg of GLUCANEX® 200 G (Novozymes NS, Bagsvaerd, Denmark) per ml and 0.36 units of chitinase (Sigma Chemical Co.) per ml for 15-25 minutes at 34° C. with gentle shaking at 90 rpm. Protoplasts were collected by centrifuging for 7 minutes at 400×g and washed twice with cold 1.2 M sorbitol. The protoplasts were counted using a haemacytometer and resuspended to a final concentration of 1×10⁸ protoplasts/ml in STC.

Approximately 1-10 μg of DNA were added to 100 μl of the protoplast solution and mixed gently. PEG buffer (250 μl) was then added, and the transformation reaction was mixed and incubated at 34° C. for 30 minutes. STC (3 ml) was then added to the transformation reaction and mixed. The transformation reaction was then spread onto COVE plates for amdS selection. The plates were incubated at 28° C. for 6-11 days.

For transformation requiring hygromycin selection, the reaction mix in STC was spread onto PDA plates supplemented with 1 M sucrose. After incubation at 28° C. for 16 hours, 20 ml of overlay PDA medium supplemented with 35 μg of hygromycin B per ml were added to each plate. The plates were incubated at 28° C. for 4-7 days.

### Example 11: Genomic DNA Extraction from Trichoderma reesei Strains

A Trichoderma reesei strain was grown in 50 ml of YP medium supplemented with 2% glucose (w/v) in a 250 ml baffled shake flask at 28° C. for 2 days with agitation at 200 rpm. Mycelia from each cultivation were collected using a MIRACLOTH® (EMD Chemicals Inc.) lined funnel, squeeze-dried, and frozen under liquid nitrogen. The frozen mycelia were transferred to a pre-chilled mortar and pestle. Each mycelia preparation was ground into a fine powder and kept frozen with liquid nitrogen. A total of 1-2 g of powder was transferred to a 50 ml tube and genomic DNA was extracted from the ground mycelial powder using a DNEASY® Plant Maxi Kit (QIAGEN Inc.). Five ml of Buffer AP1 (QIAGEN Inc.) pre-heated to 65° C. were added to the 50 ml tube followed by 10 μl of a RNase A 100 mg/ml stock solution (QIAGEN Inc.), and incubated for 2-3 hours at 65° C. A total of 1.8 ml of AP2 Buffer (QIAGEN Inc.) was added and the tube was incubated on ice for 5 minutes followed by centrifugation at 3000-5000×g for 5 minutes in a LEGEND™ RT swinging bucket centrifuge (Thermo Fisher Scientific Inc.). The supernatant was transferred to a QIAShredder™ Maxi Spin Column (QIAGEN Inc.) placed in a 50 ml collection tube, and centrifuged at 3000-5000×g at room temperature for 5 minutes (15-25° C.) in a swing-out rotor. The flow-through in the collection tube was transferred, without disturbing the pellet, into a new 50 ml tube. A 1.5 ml volume of Buffer AP3/E (QIAGEN Inc.) was added to the cleared lysate, and mixed immediately by vortexing. The sample (maximum 15 ml), including any precipitate that may have formed, was pipetted into a DNEASY® Maxi Spin Column (QIAGEN Inc.) placed in a 50 ml collection tube and centrifuged at 3000-5000×g for 5 minutes at room temperature (15-20° C.) in a swing-out rotor. The flow-through was discarded. Twelve ml of Buffer AW (QIAGEN Inc.) were added to the DNEASY® Maxi Spin Column, and centrifuged at 3000-5000×g for 10 minutes to dry the membrane. The flow-through and collection tube were discarded. The DNEASY® Maxi Spin Column was transferred to a new 50 ml tube. The DNA was eluted by adding 1-1.5 ml of Kit-supplied buffer AE, pre-heated to 65° C., directly onto the DNEASY® Maxi Spin Column membrane, incubating at room temperature for 5 minutes (15-25° C.), and then centrifuging at 3000-5000×g for 5 minutes. The concentration and purity of the genomic DNA was determined by measuring the absorbance at 260 nm and 280 nm.

### Example 12: Southern Blot Analysis of Transformants

Two μg of genomic DNA from each transformant were digested with selected restriction enzyme(s). The digestions were submitted to 0.7-0.8% agarose gel electrophoresis in TAE buffer and blotted onto a HYBOND® N+ blotting membrane (GE Healthcare Life Sciences) or a NYTRAN® SuperCharge membrane (Schleicher & Schuell BioScience) using a TURBOBLOTTER® (GE Healthcare Life Sciences) for approximately 1-2 hours. The membrane was hybridized with a digoxigenin-labeled gene-specific or site-specific probe, which was synthesized by PCR using a PCR DIG Probe Synthesis Kit (Roche Applied Science Corp.).

The probe was boiled for 5 minutes, chilled on ice for 2 minutes, and added to 10 ml of DIG Easy Hyb to produce the hybridization solution. Hybridization was performed in DIG Easy Hyb buffer at 42° C. for 15-17 hours. The membrane was then washed in 2×SSC plus 0.1% SDS at room temperature for 5 minutes followed by two washes in 0.5×SSC plus 0.1% SDS each at 65° C. for 15 minutes. After one wash in 1× Blocking Solution (Roche Applied Science Corp.) for a minimum of one hour, the membrane was incubated with 50 ml of 1× Blocking Solution containing 3.75 U of anti-digoxigenin-AP Fab fragments (Roche Applied Science Corp.) for 10 minutes, followed by two washes in 1× Washing Solution (Roche Applied Science Corp.). The CDP-STAR® ready-to-use reagent (disodium 2-chloro-5-(4-methoxyspiro (1,2-dioxetane-3,2′-(5′-chloro)tricyclo[3.3.1.1^{3,7}]decan}-4-yl)-1-phenyl phosphate; Roche Applied Science Corp.) was then applied to the membrane and the probe-target hybrids were detected by autoradiography.

### Example 13: Construction of FLP/FRT Integration Plasmid pJfyS148B

The FRT-F3 site was first inserted into plasmid pSMai155 (WO 05/074647) using a QUICKCHANGE® II XL Site-Directed Mutagenesis Kit (Agilent Technologies) with the mutagenic insertion primers shown below.

Forward primer:

(SEQ ID NO: 41)

5′-CGAATTCTGCATTGAAGTTCCTATTCCGAGTTCCTATTCTTCAAATA
GTATAGGAACTTCAGATATCCATCACACTGGCG-3′
Reverse primer:

(SEQ ID NO: 42)

5′-GCCAGTGTGATGGATATCTGAAGTTCCTATACTATTTGAAGAATAGG
AACTCGGAATAGGAACTTCAATGCAGAATTCGC-3′

The mutagenic PCR contained 10 ng of pSMai155, 200 μM dNTPs, 125 ng of each primer, 1× QUICKCHANGE® Reaction Buffer (Agilent Technologies), 3 μl of QUIKSOLUTION® reagent (Agilent Technologies), and 2.5 units of Pfu Ultra High Fidelity DNA polymerase (Agilent Technologies) in a final volume of 50 μl. The PCR was performed in a thermocycler programmed for 1 cycle at 95° C. for 1 minute; 18 cycles each at 95° C. for 50 seconds, 60° C. for 50 seconds, and 68° C. for 40 seconds; and 1 cycle at 68° C. for 7 minutes. One μl of Kit-supplied Dpn I was added and the reaction was incubated at 37° C. for 1 hour. Two μl of the Dpn I-treated reaction were added to 45 μl of Kit-supplied XL10-Gold Ultracompetent E. coli cells (Agilent Technologies) in a 14 ml tube and incubated on ice for 30 minutes. The tube was incubated at 42° C. for 30 seconds after which 0.5 ml of SOC medium was added. The tube was then incubated at 37° C. with agitation at 200 rpm for 1 hour after which 250 μl each were plated onto 2×150 mm 2XYT plus ampicillin plates and incubated at 37° C. overnight. E. coli transformants were inoculated into 3 ml of LB+Amp medium in 14 ml tubes and incubated overnight at 37° C. with agitation at 200 rpm. Plasmid DNA was isolated using a BIOROBOT® 9600 (QIAGEN Inc.). The insert was confirmed by DNA sequencing. One transformant was identified as containing the desired sequence insertion corresponding to the FRT-F3 site and the plasmid was designated pJfyS148A.

The FRT-F sequence was then inserted into plasmid pJfyS148A using an IN-FUSION® Advantage PCR Cloning Kit (Clontech Laboratories, Inc.). The FRT-F site was first amplified by PCR from plasmid pRika147 (WO 2012/120093) using the primers shown below.

Forward primer:

(SEQ ID NO: 43)

5′-ATATCCATCACACTGGCGGCCGCTCAACTCTCTCCTCTAGGTTGAAG
TTCCTATTCCGAGTTC-3′
Reverse primer:

(SEQ ID NO: 44)

5′-AGGATGCATGCTCGAGCATGCACTAGCTAGTTGAAGTTCCTATA
C-3′

The PCR was composed of 20 ng of pRika147, 200 μM dNTPs, 0.4 μM primers, 1× PHUSION® Reaction Buffer (Thermo Fisher Scientific, Inc.), and 2 units of PHUSION® High Fidelity DNA polymerase (Thermo Fisher Scientific, Inc.) in a final volume of 50 μl. The reaction was performed in a thermocycler programmed for 1 cycle at 95° C. for 2 minutes; 30 cycles each at 95° C. for 25 seconds, 50° C. for 25 seconds, and 72° C. for 40 seconds; and 1 cycle at 72° C. for 7 minutes. The completed PCR was submitted to 2% agarose gel electrophoresis in TAE buffer where a 0.1 kb fragment was excised from the gel and agarose was extracted using a MINELUTE® Gel Extraction Kit (QIAGEN Inc.). Briefly, 3 volumes of Kit-supplied buffer QG were added to the gel slice and dissolved at 50° C. for approximately 10 minutes. The dissolved gel slice was applied to a Kit-supplied spin column by transferring to the column and centrifuging at 13,000 rpm for 1 minute. The column was washed with 750 μl of Kit-supplied buffer PE and then re-centrifuged. DNA was eluted with 10 μl of Kit-supplied buffer EB.

The 0.1 kb PCR product was inserted into Sal I-digested pJfyS148A using an IN-FUSION® Advantage PCR Cloning Kit. The reaction was composed of 1× IN-FUSION® Reaction Buffer, 125 ng of pJfyS147A, 20 ng of FRT-F PCR product, and 1 μl of IN-FUSION® Enzyme in a 10 μl reaction volume. The reaction was incubated at 50° C. for 15 minutes. Then 40 μl of TE were added to the reaction and 2 μl were transformed into ONE SHOT® TOP10 E. coli chemically competent cells (Invitrogen Corp.) by addition to a single use tube containing the competent cells and incubating the cells on ice for 5 minutes. The tube was incubated at 42° C. for 30 seconds after which 250 μl of SOC medium were added. The tube was then incubated at 37° C. with agitation at 200 rpm for 1 hour and 250 μl were transferred to a 150 mm 2XYT plus ampicillin plate and incubated overnight at 37° C. E. coli transformants were inoculated into 3 ml of LB+Amp medium in 14 ml tubes and incubated overnight at 37° C. with agitation at 200 rpm. Plasmid DNA from E. coli transformants was isolated using a BIOROBOT® 9600 (QIAGEN Inc.). The insert was confirmed by DNA sequencing. One transformant was identified as containing the insert with no PCR errors and the plasmid was designated pJfyS148B.

### Example 14: Construction of Plasmid pJfyS156

To construct plasmid pJfyS156 the cbh2 gene promoter and flippase gene were PCR amplified using gene-specific primers shown below and inserted into plasmid pJfyS148B.

### Flippase

Forward primer:

(SEQ ID NO: 45)

5′-CACCCTCTGTGTATTGCACCATGCCCCAGTTCGATATCCTCTGC
A-3′
Reverse primer:

(SEQ ID NO: 46)

5′-AAACTCTAGGATGCATGCAAGTGAGGCTATTGCCTATCAGCTC-3′

cbh2 Gene Promoter

Forward primer:

(SEQ ID NO: 47)

5′-CATCACACTGGCGGCCGCGAATTCTAGGCTAGGTATGC-3′
Reverse primer:

(SEQ ID NO: 48)

5′-GGTGCAATACACAGAGGGTG-3′

The PCR was composed of 20 ng of template pRiKa147 for the flippase PCR or 150 ng of T. reesei 981-O-8 genomic DNA for the cbh2 promoter PCR, 200 μM dNTPs, 0.4 μM primers, 1× PHUSION® Reaction Buffer, and 2 units of PHUSION® High Fidelity DNA polymerase in a final volume of 50 μl. The reactions were performed in a thermocycler programmed for 1 cycle at 95° C. for 2 minutes; 30 cycles each at 95° C. for 30 seconds, 57° C. for 30 seconds, and 72° C. for 2 minutes; and 1 cycle at 72° C. for 7 minutes. The completed PCRs were submitted to 1% agarose gel electrophoresis in TAE buffer where 1.2 and 0.6 kb bands, corresponding to the coding region for the S. cerevisiae flippase gene and the niaD terminator and cbh2 gene promoter, respectively, were excised from the gels and agarose was extracted using a Nucleospin® Extract II Kit (Macherey Nagel, Bethlehem, Pa., USA). Three volumes of Kit-supplied NT buffer were added to the gel slice and the sample was heated at 50° C. for 10 minutes. The entire solution was transferred to a Kit-supplied centrifugal column. The column was centrifuged at 13,000 rpm for 1 minute, and washed with Kit-supplied wash buffer NT3 and re-centrifuged. DNA was eluted with 30 μl of Kit-supplied elution buffer NE and centrifuged at 13,000 rpm for 1 minute.

The cbh2 gene promoter and flippase coding sequence were inserted in a single step into Xho I-linearized pJfyS148B using an IN-FUSION® Advantage PCR Cloning Kit. The reaction was composed of 1× IN-FUSION® Reaction Buffer, 180 ng of Xho I-linearized pJfyS148B, 100 ng of the 0.6 kb cbh2 promoter PCR product, 240 ng of the 1.2 kb flippase PCR product, and 1 μl of IN-FUSION® Enzyme in a 10 μl reaction volume. The reaction was incubated for 15 minutes at 37° C. and then 15 minutes at 50° C. Then 40 μl of TE were added to the reaction and 2 μl were transformed into ONE SHOT® TOP10 E. coli chemically competent cells according to Example 14. E. coli transformants were inoculated into 3 ml of LB+Amp medium in 14 ml tubes and incubated overnight at 37° C. with agitation at 200 rpm. Plasmid DNA was isolated using a BIOROBOT® 9600. The insert was confirmed by DNA sequencing. One transformant was identified as containing the inserts with no PCR errors and the plasmid was designated pJfyS155.

An A. fumigatus beta-glucosidase gene was amplified by PCR from pEJG107 (WO 05/047499) using the primers shown below.

Forward primer:

(SEQ ID NO: 49)

5′-ACCGCGGACTGCGCACCATGAGATTCGGTTGGCTCGAGG-3′
Reverse primer:

(SEQ ID NO: 50)

5′-TTCGCCACGGAGCTTACTAGTAGACACGGGGCAGAGGC-3′

The PCR was composed of 20 ng of pEJG107, 200 μM dNTPs, 0.4 μM primers, 1× PHUSION® Reaction Buffer, and 2 units of PHUSION® High Fidelity DNA polymerase in a final volume of 50 μl. The reaction was performed in a thermocycler programmed for 1 cycle at 95° C. for 2 minutes; 25 cycles each at 95° C. for 30 seconds, 57° C. for 30 seconds, and 72° C. for 2 minutes; and 1 cycle at 72° C. for 7 minutes. The completed PCR was submitted to 1% agarose gel electrophoresis in TAE buffer where a 3 kb band was excised from the gel and agarose was extracted using a MINELUTE® Gel Extraction Kit (Example 13).

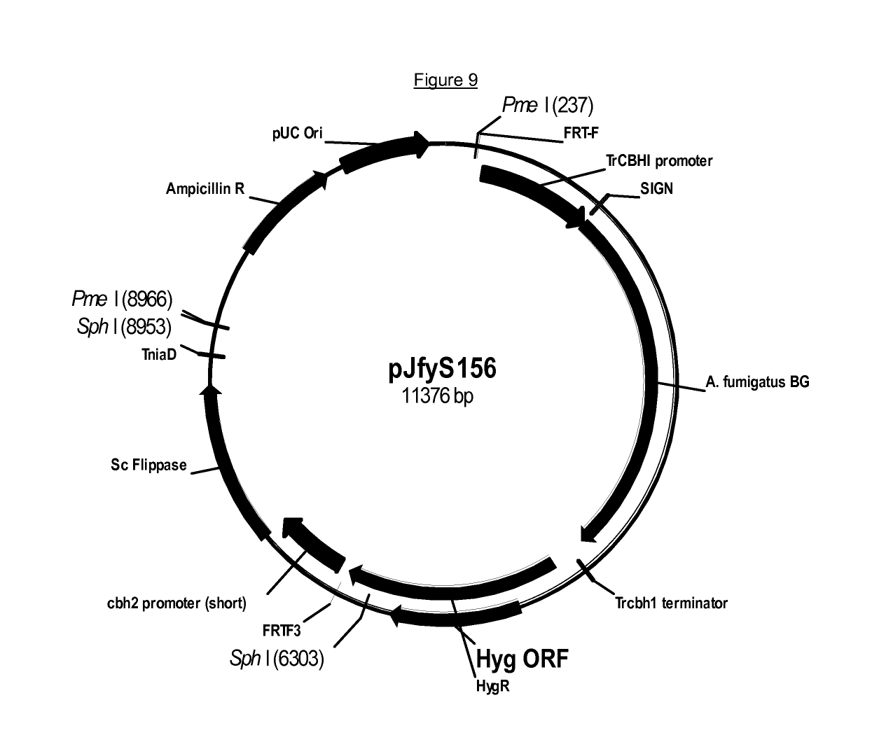

The A. fumigatus beta-glucosidase PCR product was inserted into Nco I/Pac I-digested pJfyS155 using an IN-FUSION® Advantage PCR Cloning Kit. The reaction was composed of 150 ng of Nco I/Pac I-digested pJfyS155, 100 ng of the A. fumigatus beta-glucosidase PCR product, 1× IN-FUSION® Advantage Buffer, and 1 μl of IN-FUSION® Enzyme in a 10 μl reaction volume. The reaction was incubated for 15 minutes at 37° C. and then 15 minutes at 50° C. Then 40 μl of TE were added to the reaction and 2 μl were transformed into ONE SHOT® TOP10 E. coli chemically competent cells according to Example 13. Plasmid DNA from E. coli transformants was isolated using a BIOROBOT® 9600. The insert was confirmed by DNA sequencing. One transformant was identified as containing the inserts with no PCR errors and the plasmid was designated pJfyS156 (FIG. 9).

### Example 15: Construction of Plasmid pDM313

A 0.38 kb PCR fragment containing a portion of the hpt marker, the FRT-F3 site, and a portion of the T. reesei gpdA promoter was amplified from pJfyS156 using the primers shown below.

Forward primer:

(SEQ ID NO: 51)

5′-CGTGTTTCTTCCCATTCGCATGCGACCTCGTGGTCATTGAC-3′
Reverse primer:

(SEQ ID NO: 52)

5′-GCTTTGACGTTACATTGACGTACTTATAAGCGGCCGCCAGTGTGATG
GA-3′

The PCR was composed of 50 picomoles of each of the primers, 100 ng of pJfyS156 DNA, 1× PHUSION™ High-Fidelity Hot Start DNA Polymerase buffer (Thermo Fisher Scientific, Inc.), 1 μl of a 10 mM blend of dNTPs, and 1 unit of PHUSION™ High-Fidelity Hot Start DNA Polymerase (Thermo Fisher Scientific, Inc.) in a final volume of 50 μl. The reaction was performed in a thermocycler programmed for 1 cycle at 98° C. for 2 minutes; 34 cycles each at 98° C. for 15 seconds, 59° C. for 30 seconds, and 72° C. for 1 minute; and 1 cycle at 72° C. for 10 minutes.

A 1.0 kb PCR fragment containing the T. reesei gpdA promoter was amplified from T. reesei RutC30 genomic DNA using the primers shown below.

Forward primer:

(SEQ ID NO: 53)

5′-TCCATCACACTGGCGGCCGCTTATAAGTACGTCAATGTAACGTCAAA
GC-3′
Reverse primer:

(SEQ ID NO: 54)

5′-TGCAGAGGATATCGAACTGGGGCATTTTGTATCTGCGAATTGAGCTT
G-3′

The PCR was composed of 50 picomoles of each of the primers, 100 ng of T. reesei RutC30 genomic DNA, 1× PHUSION™ High-Fidelity Hot Start DNA Polymerase buffer, 1 μl of a 10 mM blend of dNTPs, and 1 unit of PHUSION™ High-Fidelity Hot Start DNA Polymerase in a final volume of 50 μl. The reaction was performed in a thermocycler programmed for 1 cycle at 98° C. for 2 minutes; 34 cycles each at 98° C. for 15 seconds, 59° C. for 30 seconds, and 72° C. for 1 minute; and 1 cycle at 72° C. for 10 minutes.

A 1.8 kb PCR fragment containing the coding region for the S. cerevisiae flippase gene and the niaD terminator was amplified from plasmid pJfyS156 using the primers shown below.

Forward primer:

(SEQ ID NO: 55)

5′-CAAGCTCAATTCGCAGATACAAAATGCCCCAGTTCGATATCCTCTGC
A-3′
Reverse primer:

(SEQ ID NO: 56)

5′-GCTGTTTAAACTCTAGGATGCATGCAAGTGAGGCTATTGCC-3′

The PCR was composed of 50 picomoles of each of the primers, 100 ng of pJfyS156 DNA, 1× PHUSION™ High-Fidelity Hot Start DNA Polymerase buffer, 1 μl of a 10 mM blend of dNTPs, and 1 unit of PHUSION™ High-Fidelity Hot Start DNA Polymerase in a final volume of 50 μl. The reaction was performed in a thermocycler programmed for 1 cycle at 98° C. for 2 minutes; 34 cycles each at 98° C. for 15 seconds, 59° C. for 30 seconds, and 72° C. for 1 minute; and 1 cycle at 72° C. for 10 minutes.

The three completed PCRs described above were analysed by 0.8% agarose gel electrophoresis in TAE buffer where fragments of 0.38 kb, 1.0 kb, and 1.8 kb were confirmed. The PCR fragments in the original reactions were used as template for a SOE PCR described below.

The 0.38 kb and 1.0 kb PCR fragments were joined by SOE PCR using the primers shown below.

Forward primer:

(SEQ ID NO: 57)

5′-CGTGTTTCTTCCCATTCGCATGCGACCTCGTGGTCATTGAC-3′
Reverse primer:

(SEQ ID NO: 58)

5′-TGCAGAGGATATCGAACTGGGGCATTTTGTATCTGCGAATTGAGCTT
G-3′

The PCR was composed of 50 picomoles of each of the primers, 0.3 μl of the 0.38 kb fragment PCR, 0.6 μl of the 1.0 kb fragment PCR, 1× PHUSION™ High-Fidelity Hot Start DNA Polymerase buffer, 1 μl of a 10 mM blend of dNTPs, and 1 unit of PHUSION™ High-Fidelity Hot Start DNA Polymerase in a final volume of 50 μl. The reaction was performed in a thermocycler programmed for 1 cycle at 98° C. for 2 minutes; 34 cycles each at 98° C. for 15 seconds, 60° C. for 30 seconds, and 72° C. for 1.5 minutes, and 1 cycle at 72° C. for 10 minutes. Five SOE PCRs were performed and the reactions were combined. The 1.36 kb SOE PCR fragment and the 1.8 kb PCR fragment containing the flippase coding sequence and niaD terminator were separated by 0.8% agarose gel electrophoresis in TAE buffer where a 1.36 kb fragment and a 1.8 kb fragment were excised from the gel and extracted using a Nucleospin® Extract II Kit (Example 14).

Seventeen μg of pJfyS156 DNA were digested with Sph I and purified by 0.8% agarose gel electrophoresis in TAE buffer where an approximately 8.7 kb fragment was excised from the gel and extracted using a Nucleospin® Extract II Kit (Example 14).

The 1.36 kb SOE PCR fragment and the 1.8 kb PCR fragment, containing the flippase coding sequence and niaD terminator, were inserted into Sph I digested pJfyS156 using an IN-FUSION® HD Cloning Kit. The reaction was composed of 116 ng of Sph I digested pJfyS156, 57 ng of the 1.36 kb SOE PCR fragment, 69 ng of the 1.8 kb PCR fragment, and 2 μl of IN-FUSION® buffer with Enzyme in a 10 μl reaction volume. The reaction was incubated at 50° C. for 15 minutes and then chilled on ice. A 40 μl aliquot of TE was added. Two μl of the reaction transformed into ONE SHOT® TOP10 E. coli chemically competent cells according to Example 13. Plasmid DNA was isolated from the transformants using a BIOROBOT® 9600. Transformants were screened by restriction digestion with Nsi I which produced 2.1 kb, 2.6 kb, and 7 kb fragments. DNA sequencing of one clone verified that the construct contained the correct inserts with no PCR errors. The construct was designated pDM313.

### Example 16: Construction of Plasmid pQM43

Plasmid pQM43 was constructed for targeting a non-functional amdS fragment (designated “non-functional amdS fragment 3”) flanked at its 5′ by a FRT-F site to the T. reesei cbh1 gene locus. In the construct, the FRT-F site (49 bp) was added within the first intron of an approximately 1 kb non-functional amdS fragment as described below. An approximately 400 bp fragment containing the T. reesei cbh1 5′ flanking region and FRT-F fragment was amplified from T. reesei RutC30 genomic DNA using primers 1208187 and 1208194 shown below. The non-functional amdS fragment 3 flanked by a FRT-F site was amplified from pAllo1 (WO 04/111228) using primers 1208195 and 1208196 shown below.

Primer 1208187:

(SEQ ID NO: 59)

5′-GATTGAGTTGAAACTGCCTAAGATCTCG-3′
Primer 1208194:

(SEQ ID NO: 60)

5′-CTATACTTTCTAGAGAATAGGAACTCGGAATAGGAACTTCAAGGTGC
GCAGTCCGCGGTTGAC-3′
Primer 1208195:

(SEQ ID NO: 61)

5′-CTATTCCGAGTTCCTATTCTCTAGAAAGTATAGGAACTTCGGCGTTG
TTACATCTCCCCTGAG-3′
Primer 1208196:

(SEQ ID NO: 62)

5′-GCGTCAGGCTTTCGCCACGTCTACGCCAGGACCGAGCAAG-3′

The first PCR was composed of 100 ng of T. reesei RutC30 genomic DNA, 1 μl of 10 mM dNTPs, 1 μM primers, 1× PHUSION® High-Fidelity Reaction Buffer (Thermo Fisher Scientific, Inc.), and 1 unit of PHUSION® Hot Start High-Fidelity DNA Polymerase (Thermo Fisher Scientific, Inc.) in a final volume of 50 μl. The second PCR was composed of 100 ng of pAllo1, 1 μl of 10 mM dNTPs, 1 μM primers, 1× PHUSION® High-Fidelity Reaction Buffer, and 1 unit of PHUSION® Hot Start High-Fidelity DNA Polymerase in a final volume of 50 μl. Both reactions were performed in a thermocycler programmed for 1 cycle at 95° C. for 2 minutes; 35 cycles each at 95° C. for 15 seconds, 60° C. for 30 seconds, and 72° C. for 1 minute and 15 seconds; and 1 cycle at 72° C. for 10 minutes. The PCR products were separated by 0.7% agarose gel electrophoresis in TAE buffer where fragments of approximately 400 bp and 1 kb was excised from the gel and extracted using a Nucleospin® Extract II Kit (Example 14).

The third PCR was composed of 100 ng of each of the 400 bp and 1 kb purified PCR products, 1 μl of 10 mM dNTPs, 1× PHUSION® High-Fidelity Reaction Buffer, and 1 unit of PHUSION® Hot Start High-Fidelity DNA Polymerase in a final volume of 48 μl. The reaction was performed in a thermocycler programmed for 1 cycle at 95° C. for 2 minutes; and 5 cycles each at 95° C. for 15 seconds, 60° C. for 30 seconds, and 72° C. for 2 minutes. Primers 1208187 and 1208196 were then added at final concentrations of 1 μM and continued for 30 cycles each at 95° C. for 15 seconds, 60° C. for 30 seconds, and 72° C. for 2 minutes, and 1 cycle at 72° C. for 10 minutes. The PCR product was separated by 0.7% agarose gel electrophoresis in TAE buffer where a fragment of approximately 1.4 kb were excised from the gel and extracted using a Nucleospin® Extract II Kit as above.

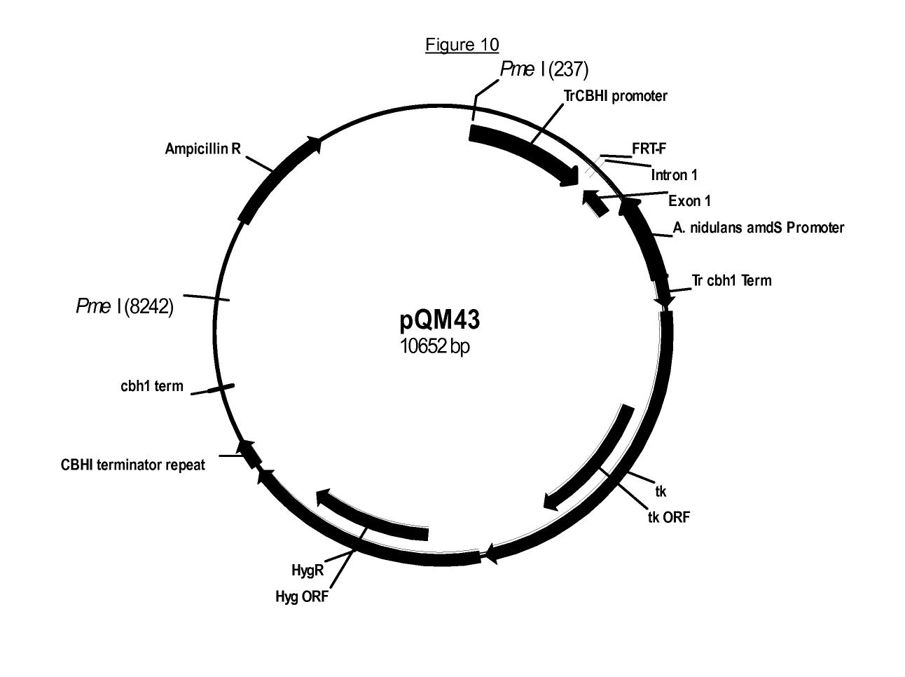

The 1.4 kb PCR product was inserted into an approximately 9.3 kb Bgl II/Pac I digested pJfyS139 (WO 2013/028927) using an IN-FUSION™ HD Cloning Kit (Clontech Laboratories, Inc.). The reaction was composed of 1× IN-FUSION™ HD Enzyme Premix (Clontech Laboratories, Inc.), 200 ng of Bgl II/Pac I digested pJfyS139, and 61 ng of the 1.4 kb PCR product in a 10 μl reaction volume. The reaction was incubated at 50° C. for 15 minutes. After the incubation period, a 1 μl aliquot was transformed into ONE SHOT® TOP10 Chemically Competent cells according to Example 13. Plasmid DNA was isolated from the transformants using a BIOROBOT® 9600. The insert was confirmed by DNA sequencing. One transformant was identified as containing the insert with no PCR errors and the plasmid was designated pQM43 (FIG. 10).

### Example 17: Protoplast Generation and Transformation of Trichoderma reesei Strain AgJg115-104-7B1 to Delete the T. reesei 42 kDa Aspartic Protease to Create T. reesei AgJg115-118-1H1

Protoplast preparation and transformation of Trichoderma reesei strain AgJg115-104-7B1 were performed according to Example 10.

Ninety-six μg of the transforming plasmid pAgJg118 (WO 2011/075677) was digested with Pme I and purified by 1% agarose gel electrophoresis in TAE buffer where a DNA band was excised from the gel and extracted using a QIAQUICK® Gel Extraction Kit (QIAGEN Inc.). Briefly 3 volumes of Kit-supplied Buffer QG were added to the gel slice and dissolved at 50° C. for approximately 10 minutes. The dissolved gel slice was transferred to a spin column and centrifuged at 13,000 rpm for 1 minute. The column was washed with 750 μl of Kit-supplied Buffer PE and then the centrifugation was repeated. DNA was eluted with 25 μl of Kit-supplied Buffer EB. Approximately 1 μg of the resulting purified DNA fragment was added to 100 μl of the protoplast solution for hygromycin selection transformation as described above. Seven transformants were sub-cultured onto new PDA plates to generate spores.

The transformants of T. reesei strain AgJg115-104-7B1 were screened by Fungal Spore PCR for the presence of the pAgJg118 deletion vector at the 42 kDa aspartic protease locus. A small amount of spores from each transformant was suspended in 20 μl of Dilution buffer (PHIRE® Plant Direct PCR Kit, Thermo Fisher Scientific Inc.). The spore suspensions were used as templates in the PCRs to screen for the aspartic protease gene deletion. Each reaction was composed of 0.5 μl of the spore suspension, 50 pmol of primer 069134 (shown below), 50 pmol of primer 067947 (shown below), 10 μl of 2× PHIRE® Plant PCR Buffer (PHIRE® Plant Direct PCR Kit), and 0.4 μl of PHIRE® Hot Start II DNA Polymerase (PHIRE® Plant Direct PCR Kit) in a 20 μl reaction. The reactions were performed in a thermocycler programmed for 1 cycle at 98° C. for 5 minutes; 40 cycles each at 98° C. for 5 seconds, 58° C. for 5 seconds, and 72° C. for 2 minutes and 20 seconds; 1 cycle at 72° C. for 2 minutes; and a 10° C. hold. Primer 069134 is located upstream of the 5′ flanking region and primer 067947 is located at the beginning of the E. coli hygromycin phosphotransferase (hpt) gene coding region. If the deletion vector integrates into the aspartic protease locus, the amplified PCR fragment will be 2.4 kb in length. One transformant designated T. reesei AgJg115-118-1 was identified as having the aspartic protease gene deleted.

Primer 069134 (forward):

(SEQ ID NO: 63)

5′-CGCAATCTATCGAATAGCAG-3′
Primer 067947 (reverse):

(SEQ ID NO: 64)

5′-CTACATCGAAGCTGAAAGCACGAGA-3′

The deletion construct pAgJg118 contains the E. coli hygromycin phosphotransferase (hpt) gene and the Herpes simplex virus thymidine kinase (tk) gene flanked by direct repeats. The direct repeats were inserted to facilitate the curing out of the hpt and tk selectable markers and generate a clean deletion of the 42 kDa aspartic protease.

Spores from T. reesei AgJg115-118-1 were spread onto Trichoderma Minimal medium plates containing 1 μM 5-fluoro-2′-deoxyuridine (FdU) and incubated at 28° C. Nine isolates were sub-cultured onto PDA plates and incubated at 28° C. The isolates were then screened for the absence of the hpt and tk markers by Fungal Spore PCR in a similar manner described above. The PCR screen was composed of 0.5 μl of the spore suspension, 50 pmol of primer 069134, 50 pmol of primer 1200593, 10 μl of 2× PHIRE® Plant PCR Buffer, and 0.4 μl of PHIRE® Hot Start II DNA Polymerase in a 20 μl reaction. The reaction was performed in a thermocycler programmed for 1 cycle at 98° C. for 5 minutes; 40 cycles each at 98° C. for 5 seconds, 58° C. for 5 seconds, and 72° C. for 1 minute and 45 seconds; 1 cycle at 72° C. for 1 minute; and a 10° C. hold. Primer 069134 is located upstream of the 5′ flanking region and primer 067947 is located at the downstream of the 3′ flanking region. If the aspartic protease coding sequence is deleted and the hpt and tk markers are looped out, the amplified PCR fragment will be 3.6 kb in length.

Genomic DNA of the T. reesei AgJg115-118-1 isolates was prepared as described in Example 11 and analyzed by Southern blot analysis as described in Example 12 to confirm the deletion of the 42 kDa aspartic protease. For Southern blot analysis, 2 μg of each genomic DNA was digested with 10 units of Nco I in a 30 μl reaction volume and subjected to 0.7% agarose gel electrophoresis in TAE buffer and transferred to a NYTRAN® SuperCharge membrane as described in Example 12.

The membrane was hybridized with a 500 bp digoxigenin-labeled T. reesei 42 kDa aspartic protease probe, which was synthesized by incorporation of digoxigenin-11-dUTP by PCR using the primers shown below.

Primer 069860 (sense):

(SEQ ID NO: 65)

5′-CTTCTATCTTGGGATGCTTCACGATACGTGA-3′
Primer 069861 (antisense):

(SEQ ID NO: 66)

5′-CGCGCCCTTGAATATCGGAGAAGGT-3′

The PCR was composed of 5 μl of 10× Taq Buffer (New England Biolabs, Inc.), 2.5 μl of PCR DIG Labeling Mix (Roche Applied Science Corp.), 5 ng of pAgJg118, 10 pmol of each primer, 2.5 μl of 10 mM dNTPs, 5 units of Taq DNA polymerase (New England Biolabs, Inc.), and 36.5 μl of water. The reaction was performed in a thermocycler programmed for 1 cycle at 95° C. for 2 minutes; 30 cycles each at 95° C. for 30 seconds, 56° C. for 30 seconds, and 72° C. for 40 seconds; 1 cycle at 72° C. for 15 minutes; and a 4° C. hold. The probe was purified by 1% agarose gel electrophoresis in TAE buffer, excised from the gel, and extracted using a QIAQUICK® Gel Extraction Kit as above.

Southern blot analysis identified primary transformant T. reesei AgJg115-118-1H1 as containing the replacement and being void of the hpt/tk markers.

### Example 18: Construction of T. reesei Strain QMJi057 for Targeting a Non-Functional amdS Fragment 3 Flanked by a FRT-F Site to the T. reesei cbh1 Locus

Trichoderma reesei AgJg115-118-1H1 (Example 17) was transformed with 1-5 μg of Pme I digested pQM43 to insert the non-functional amdS fragment 3 flanked at its 5′ by a FRT-F site at the cbh1 gene locus. Twenty-six transformants were obtained and each one was picked and transferred to a PDA plate and incubated for 7 days at 30° C. The transformants were cultured in 2 ml of CIM and incubated at 30° C. for 3 days with agitation at 250 rpm. Supernatant from each culture was subjected to SDS-PAGE using a CRITERION® 8-16% TGX Stain-Free gel (Bio-Rad Laboratories, Inc.) and PRECISION PLUS® Protein Unstained Standards (Bio-Rad Laboratories, Inc.). Since successful targeted integration of pQM43 at the cbh1 locus effectively disrupts the cbh1 gene, SDS-PAGE gels were visually analyzed for loss of the CBH1 protein from the proteome. T. reesei QMJi057-5 was identified as producing no CBHI protein and was selected for genomic DNA extraction and Southern blot analysis to confirm the integration of the FRT-F site containing the non-functional amdS fragment 3 at the T. reesei cbh1 gene locus. Genomic DNA was isolated from the transformants according to the procedure described in Example 11.

Genomic DNA was digested with Nhe I for Southern blot analysis according to Example 12 with a digoxigenin-labeled T. reesei cbh1 3′ probe synthesized by PCR using a PCR DIG Probe Synthesis Kit (Roche Applied Science Corp.) and the primers shown below.

Primer 0610249:

(SEQ ID NO: 67)

5′-GAGAACACAGTGAGACCATAGC-3′
Primer 0610250:

(SEQ ID NO: 68)

5′-TCTCAACCCAATCAGCAACATG-3′

The DIG Probe Synthesis PCR was composed of approximately 100 pg of a PCR fragment containing the T. reesei cbh1 3′ flanking region used to make pQM21 (WO 2013/028912) as template, 1 μM primers, 5 μl of PCR DIG Synthesis Mix (Roche Applied Science Corp.), 1×PCR buffer with MgCl₂ (Roche Applied Science Corp.), and 0.75 μl of Enzyme Mix (Roche Applied Science Corp.) in a final volume of 50 μl. The reaction was performed in a thermocycler programmed for 1 cycle at 95° C. for 2 minutes; 10 cycles each at 95° C. for 30 seconds, 60° C. for 30 seconds, and 72° C. for 40 seconds; 20 cycles each at 95° C. for 30 seconds, 60° C. for 30 seconds, and 72° C. for 40 seconds plus an additional 20 seconds for each successive cycle; and 1 cycle at 72° C. for 7 minutes. The PCR product was separated by 1% agarose gel electrophoresis in TAE buffer where a 720 bp band was excised from the gel and extracted using a Nucleospin® Extract II Kit (Example 14).

Transformant QMJi057-5 was confirmed by Southern blot analysis to contain the non-functional amdS fragment 3 flanked at its 5′ by a FRT-F site at the cbh1 locus, which resulted in a hybridized signal at approximately 7.1 kb recognized by the T. reesei cbh1 3′ probe.

### Example 19: Site Specific Integrations at T. reesei cbh1 Locus in Strain QMJi057-5

An approximately 3 kb DNA fragment containing the coding sequence of an A. niger mannosidase (SEQ ID NO: 69 for the DNA sequence and SEQ ID NO: 70 for the amino acid sequence) was amplified from A. niger Bo-1 derivative strain CKle47 with primer 0614762 and 0614763 and cloned into Nco I and Pac I digested pMJ09 (U.S. Pat. No. 8,318,458 B2; approximately 7.2 kb), resulting in plasmid pQM27 (SEQ ID NO: 71). The A. niger mannosidase expression cassette in pQM27 is followed by a functional amdS marker.

An approximately 6.7 kb DNA fragment containing an A. niger mannosidase expression cassette, a non-functional amdS fragment 4, and the FRT-F site was amplified from pQM27 with primer 1201956 and 1201606. The PCR was composed of 100 ng of pQM27, 200 μM dNTPs, 0.4 μM primers, 1× PHUSION® Reaction Buffer, and 1 units of PHUSION® High Fidelity DNA polymerase in a final volume of 50 μl. The reaction was performed in a thermocycler programmed for 1 cycle at 98° C. for 2 minutes; 35 cycles each at 98° C. for 15 seconds, 60° C. for 30 seconds, and 72° C. for 3.5 minutes; and 1 cycle at 72° C. for 10 minutes. The 6.7 kb PCR product was separated by 0.7% agarose gel electrophoresis in TAE, excised from the gel, and extracted using a Nucleospin® Extract II Kit (Example 14).

The purified 6.7 kb PCR product was used as template to amplify a FRT-F site containing fragment using primer 1201956 and 1201957. The resulting FRT-F site containing PCR fragment was separated by 0.7% agarose gel electrophoresis in TAE and then excised from the gel and extracted using a Nucleospin® Extract II Kit (Example 14).

Primer 1210956:

(SEQ ID NO: 72)

5′-TGATTACGAATTGTTTAAACGGATCCGAATGTAGGATTGTTATCC
G-3′
Primer 1201606:

(SEQ ID NO: 73)

5′-GAAGTTCCTATACTTTCTAGAGAATAGGAACTCGGAATAGGAACTTC
AACCTTATGGGACTATCAAGCTGAC-3′
Primer 1210957:

(SEQ ID NO: 74)

5′-GTTACATTGACGTACTTATAAGAAGTTCCTATACTTTCTAGAGAATA
GGA-3′

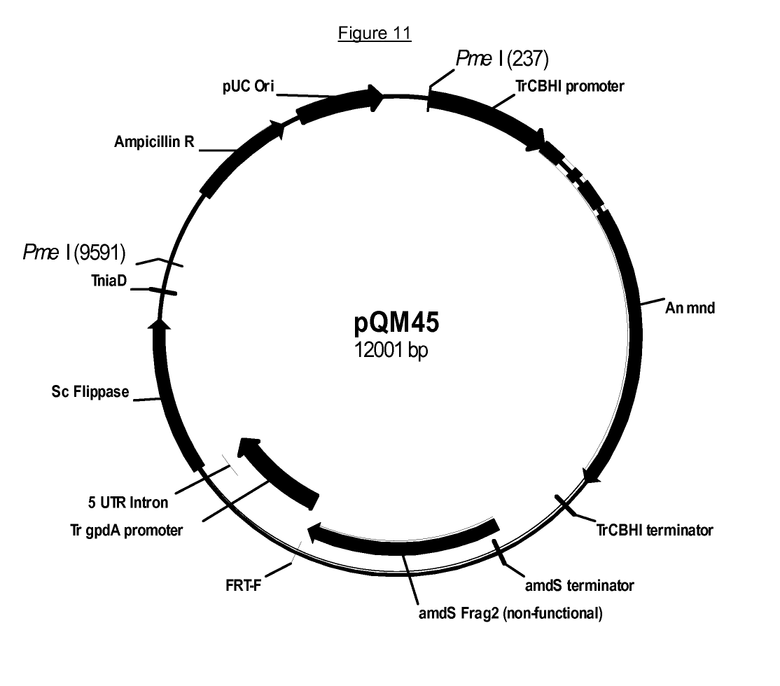

The purified FRT-F site containing PCR fragment was inserted into Xba I and Psi I digested pDM313 (approximately 5.4 kb) using an IN-FUSION™ HD Cloning Kit. The reaction was composed of 1× IN-FUSION™ HD Enzyme Premix, 323 ng of Xba I/Psi I digested pDM313, and 200 ng of the 6.7 kb FRT-F site containing PCR product in a 15 μl reaction volume. The reaction was incubated at 50° C. for 15 minutes. After the incubation period, a 2 μl aliquot was transformed into 50 μl of Stellar™ E. coli chemically competent cells (Clontech Laboratories, Inc.) by addition to a tube containing the competent cells and incubating the cells on ice for 30 minutes. The tube was incubated at 42° C. for 45 seconds after which 450 μl of SOC medium were added. The tube was then incubated at 37° C. with agitation at 250 rpm for 1 hour. Volumes of 50 μl and 150 μl were transferred to two 150 mm 2XYT plus ampicillin plates. The plates were incubated overnight at 37° C. Plasmid DNA was isolated using a BIOROBOT® 9600 and sequenced. One transformant was identified as containing the insert with no PCR errors and the plasmid was designated pQM45 (FIG. 11).

Plasmid pQM45 was digested with Pme I and used to test site specific integration at the T. reesei cbh1 locus in strain QMJi057-5. The Pme I digested pQM45 was transformed into protoplasts of T. reesei strain QMJi057-5 according to Example 10 and the transformation reactions were spread onto COVE plates and incubated at 30° C. for 7-10 days. All of the transformations resulted in visible transformants on COVE plates, suggesting that insertion of the FRT-F site into the first intron of the amdS gene allows recombination of a functional amdS marker and integration at the targeted site.

Genomic DNA was isolated from six transformants according to the procedure described in Example 11. Genomic DNA was digested with EcoR I for Southern blot analysis according to Example 12 with a 358 bp digoxigenin-labeled T. reesei cbh1 5′ probe (see below), which was synthesized by incorporation of digoxigenin-11-dUTP by PCR using a PCR DIG Probe Synthesis Kit.

Southern blot analysis of all six transformants resulted in a hybridized signal at approximately 4.2 kb recognized by the T. reesei cbh1 5′ probe, indicating correct integration at the cbh1 locus.

T. reesei cbh1 5′ probe sequence:

(SEQ ID NO: 75)

5′-TAGGGTCGGCAACGGCAAAAAAGCACGTGGCTCACCGAAAAGCAAGA
TGTTTGCGATCTAACATCCAGGAACCTGGATACATCCATCATCACGCACG
ACCACTTTGATCTGCTGTAAACTCGTATTCGCCCTAAACCGAAGTGCGTG
GTAAATCTACACGTGGGCCCCTTTCGGTATACTGCGTGTGTCTTCTCTAG
GTGCCATTCTTTTCCCTTCCTCTAGTGTTGAATTGTTTGTGTTGGAGTCC
GAGCTGTAACTACCTCTGAATCTCTGGAGAATGGTGGACTAACGACTACC
GTGCACCTGCATCATGTATATAATAGTGATCCTGAGAAGGGGGGTTTGGA
GCAATGTGGG-3′

### Example 20: Construction of Plasmid pQM37, pQM38 and pQM39 (Functional amdS Marker with FRT-F3 Sites in amdS Introns)

Plasmids pQM37, pQM38 and pQM39 were constructed to test the impact on amdS function after inserting a FRT-F3 fragment into one of the three introns of the amdS gene. The primers shown below were designed to introduce a FRT-F3 fragment into each of the three introns in the amdS gene in plasmid pAllo1 using a QUICKCHANGE® II XL Site-Directed Mutagenesis Kit. Primers 1205503 and 1205504 were used to construct plasmid pQM37, where the FRT-F3 site was inserted into intron 1 of the amdS gene. Primers 1205505 and 1205506 were used to construct plasmid pQM38, where the FRT-F3 site was inserted into intron 2 of the amdS gene. Primers 1205507 and 1205508 were used to construct pQM39, where the FRT-F3 site was inserted into intron 3 of the amdS gene.

Primer 1205503:

(SEQ ID NO: 76)

5′-GGGAGATGTAACAACGCCTTGAAGTTCCTATTCCGAGTTCCTATTCT
TCAAATAGTATAGGAACTTCAACCTTATGGGACTATCAAG-3′
Primer 1205504:

(SEQ ID NO: 77)

5′-CTTGATAGTCCCATAAGGTTGAAGTTCCTATACTATTTGAAGAATAG
GAACTCGGAATAGGAACTTCAAGGCGTTGTTACATCTCCCC-3′
Primer 1205505:

(SEQ ID NO: 78)

5′-GCCCCTAAGTCGTTAGATGTTTGAAGTTCCTATTCCGAGTTCCTATT
CTTCAAATAGTATAGGAACTTCACCCTTTTTGTCAGC-3′
Primer 1205506:

(SEQ ID NO: 79)

5′-GCTGACAAAAAGGGTGAAGTTCCTATACTATTTGAAGAATAGGAACT
CGGAATAGGAACTTCAAACATCTAACGACTTAGGGGC-3′
Primer 1205507:

(SEQ ID NO: 80)

5′-CTATACCAGGCCTCCACTTGAAGTTCCTATTCCGAGTTCCTATTCTT
CAAATAGTATAGGAACTTCATGTCCTCCTTTCTTGC-3′
Primer 1205508:

(SEQ ID NO: 81)

5′-GCAAGAAAGGAGGACATGAAGTTCCTATACTATTTGAAGAATAGGAA
CTCGGAATAGGAACTTCAAGTGGAGGCCTGGTATAG-3′

The PCRs were composed of 10 ng of pAllo1, 1 μl of 10 mM dNTPs, 1× Reaction Buffer, 125 ng of each primer, 1 μl of QUIKSOLUTION® reagent, and 2.5 unit of PfuUltra™ HF DNA Polymerase (Agilent Technologies) in a final volume of 50 μl. The reactions were performed in a thermocycler programmed for 1 cycle at 95° C. for 1 minutes; and 18 cycles each at 95° C. for 50 seconds, 60° C. for 50 seconds, and 68° C. for 7 minutes; and 1 cycle at 68° C. for 7 minutes. Ten units of Dpn I were added to each reaction and incubated at 37° C. for one hour. Two μl of each Dpn I treated reaction were transformed into XL10-Gold Ultracompetent E. coli cells. The transformation reactions were spread onto 2XYT plus ampicillin plates. About 4-6 colonies were picked for each transformation and cultured in 3 ml of LB plus ampicillin medium at 37° C. for 15-17 hours with agitation at 250 rpm. Plasmid DNA was extracted from each colony using a QIAprep® Spin Miniprep Kit and submitted for DNA sequencing. One transformant containing the FRT-F3 site inserted into intron 1 of the amdS gene was designated pQM37. One transformant containing the FRT-F3 site inserted into intron 2 of the amdS gene was designated pQM38. One transformant containing the FRT-F3 site inserted into intron 3 of the amdS gene was designated pQM39.

About 30-40 μg of each of pAllo1, pQM37, pQM38, and pQM39 were digested with Eco RI and Pac I. The digested DNAs were purified using a Nucleospin® Extract II Kit (Example 14). The purified linear DNA fragments of pAllo1, pQM37, pQM38, and pQM39 were each transformed into T. reesei RutC30 protoplasts according to Example 10. The transformation reactions were spread onto COVE plates and incubated at 28° C. for 7-10 days. All of the transformations resulted in visible transformants on COVE plates, indicating that amdS can be used as a functional selection marker with a FRT-F3 site inserted in any one of the three amdS introns.

1. A method for integrating a polynucleotide library of interest in the chromosome of a filamentous fungal host cell using a site-specific recombinase, said method comprising the steps of:
a) providing a filamentous fungal host cell comprising in its chromosome in the following order:
i) a first recognition sequence of the recombinase or a region that is 5′ or 3′ of an integration site;
ii) a first selection marker;
iii) a second recognition sequence of the recombinase; and optionally
iv) a non-functional partial second selection marker;
b) transforming said host cell with a nucleic acid construct comprising in the following order:
i) the first recognition sequence of the recombinase or the region that is 5′ or 3′ of the integration site;
ii) a polynucleotide library of interest;
iii) a second selection marker or a non-functional partial second selection marker if the corresponding but optional non-functional second selection marker of step (a)(iv) is comprised in the host cell chromosome; and
iv) the second recognition sequence of the recombinase;
c) expressing a gene encoding the site-specific recombinase in said host cell; and
d) selecting a transformed host cell which expresses the second selection marker and not the first selection marker,
wherein the polynucleotide library of interest is site-specifically integrated in the correct orientation in the chromosome of the host cell by the recombinase, whereby the first selectable marker is excised from the chromosome and whereby any non-functional partial second selectable markers are recombined to form a functional second selection marker in the chromosome.

2. The method of claim 1, wherein the polynucleotide library comprises polynucleotides encoding variants of a polypeptide of interest; preferably the polypeptide of interest is an enzyme; more preferably the polypeptide of interest is a hydrolase, isomerase, ligase, lyase, oxidoreductase, or transferase, e.g., an aminopeptidase, amylase, carbohydrase, carboxypeptidase, catalase, cellobiohydrolase, cellulase, chitinase, cutinase, cyclodextrin glycosyltransferase, deoxyribonuclease, endoglucanase, esterase, alpha-galactosidase, beta-galactosidase, glucoamylase, alpha-glucosidase, beta-glucosidase, invertase, laccase, lipase, mannosidase, mutanase, oxidase, pectinolytic enzyme, peroxidase, phytase, polyphenoloxidase, proteolytic enzyme, ribonuclease, transglutaminase, xylanase, or beta-xylosidase.

3. (canceled)

4. The method of claim 1, wherein the site specific recombinase is the Saccharomyces cerevisiae 2 μm flippase FLP, the phage TP901-1 integrase, the bacteriophage P1 CRE integrase, the bacterial XerC recombinase, the bacterial XerD recombinase, the lambda phage integrase or the HP1 integrase; preferably the site specific recombinase is the Saccharomyces cerevisiae 2 μm flippase FLP.

5. The method of claim 1, wherein the first and second recognition sequences of the recombinase are identical or different; preferably the first and second recognition sequences of the recombinase are different in order to effect a directional integration of the polynucleotide library of interest in the host cell chromosome.

6. The method of claim 1, wherein the first and second recognition sequences of the recombinase are different recognition sequences of the Saccharomyces cerevisiae 2 μm flippase FLP in order to effect a directional integration of the polynucleotide library of interest in the host cell chromosome; preferably the first and second recognition sequences of the recombinase are FRT-F (SEQ ID NO:27) and FRT-F3 (SEQ ID NO:28), respectively, or vice versa.

7. The method of claim 1, wherein one non-functional partial second selection marker is comprised in the host cell of claim 1 step (a) and another non-functional partial second selection marker is comprised in the nucleic acid construct of claim 1 step (b) and wherein the partial second selection markers are recombined to form a functional second selection marker when the polynucleotide library of interest is site-specifically integrated in the correct orientation in the chromosome of the host cell by the recombinase via its recognition sequences.

8. The method of claim 7, wherein one non-functional partial second selection marker comprises a promoter and, optionally, one or more intact 5′ exon of a polynucleotide encoding a selection marker, and, wherein the other non-functional partial second selection marker comprises the remaining coding sequence of the selection marker.

9. A polynucleotide library expression system comprising:
a) a filamentous fungal host cell comprising in its chromosome in the following order:
i) a first recognition sequence of a site-specific recombinase or a region that is 5′ or 3′ of an integration site;
ii) a first selection marker;
iii) a second recognition sequence of the recombinase; and optionally
iv) a non-functional partial second selection marker; and
b) a nucleic acid construct comprising in the following elements in order:
i) the first recognition sequence of the recombinase or the region that is 5′ or 3′ of the integration site;
ii) a polynucleotide library of interest;
iii) a second selection marker or a non-functional partial second selection marker if the optional non-functional second selection marker of step (a)(iv) is comprised in the host cell chromosome; and
iv) the second recognition sequence of the recombinase, and
c) a gene encoding the site-specific recombinase comprised in the filamentous fungal host cell chromosome or in the nucleic acid construct outside of the elements listed in step (b);
wherein, when the host cell is transformed with the nucleic acid construct, the polynucleotide library of interest is site-specifically integrated in the correct orientation in the chromosome of the host cell by the recombinase, whereby the first selectable marker is excised from the chromosome, and whereby the second selection marker is also integrated and expressed or any non-functional partial second selectable markers are recombined to form a functional second selection marker in the chromosome which is expressed.

10. The polynucleotide library expression system of claim 9, wherein the polynucleotide library comprises polynucleotides encoding variants of a polypeptide of interest; preferably the polypeptide of interest is an enzyme; more preferably the polypeptide of interest is a hydrolase, isomerase, ligase, lyase, oxidoreductase, or transferase, e.g., an aminopeptidase, amylase, carbohydrase, carboxypeptidase, catalase, cellobiohydrolase, cellulase, chitinase, cutinase, cyclodextrin glycosyltransferase, deoxyribonuclease, endoglucanase, esterase, alpha-galactosidase, beta-galactosidase, glucoamylase, alpha-glucosidase, beta-glucosidase, invertase, laccase, lipase, mannosidase, mutanase, oxidase, pectinolytic enzyme, peroxidase, phytase, polyphenoloxidase, proteolytic enzyme, ribonuclease, transglutaminase, xylanase, or beta-xylosidase.

11. (canceled)

12. The polynucleotide library expression system of claim 9, wherein the site specific recombinase is the Saccharomyces cerevisiae 2 μm flippase FLP, the phage TP901-1 integrase, the bacteriophage P1 CRE integrase, the bacterial XerC recombinase, the bacterial XerD recombinase, the lambda phage integrase or the HP1 integrase; preferably the site specific recombinase is the Saccharomyces cerevisiae 2 μm flippase FLP.

13. The polynucleotide library expression system of claim 9, wherein the first and second recognition sequences of the recombinase are identical or different; preferably the first and second recognition sequences of the recombinase are different in order to effect a directional integration of the polynucleotide library of interest in the host cell chromosome.

14. The polynucleotide library expression system of claim 9, wherein the first and second recognition sequences of the recombinase are different recognition sequences of the Saccharomyces cerevisiae 2 μm flippase FLP in order to effect a directional integration of the polynucleotide library of interest in the host cell chromosome; preferably the first and second recognition sequences of the recombinase are FRT-F (SEQ ID NO:27) and FRT-F3 (SEQ ID NO:28), respectively, or vice versa.

15. The polynucleotide library expression system of claim 9, wherein one non-functional partial second selection marker is comprised in the host cell and another non-functional partial second selection marker is comprised in the nucleic acid construct, wherein the partial second selection markers are recombined to form a functional second selection marker when the polynucleotide library of interest is site-specifically integrated in the correct orientation in the chromosome of the host cell by the recombinase via its recognition sequences.

16. The polynucleotide library expression system of claim 15, wherein one non-functional partial second selection marker comprises a promoter and, optionally, one or more intact 5′ exon of a polynucleotide encoding a selection marker, and, wherein the other non-functional partial second selection marker comprises the remaining coding sequence of the selection marker.

17. A filamentous fungal host cell comprising in its chromosome in the following order:
i) a first recognition sequence of a site-specific recombinase or a region that is 5′ or 3′ of an integration site;
ii) a polynucleotide library of interest; and either
iii) a selection marker and a second recognition sequence of the recombinase; or
iv) a first partial selection marker, a second recognition sequence of the recombinase and a second partial selection marker,
wherein the host cell expresses the polynucleotide library and the selection marker.

18. The filamentous fungal host cell of claim 17, wherein the polynucleotide library comprises polynucleotides encoding variants of a polypeptide of interest; preferably the polypeptide of interest is an enzyme; more preferably the polypeptide of interest is a hydrolase, isomerase, ligase, lyase, oxidoreductase, or transferase, e.g., an aminopeptidase, amylase, carbohydrase, carboxypeptidase, catalase, cellobiohydrolase, cellulase, chitinase, cutinase, cyclodextrin glycosyltransferase, deoxyribonuclease, endoglucanase, esterase, alpha-galactosidase, beta-galactosidase, glucoamylase, alpha-glucosidase, beta-glucosidase, invertase, laccase, lipase, mannosidase, mutanase, oxidase, pectinolytic enzyme, peroxidase, phytase, polyphenoloxidase, proteolytic enzyme, ribonuclease, transglutaminase, xylanase, or beta-xylosidase.

19. (canceled)

20. The filamentous fungal host cell of claim 17, wherein the site specific recombinase is the Saccharomyces cerevisiae 2 μm flippase FLP, the phage TP901-1 integrase, the bacteriophage P1 CRE integrase, the bacterial XerC recombinase, the bacterial XerD recombinase, the lambda phage integrase or the HP1 integrase; preferably the site specific recombinase is the Saccharomyces cerevisiae 2 μm flippase FLP.

21. The filamentous fungal host cell of claim 17, wherein the first and second recognition sequences of the recombinase are identical or different; preferably the first and second recognition sequences of the recombinase are different.

22. The filamentous fungal host cell of claim 17, wherein the first and second recognition sequences of the recombinase are different recognition sequences of the Saccharomyces cerevisiae 2 μm flippase FLP; preferably the first and second recognition sequences of the recombinase are FRT-F (SEQ ID NO:27) and FRT-F3 (SEQ ID NO:28), respectively, or vice versa.

23. The filamentous fungal host cell of claim 22, wherein the second recognition sequence of the recombinase is located in an intron separating the first partial selection marker from the second partial selection marker.

24. A method of producing a polypeptide of interest, comprising the steps of:
a) cultivating a filamentous fungal host cell of claim 17 under conditions conducive to produce the polypeptide encoded by the polynucleotide library, and; optionally
b) recovering the polypeptide.


SEQUENCE LISTING (81 sequences)

SEQ ID NO: 1 - DNA, 16935 bp, artificial sequence
  Plasmid p002
  misc_feature (11869)..(11869): n is a, c, g, or t
ttaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggagctccaccgcggcaacaggcagaatatcttccgaattcaatcgactgcgcgatgcaagttggctagcaacggcgtacaccttgggattatgcgctgctcaaccgatggtcagctatcaaacaaaatttgggaagatcgggctatactgacggtgacattatagtacggcaagctgagtgacatctacggtcgcaagccactgcttctttgggcatatgttttctttggcgtgggatgcattatcaggtagatactccctttttcttatacgctggtttgctggttcgtgctgacagctgtttccctagcggtattggtcgagacatggcgactgtcatattggggcgtgcaatcagcggaattgggggtgctggaacaatggcgatgggctctatcattatcacaggtaggctagcagcttatcaggttgaaagaactgtcactgaacataggcagatattgttcctcgtcgagatgttgcccattggcgggcgtacatcaatatcgcgatgactctgggtcgtagcgcaggaggcccaatcggcggatggctaaccgatacaatcggatggagatggtatgctttgcgcctttgtgaccgcttctctcactaaattgtggccaaggtcgtttattatccaaggccccttagccgctgtggcagctctgttggtgatatggaagctcaaactcgccaatccagtcactgagaagagcatccgccgtgtcgactttctcggaacattcctcctggccgtcggtattgttacaatcaccgttatcatggaccaagcagggcagtccttcgcatgggcatcattgtcaacagcaatccttgcaactctcagtctatcagcattcgtcgccttcgtccttgttgaactctacgtagcccctgaaccgattttcgaacttcgcatgttgcggaagccgaatgtgacgcccagttacctgatcggatcgctgcagatcaccgcccaagttggaatgatgttctccgtgccgttatattttcaggtgacatcgaaagcctctgccaccgtagctggagggcatctggttcctgcagtgatcggaaacacgcttggcggcttaatcgcgggagcctttatccgtcgcaccggccaattcaaggtcctcttgatccttgccggtctcgttgcgtccgtcgcctatctactcctcatccttcgctggaacggtcatactggattctgggagtccttgtacattattcccggtggtatgggtactggtttctgctctgcagctgcttttgtcagtatgacggcgtttttgatgccgcaggaagtggccatggcaacaggaggttacttcctattattcagcttcgccatgacggccggtgtcactgtcactaacagtctgctggggacggttttcaagcgccagatggaacagcacctgacgggtccaggagccaagaaggttggtatccccgcaccttttctgcgtcacttactaacgagtatatgaagatcatcgagcgcgcgctgtccgacaccagctatatcaacggtttgcagggtcatgtccgggatgtagtggtaaaaggatatgtgactggtctccgctacacttactgtaagtcgtttgaatcatgcatccaccgtccaccttattaacttggtgccagtattttccctcattctttcgctccttggatcggtcctcgcttggactgtacgaaaacaccaactatgaggaaccagcacggcagctgatagtatccgaaagctgcaaattgcttcatcgaggctggcattcgatagaagaaagaactatagacaactagtcttacaatatgacaattctctttgattaataaatgaaaataacacttgtgtcagcctaatagccgagtggcgggcatctctggcggcctcccgagcagcgtggatctggaagtgcgttgatcattattccccgaaaatgtagtacccagtaagtggtctagcggtggctatggtaggacatctatgcctaagctggagttctcattgaacgtgtaccggccgattgccctaaactctgattgagagccggaaacctcatctacctgatgctcaggggccatccaatagcttccgatagcattacagacagatggactcgtcttggcccacgggtctagaacagtcgccggaactgcctctatttgaaacggagctgaaccatgatacttaagcgtgccaagcggcgccgtttcccactggaacaaggagcaatagaattctgcagagattcttcattcaggctattcagcaattcggtttgtggagcggatcggggtccactgggtttagtctggggtttttctttgcccgcatgggctctagcacatgcacagcttgcagttgctgctacgctatctgggaaaacgaatggctattcaggagtttataaccaaaagagccggaaacaggctgattgccctctcacggggagacgttgtacttctgatccagaggctattaaccggacactacctataaaggaggtagcattcctttctgtccggctcccagattccaacaacccaactgacaggggatccaccatgccccagttcgatatcctctgcaagaccccccccaaggtcctcgtccgccagttcgtcgagcgcttcgagcgcccctccggcgagaagatcgccctctgcgccgccgagctcacctacctctgctggatgatcacccataacggcaccgccatcaagcgcgccaccttcatgtcctacaacaccatcatctccaactccctctccttcgatatcgtcaacaagtccctccagttcaagtacaagacccagaaggccaccatcctggaggcctccctcaagaagctcatccccgcctgggagttcaccatcatcccctactacggccagaagcatcagtccgatatcaccgatatcgtctcctccctccagctccagttcgagtcctccgaggaggccgataagggcaactcccattccaagaagatgctcaaggccctcctctccgagggcgagtccatctgggagatcaccgagaagatcctcaactccttcgagtacacctcccgcttcaccaagaccaagaccctctaccagttcctcttcctcgccaccttcatcaactgcggccgcttctccgatatcaagaacgtcgatcccaagtccttcaagctcgtccagaacaagtacctcggcgtcatcatccagtgcctcgtcaccgagaccaagacctccgtctcccgccatatctacttcttctccgcccgcggccgcatcgatcccctcgtctacctcgatgagttcctccgcaactccgagcccgtcctcaagcgcgtcaaccgcaccggcaactcctcctccaacaagcaggagtaccagctcctcaaggataacctcgtccgctcctacaacaaggccctcaagaagaacgccccctactccatcttcgccatcaagaacggccccaagtcccatatcggccgccatctcatgacctccttcctctccatgaagggcctcaccgagctcaccaacgtcgtcggcaactggtccgataagcgcgcctccgccgtcgcccgcaccacctacacccatcagatcaccgccatccccgatcattacttcgcactagtctcccgctactacgcctacgatcccatctccaaggagatgatcgccctcaaggatgagaccaaccccatcgaggagtggcagcatatcgagcagctcaagggctccgccgagggctccatccgctaccccgcctggaacggcatcatctcccaggaggtcctcgattacctctcctcctacatcaaccgccgcatctgagtcgagattatccaagggaatgacttaatgagtatgtaagacatgggtcataacggcgttcgaaacatatacagggttatgtttgggaatagcacacgaataataacgttaataggtaccaaagtccttgatacattagcacggtagaaaaagaataatacaacgagctgggaatattctttaatataaaactccaagaagagctggtgcggtggagcttgttttcgactctcagtaatatttcctcatatccaagcgcgctaggaggtggtcgaatacacatgtaggcgcttctctggatgcaaaagtcgtgccggacctgccgaaagactttgaagatgcgttcacgccatctaagttgcgtagataattcacaaaaagggatgtttgtttccggaatgtagcaaagagctgataggcaatagcctcactttcgtggcgcacgccgctcgttccatccatcctcgacaatggagcaaatgtcaaaatcgtaccgaaaatactttgctagcccgttaaattgccgtcgtcagccgttaaattaccgattaatcccgataaatttccgagatctccgttaaattgccgttcgcagccgttaaattaccggggacgaccgataaatttccgcgatgaattcatggtgttttgatcattttaaatttttatatggcgggtggtgggcaactcgcttgcgcgggcaactcgcttaccgattacgttagggctgatatttacgtaaaaatcgtcaagggatgcaagaccaaaccgttaaatttccggagtcaacagcatccaagcccaagtccttcacggagaaaccccagcgtccacatcacgagcgaaggaccacctctaggcatcggacgcaccatccaattagaagcagcaaagcgaaacagcccaagaaaaaggtcggcccgtcggccttttctgcaacgctgatcacgggcagcgatccaaccaacaccctccagagtgactaggggcggaaatttatcgggattaatttccactcaaccacaaatcacagtcgtccccggtaatttaacggctgcagacggcaatttaacggcttctgcgaatcgcttggattccccgcccctggccgtagagcttaaagtatgtcccttgtcgatgcgatgtatcacaacatataaatactggcaagggatgccatgcttggagcaacaatctcagaacaccaatatcaattcatctcgagtctgaactcagacaacacaaattcttcaaaattgaggatttagtcttgatcttgaagttcctattccgagttcctattctctagaaagtataggaacttcttaattaacctagccgttattactctaccgcaaggcaacaaccagctcacccctgaggcacgggtaccatgggttgagtggtatggggccatccagagtcacctgtggcagcatgagactgcactcgaagcagccatcaacccagccaatattctgggctttccatccttagatcacatttgagatataacccatttggtgagagacacttgtgccgttatacgtgtctagactggaaacgcaaccctgaagggattcttcctttgagagatggaagcgtgtcatatctcttcggttctacggcaggtttttttctgctctttcgtagcatggcatggtcacttcagcgcttatttacagttgctggtattgatttcttgtgcaaattgctatctgacacttattaggtcgagctattcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatggcctccgcgaccggctgcagaacagcgggcagttcggtttcaggcaggtcttgcaacgtgacaccctgtgcacggcgggagatgcaataggtcaggctctcgctgaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggcgacattgctgaggtgtaggatcgatccgtttaaactctgtgttagcttatagtcaggatgttggctcgacgagtgtaaactgggagttggcatgagggttatgtaggcttctttagccccgcatccccctcattctcctcattgatcccgggggagcggatggtgttgataagagactaattatagggtttagctggtgcctagctggtgattggctggcttcgccgaattttacgggccaagggaagctgcagaaccgcggcactggtaaacggtaattaagctatcagccccatgctaacgagtttaaattacgtgtattgctgataaacaccaacagagctttactgaaagatgggagtcacggtgtggcttccccactgcgattattgcacaagcagcgagggcgaacttgactgtcgtcgctgagcagcctgcagtcaaacatacatatatatcaaccgcgaagacgtctggccttgtagaacacgacgctccctagcaacacctgccgtgtcagcctctacggttgttacttgcattcaggatgctctccagcgggcgagctattcaaaatattcaaagcaggtatctcgtattgccaggattcagctgaagcaacaggtgccaaggaaatctgcgtcggttctcatctgggcttgctcggtcctggcgtagactagtgcatgcttgaagttcctattccgagttcctattcttcaaatagtataggaacttcaaccatgcctcaatcctgggaagaactggccgctgataagcgcgcccgcctcgcaaaaaccatccctgatgaatggaaagtccagacgctgcctgcggaagacagcgttattgatttcccaaagaaatcgggtatcctttcagaggccgaactgaagatcacagaggcctccgctgcagatcttgtgtccaagctggcggccggagagttgacctcggcggaagttacgctagcattctgtaaacgggcagtaatcgcccagcagttagtagggtcccctctacctctcagggagatgtaacaacgccaccttatgggactatcaagctgacgctggcttctgtgcagacaaactgcgcccacgagttcttccctgacgccgctctcgcgcaggcaagggaactcgatgaatactacgcaaagcacaagagacccgttggtccactccatggcctccccatctctctcaaagaccagcttcgagtcaaggtacaccgttgcccctaagtcgttagatgtccctttttgtcagctaacatatgccaccagggctacgaaacatcaatgggctacatctcatggctaaacaagtacgacgaaggggactcggttctgacaaccatgctccgcaaagccggtgccgtcttctacgtcaagacctctgtcccgcagaccctgatggtctgcgagacagtcaacaacatcatcgggcgcaccgtcaacccacgcaacaagaactggtcgtgcggcggcagttctggtggtgagggtgcgatcgttgggattcgtggtggcgtcatcggtgtaggaacggatatcggtggctcgattcgagtgccggccgcgttcaacttcctgtacggtctaaggccgagtcatgggcggctgccgtatgcaaagatggcgaacagcatggagggtcaggagacggtgcacagcgttgtcgggccgattacgcactctgttgagggtgagtccttcgcctcttccttcttttcctgctctataccaggcctccactgtcctcctttcttgctttttatactatatacgagaccggcagtcactgatgaagtatgttagacctccgcctcttcaccaaatccgtcctcggtcaggagccatggaaatacgactccaaggtcatccccatgccctggcgccagtccgagtcggacattattgcctccaagatcaagaacggcgggctcaatatcggctactacaacttcgacggcaatgtccttccacaccctcctatcctgcgcggcgtggaaaccaccgtcgccgcactcgccaaagccggtcacaccgtgaccccgtggacgccatacaagcacgatttcggccacgatctcatctcccatatctacgcggctgacggcagcgccgacgtaatgcgcgatatcagtgcatccggcgagccggcgattccaaatatcaaagacctactgaacccgaacatcaaagctgttaacatgaacgagctctgggacacgcatctccagaagtggaattaccagatggagtaccttgagaaatggcgggaggctgaagaaaaggccgggaaggaactggacgccatcatcgcgccgattacgcctaccgctgcggtacggcatgaccagttccggtactatgggtatgcctctgtgatcaacctgctggatttcacgagcgtggttgttccggttacctttgcggataagaacatcgataagaagaatgagagtttcaaggcggttagtgagcttgatgccctcgtgcaggaagagtatgatccggaggcgtaccatggggcaccggttgcagtgcaggttatcggacggagactcagtgaagagaggacgttggcgattgcagaggaagtggggaagttgctgggaaatgtggtgactccataggtaagctccgtggcgaaagcctgacgcaccggtagattcttggtgagcccgtatcatgacggcggcgggagctacatggccccgggtgatttattttttttgtatctacttctgacccttttcaaatatacggtcaactcatctttcactggagatgcggcctgcttggtattgcgatgttgtcagcttggcaaattgtggctttcgaaaacacaaaacgattccttagtagccatgcattttaagataacggaatagaagaaagaggaaattaaaaaaaaaaaaaaaacaaacatcccgttcataacccgtagaatcgccgctcttcgtgtatcccagtaccacggcaaaggtatttcatgatcgttcaatgttgatattgttcccgccagtatggctccacccccatctccgcgaatctcctcttctcgaacgcggtagtggcgcgccaattggtaatgacccatagggagacaaacagcataatagcaacagtggaaattagtggcgcaataattgagaacacagtgagaccatagctggcggcctggaaagcactgttggagaccaacttgtccgttgcgaggccaacttgcattgtctagagaatgcaatcataacagaaagtacagccagcgctgtgtcataaagaagtccagttgggaaacgaaagactagaatcaaactaaaagtaatccggccgatatggcttcacgtgcgaagtctcgccttgaggggacattgtccttgcaggtgattgaccattgcgttcatatggcgcgatgtttggtagtgtgggtgtagccggtgacctcacggaaggactaaaggccacatacccttctgagtgcctcttctcttcgtggtcggaactctcgaatgggtttttgacagttgcactcgtttggttgtggtcatttgaaggtctgcgttcggtcttctgttcgcgcaggcgagctgactgagggattgaaagctgcatagccatcgttggcatgcgttaattcgccaaagctcagcggcgaaacaggcctgacctctaatccatgcatctgctctgcactcgattgttcgtggtgtccttgcgaagaaagagaagccttggactcggatgactttctggacgaggtggtaggatcatcatgattgtaatgagactgtagcacatcatgcgaatcattcgacacacggtgtctgcccaagttgacgtcagcatcggtatgcatttcggtatggtcctcatggttctcagcatgtccctccagaggggactcatttccagcggaaggattataagcaacataattgtcatgtggctgcgacctttcgtgagactccgagtttgatctcactgtggactcatgggcgatatgcggctcatcatgatcttcgaatggagagaaatggttgaagtcggaggacacgggtgatttagcagcagggttgaatgcaacatagccagtctcgcgctcttcatgtgagctatatgagtcatgtggcctgtcatggtccagaggctccggatgctcatggctagattcatcgtgtgccgaaatcgcgtcactagcaaagggcgaggttgacacattggctgcaggactgaacgccacataaccactctcaggctcttcatgtgagttataggagctgtgcggcatatcatagtcctgaggttcacgatgctcatggctggattcatcgtgtgccgaaatagcgtgactagcaaaaggcgagggcgaagcattggttgccggactgaacgccacatagccgtccccattggctgaactgactggtgacaacgtcctacccatggcgtcggccgggccagcggcttggtgagagtgaagaccattagaagtagctggactgaacgatcgcagcgggtattcgttttcttgagctggataaggggctgcgccatgctggctgaaaggtgagaatgttcggggtgctgctctatcaccagggaaggcagacgctggagtcaaagaacgagtgttggatcaattgccggactgtatgaacggaaaggagtgctgattgtttaaatggcccgtagccttcgctaggacctcgtgattcggggaccgttggcccatacccaggagctggtgtaaaattggaacgcgacacgggtgtttggttgcgcagaatttgcggtgccggcgaggcgtgatcaatctggctgtaacctgggcctggggtgtagtttgagacaggtgtttgtgttcgtggcatttgtggcgctggcgacgctctgtcagtcggcccatatccaggcgccgaaggtgtgggcgtaaacccttgccgtgatttaattaagaattctcctgtaggcttgagagttcaaggaagaaacagtgcaattatctttgcgaacccaggngctggtgacggaattttcatagtcaagctatcagagtaaagaagaggagcatgtcaaagtacaattagagacaaatatatagtcgcgtggagccaagagcggattcctcagtctcgtaggtctcttgacgaccgttgatctgcttgatctcgtctcccgaaaatgaaaatagactctgctaagctattcttctgcttcgccggagcctgaagggcgtactagggttgcgaggtccaatgcattaatgcattgcagatgagctgtatctggaagaggtaaacccgaaacgcgttttattcttgttgacatggagctattaaatcactagaaggcactctttgctgcttggacaaatgaacgtatcttatcgagatcctgaacaccatttgtctcaactccggagctgacatcgacaccaacgatcttatatccagattcgtcaagctgtttgatgatttcagtaacgttaagtggatcgatccggatagcgcgggttccttccggtattgtctccttccgtgtttcagttagcctcccccatctcccgggcaaacgtgcgcgccaggtcgcatatcgtcggtatggagccgggggtggtgacgtgggtctggaccatcccggaggtaagttgcagcagggcgtcccggcagccggcgggcgattggtcgtaatccaggataaagacgtgcatggaacggaggcgtttggccaagacgtccaaggcccaggcaaacacgttatacaggtcgccgttgggggccagcaactcggggccccgaaacagggtaaataacgtgtccccgatatggggtcgtgggcccgcgttgctctggggctcggcaccctggggcggcacggccgtccccgaaagctgtccccagtcctcccgccacgacccgccgcactgcagataccgcaccgtattggcaagtagcccgtaaacgcggcgaatcgcagccagcatagccaggtccagccgctcgccggggcgctggcgtttggccaggcggtcgatgtgtctgtcctccggaagggccccaagcacgatgttggtgccgggcaaggtcggcgggatgagggccacgaacgccagcacggcctggggggtcatgctgcccataaggtaccgcgcggccgggtagcacaggagggcggcgatgggatggcggtcgaagatgagggtgagggccgggggcggggcatgtgagctcccagcctcccccccgatatgaggagccagaacggcgtcggtcacggcataaggcatgcccattgttatctgggcgcttgtcattaccaccgccgcgtccccggccgatatctcaccctggtcgaggcggtgttgtgtggtgtagatgttcgcgattgtctcggaagcccccagcacccgccagtaagtcatcggctcgggtacgtagacgatatcgtcgcgcgaacccagggccaccagcagttgcgtggtggtggttttccccatcccgtggggaccgtctatataaacccgcagtagcgtgggcattttctgctccgggcggacttccgtggcttcttgctgccggcgagggcgcaacgccgtacgtcggttgctatggccgcgagaacgcgcagcctggtcgaacgcagacgcgtgttgatggccggggtacgaagccatacgcgcttctacaaggcgctggccgaagaggtgcgggagtttcacgccaccaagatcgatctacagctggtggggagagcaggaaaatatggcaacaaatgttggactgacgcaacgaccttgtcaaccccgccgacacaccgggcggacagacggggcaaagctgcctaccagggactgagggacctcagcaggtcgagtgcagagcaccggatgggtcgactgccagcttgtgttcccggtctgcgccgctggccagctcctgagcggcctttccggtttcatacaccgggcaaagcaggagaggcacgatatttggacgccctacagatgccggatgggccaattagggagcttacgcgccgggtactcgctctacctacttcggagaaggtactatctcgtgaatcttttaccagatcggaagcaattggacttctgtacctaggttaatggcatgctatttcgccgacggctatacacccctggcttcacattctccttcgcttactgccggtgattcgatgaagctccatattctccgatgatgcaatagattcttggtcaacgaggggcacaccagcctttccacttcggggcggaggggcggccggtcccggattaataatcatccactgcacctcagagccgccagagctgtctggcgcagtggccgttattactcagcccttctctctgcgtccgtccgtctctccgcatgccagaaagagtcaccggtcactgtacagagctcaagcttcgattaactcgagggggggcccggtacccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattca

SEQ ID NO: 2 - DNA, 49 bp, artificial sequence
  FLP recombinase recognition sequence
ttgaagttcctattccgagttcctattctctagaaagtataggaacttc

SEQ ID NO: 3 - DNA, 50 bp, artificial sequence
  FLP recombinase recognition sequence
ttgaagttcctattccgagttcctattcttcaaatagtataggaacttca

SEQ ID NO: 4 - DNA, 6055 bp, artificial sequence
  Vector pFRT-GIAMG
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcttgaagttcctattccgagttcctattctctagaaagtataggaacttcctcgagaccatgtaccgcttccttgtctgtgctctcgggcttctggggacagtcctcgctcagtcagtcgacagttatgtcggcagcgaaggccccatagcaaaggccggcgtccttgccaacattgggccgaacggctcaaaggcctctggtgcagccgccggcgtggtggtggctagccccagcaagtcggatcccgactattggtacacttggacgcgtgactcgtcactcgttttcaagtctctcattgatcagtacaccactggtatcgacagcacgagttcgttgaggtctctgatagacagtttcgttattgccgaggccaacattcagcaggtctctaatcccagcggcactcttactaccggcggcttgggagagccaaaattcaatgtcgatgaaactgcattcaccggtgcatggggtcgaccccagcgcgacggacctgcgctccgtgcgactgctttgatcacctacggtaactggctcttgtcaaacgggaacacgacctgggttaccagtacgctgtggccgatcatccagaacgatctcaactacgtcgttcagtactggaaccagaccaccttcgacctctgggaagaagtgaactcttcctcgttcttcaccactgcagtgcagcaccgtgccttgcgcgaaggcgcagcattcgctaccaagatcggtcagacctcctcggtcagcagctacacaacccaagcggcgaatctactttgctttttgcagtcttactggaaccccacttccggatatatcaccgctaacactggcggtggtcggtccggcaaggacgccaacaccctcttggcatccatccacacttacgaccccagcgcgggctgcgatgccacgaccttccagccctgctccgacaaagccctctcgaatctgaaggtttacgtcgactccttccgttctgtctactccatcaacagcggtattgcctctaacgccgctgtcgccactggtcgctacccggaagacagctaccagggcgggaacccatggtacctcactacgttcgccgtcgccgagcagctctatgacgccctcaatgtctgggctgctcagggctccctcaatgtcacctccatctccctccccttcttccagcagttctcctctagtgtcactgccggcacttacgcttcgagctccaccacttacacgactctgacctccgccattaagagcttcgcggatggattcgtcgctatcaacgcccagtacacgccgtccaacggtggcctcgctgagcagttcagcaggagcaacggcgctcccgtcagcgctgttgatttgacatggagctatgcatctgcattgaccgcgtttgaagcgaggaataatactcagttcgccggctggggcgcggtaggtttgactgtgccgacctcgtgctccagcaacagtggtggaggcggaggatcgactgtcgccgtgacgttcaacgtgaacgcccaaacggtttggggcgaaaacatctacatcactggctcggttgacgctctgagtaactggtctcccgacaacgccctcttgctctcgtctgccaactacccgacctggagcattaccgtgaatttacccgcgagcactgccattcagtataagtatatccgcaagaacaacggagctgtcacctgggaatccgatcccaacaacagcataactactccagccagcggctccgtgaccgagaatgacacttggcgttaattaattaataataaataacggattgtgtccgtaatcacacgtgcccgtggtgcgtacgataacgcatagtgtttttccctccacttaaatcgaagggttgtgtcttggatcgcgcgggtcaaatgtatatggttcatatacatccgcaggcacgtaataaagcgaggggttcgaatccccccgttacccccggtaggggcccagatccgggtgactgacacctggcggtagacaatcaatccatttcgctatagttaaaggatggggatgagggcaattggttatatgatcatgtatgtagtgggtgtgcataatagtagtgaaatggaagccaagtcatgtgattgtaatcgaccgacggaattgaggatatccggaaatacagacaccgtgaaagccatggtctttccttcgtgtagaagaccagacagacagtccctgatttacccttgcacaaagcactagaaaattagcattccatccttctctgcttgctctgctgatatcactgtcattcaatgcatagccatgagctcatcttagatccaagcacgtaattccatagccgaggtccacagtggagcagcaacattccccatcattgctttccccaggggcctcccaacgactaaatcaagagtatatctctaccgtccaatagatcgtcttcgcttcaaaatctttgacaattccaagagggtccccatccatcaaacccagttcaataatagccgagatgcatggtggagtcaattaggcagtattgctggaatgtcggggccagttggcccggtggtcattggccgcctgtgatgccatctgccactaaatccgatcattgatccaccgcccacgaggcgcgtctttgctttttgcgcggcgtccaggttcaactctctccactagtctagcggggttcaaatgcaaacaagtacaacacgcagcaaacgaagcagcccaccactgcgttgatgcccagtttgactgtccgaaatccaccggaaaggtggaaacatactatgtaacaatcagagggaagaaaaaatttttatcgacgaggcaggatagtgactgatggtggggtcatggtcgggtctccgagcgaaagagaaccaaggaaacaagatcaacgaggttggtgtacccaaaaggccgcagcaacaagagtcatcgcccaaaagtcaacagtctggaagagactccgccgtgcagattctgcgtcggtcccgcacatgcgtggtgggggcattacccctccatgtccaatgataagggcggcggtcgagggcttaagcccgcccactaattcgccttctcgcttgcccctccatataaggattccccctccttcccctcccacaacttttttccttctttctctcttcgtccgcatcagtacgtatatctttcccccatacctcctttcctactcttcttccattcattcaactcttctccttactgacatctgttttgctcagtacctctacgcgatcagccgtagtatctgagcaagcttctctacagaatctttctagtatcttacaaagaactacaaagttcgcaccattgaagttcctattccgagttcctattcttcaaatagtataggaacttcagcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 5 - DNA, 1731 bp, Gloeophyllum trabeum
atgtaccgcttccttgtctgtgctctcgggcttctggggacagtcctcgctcagtcagtcgacagttatgtcggcagcgaaggccccatagcaaaggccggcgtccttgccaacattgggccgaacggctcaaaggcctctggtgcagccgccggcgtggtggtggctagccccagcaagtcggatcccgactattggtacacttggacgcgtgactcgtcactcgttttcaagtctctcattgatcagtacaccactggtatcgacagcacgagttcgttgaggtctctgatagacagtttcgttattgccgaggccaacattcagcaggtctctaatcccagcggcactcttactaccggcggcttgggagagccaaaattcaatgtcgatgaaactgcattcaccggtgcatggggtcgaccccagcgcgacggacctgcgctccgtgcgactgctttgatcacctacggtaactggctcttgtcaaacgggaacacgacctgggttaccagtacgctgtggccgatcatccagaacgatctcaactacgtcgttcagtactggaaccagaccaccttcgacctctgggaagaagtgaactcttcctcgttcttcaccactgcagtgcagcaccgtgccttgcgcgaaggcgcagcattcgctaccaagatcggtcagacctcctcggtcagcagctacacaacccaagcggcgaatctactttgctttttgcagtcttactggaaccccacttccggatatatcaccgctaacactggcggtggtcggtccggcaaggacgccaacaccctcttggcatccatccacacttacgaccccagcgcgggctgcgatgccacgaccttccagccctgctccgacaaagccctctcgaatctgaaggtttacgtcgactccttccgttctgtctactccatcaacagcggtattgcctctaacgccgctgtcgccactggtcgctacccggaagacagctaccagggcgggaacccatggtacctcactacgttcgccgtcgccgagcagctctatgacgccctcaatgtctgggctgctcagggctccctcaatgtcacctccatctccctccccttcttccagcagttctcctctagtgtcactgccggcacttacgcttcgagctccaccacttacacgactctgacctccgccattaagagcttcgcggatggattcgtcgctatcaacgcccagtacacgccgtccaacggtggcctcgctgagcagttcagcaggagcaacggcgctcccgtcagcgctgttgatttgacatggagctatgcatctgcattgaccgcgtttgaagcgaggaataatactcagttcgccggctggggcgcggtaggtttgactgtgccgacctcgtgctccagcaacagtggtggaggcggaggatcgactgtcgccgtgacgttcaacgtgaacgcccaaacggtttggggcgaaaacatctacatcactggctcggttgacgctctgagtaactggtctcccgacaacgccctcttgctctcgtctgccaactacccgacctggagcattaccgtgaatttacccgcgagcactgccattcagtataagtatatccgcaagaacaacggagctgtcacctgggaatccgatcccaacaacagcataactactccagccagcggctccgtgaccgagaatgacacttggcgttaa

SEQ ID NO: 6 - protein, 576 aa, Gloeophyllum trabeum
MYRFLVCALGLLGTVLAQSVDSYVGSEGPIAKAGVLANIGPNGSKASGAAAGVVVASPSKSDPDYWYTWTRDSSLVFKSLIDQYTTGIDSTSSLRSLIDSFVIAEANIQQVSNPSGTLTTGGLGEPKFNVDETAFTGAWGRPQRDGPALRATALITYGNWLLSNGNTTWVTSTLWPIIQNDLNYVVQYWNQTTFDLWEEVNSSSFFTTAVQHRALREGAAFATKIGQTSSVSSYTTQAANLLCFLQSYWNPTSGYITANTGGGRSGKDANTLLASIHTYDPSAGCDATTFQPCSDKALSNLKVYVDSFRSVYSINSGIASNAAVATGRYPEDSYQGGNPWYLTTFAVAEQLYDALNVWAAQGSLNVTSISLPFFQQFSSSVTAGTYASSSTTYTTLTSAIKSFADGFVAINAQYTPSNGGLAEQFSRSNGAPVSAVDLTWSYASALTAFEARNNTQFAGWGAVGLTVPTSCSSNSGGGGGSTVAVTFNVNAQTVWGENIYITGSVDALSNWSPDNALLLSSANYPTWSITVNLPASTAIQYKYIRKNNGAVTWESDPNNSITTPASGSVTENDTWR

SEQ ID NO: 7 - DNA, 16642 bp, artificial sequence
  Plasmid p007
  misc_feature (11576)..(11576): n is a, c, g, or t
ttaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggagctccaccgcggcaacaggcagaatatcttccgaattcaatcgactgcgcgatgcaagttggctagcaacggcgtacaccttgggattatgcgctgctcaaccgatggtcagctatcaaacaaaatttgggaagatcgggctatactgacggtgacattatagtacggcaagctgagtgacatctacggtcgcaagccactgcttctttgggcatatgttttctttggcgtgggatgcattatcaggtagatactccctttttcttatacgctggtttgctggttcgtgctgacagctgtttccctagcggtattggtcgagacatggcgactgtcatattggggcgtgcaatcagcggaattgggggtgctggaacaatggcgatgggctctatcattatcacaggtaggctagcagcttatcaggttgaaagaactgtcactgaacataggcagatattgttcctcgtcgagatgttgcccattggcgggcgtacatcaatatcgcgatgactctgggtcgtagcgcaggaggcccaatcggcggatggctaaccgatacaatcggatggagatggtatgctttgcgcctttgtgaccgcttctctcactaaattgtggccaaggtcgtttattatccaaggccccttagccgctgtggcagctctgttggtgatatggaagctcaaactcgccaatccagtcactgagaagagcatccgccgtgtcgactttctcggaacattcctcctggccgtcggtattgttacaatcaccgttatcatggaccaagcagggcagtccttcgcatgggcatcattgtcaacagcaatccttgcaactctcagtctatcagcattcgtcgccttcgtccttgttgaactctacgtagcccctgaaccgattttcgaacttcgcatgttgcggaagccgaatgtgacgcccagttacctgatcggatcgctgcagatcaccgcccaagttggaatgatgttctccgtgccgttatattttcaggtgacatcgaaagcctctgccaccgtagctggagggcatctggttcctgcagtgatcggaaacacgcttggcggcttaatcgcgggagcctttatccgtcgcaccggccaattcaaggtcctcttgatccttgccggtctcgttgcgtccgtcgcctatctactcctcatccttcgctggaacggtcatactggattctgggagtccttgtacattattcccggtggtatgggtactggtttctgctctgcagctgcttttgtcagtatgacggcgtttttgatgccgcaggaagtggccatggcaacaggaggttacttcctattattcagcttcgccatgacggccggtgtcactgtcactaacagtctgctggggacggttttcaagcgccagatggaacagcacctgacgggtccaggagccaagaaggttggtatccccgcaccttttctgcgtcacttactaacgagtatatgaagatcatcgagcgcgcgctgtccgacaccagctatatcaacggtttgcagggtcatgtccgggatgtagtggtaaaaggatatgtgactggtctccgctacacttactgtaagtcgtttgaatcatgcatccaccgtccaccttattaacttggtgccagtattttccctcattctttcgctccttggatcggtcctcgcttggactgtacgaaaacaccaactatgaggaaccagcacggcagctgatagtatccgaaagctgcaaattgcttcatcgaggctggcattcgatagaagaaagaactatagacaactagtcttacaatatgacaattctctttgattaataaatgaaaataacacttgtgtcagcctaatagccgagtggcgggcatctctggcggcctcccgagcagcgtggatctggaagtgcgttgatcattattccccgaaaatgtagtacccagtaagtggtctagcggtggctatggtaggacatctatgcctaagctggagttctcattgaacgtgtaccggccgattgccctaaactctgattgagagccggaaacctcatctacctgatgctcaggggccatccaatagcttccgatagcattacagacagatggactcgtcttggcccacgggtctagaacagtcgccggaactgcctctatttgaaacggagctgaaccatgatacttaagcgtgccaagcggcgccgtttcccactggaacaaggagcaatagaattctgcagagattcttcattcaggctattcagcaattcggtttgtggagcggatcggggtccactgggtttagtctggggtttttctttgcccgcatgggctctagcacatgcacagcttgcagttgctgctacgctatctgggaaaacgaatggctattcaggagtttataaccaaaagagccggaaacaggctgattgccctctcacggggagacgttgtacttctgatccagaggctattaaccggacactacctataaaggaggtagcattcctttctgtccggctcccagattccaacaacccaactgacaggggatccaccatgccccagttcgatatcctctgcaagaccccccccaaggtcctcgtccgccagttcgtcgagcgcttcgagcgcccctccggcgagaagatcgccctctgcgccgccgagctcacctacctctgctggatgatcacccataacggcaccgccatcaagcgcgccaccttcatgtcctacaacaccatcatctccaactccctctccttcgatatcgtcaacaagtccctccagttcaagtacaagacccagaaggccaccatcctggaggcctccctcaagaagctcatccccgcctgggagttcaccatcatcccctactacggccagaagcatcagtccgatatcaccgatatcgtctcctccctccagctccagttcgagtcctccgaggaggccgataagggcaactcccattccaagaagatgctcaaggccctcctctccgagggcgagtccatctgggagatcaccgagaagatcctcaactccttcgagtacacctcccgcttcaccaagaccaagaccctctaccagttcctcttcctcgccaccttcatcaactgcggccgcttctccgatatcaagaacgtcgatcccaagtccttcaagctcgtccagaacaagtacctcggcgtcatcatccagtgcctcgtcaccgagaccaagacctccgtctcccgccatatctacttcttctccgcccgcggccgcatcgatcccctcgtctacctcgatgagttcctccgcaactccgagcccgtcctcaagcgcgtcaaccgcaccggcaactcctcctccaacaagcaggagtaccagctcctcaaggataacctcgtccgctcctacaacaaggccctcaagaagaacgccccctactccatcttcgccatcaagaacggccccaagtcccatatcggccgccatctcatgacctccttcctctccatgaagggcctcaccgagctcaccaacgtcgtcggcaactggtccgataagcgcgcctccgccgtcgcccgcaccacctacacccatcagatcaccgccatccccgatcattacttcgcactagtctcccgctactacgcctacgatcccatctccaaggagatgatcgccctcaaggatgagaccaaccccatcgaggagtggcagcatatcgagcagctcaagggctccgccgagggctccatccgctaccccgcctggaacggcatcatctcccaggaggtcctcgattacctctcctcctacatcaaccgccgcatctgagtcgagattatccaagggaatgacttaatgagtatgtaagacatgggtcataacggcgttcgaaacatatacagggttatgtttgggaatagcacacgaataataacgttaataggtaccaaagtccttgatacattagcacggtagaaaaagaataatacaacgagctgggaatattctttaatataaaactccaagaagagctggtgcggtggagcttgttttcgactctcagtaatatttcctcatatccaagcgcgctaggaggtggtcgaatacacatgtaggcgcttctctggatgcaaaagtcgtgccggacctgccgaaagactttgaagatgcgttcacgccatctaagttgcgtagataattcacaaaaagggatgtttgtttccggaatgtagcaaagagctgataggcaatagcctcactttcgtggcgcacgccgctcgttccatccatcctcgacaatggagcaaatgtcaaaatcgtaccgaaaatactttgctagcccgttaaattgccgtcgtcagccgttaaattaccgattaatcccgataaatttccgagatctccgttaaattgccgttcgcagccgttaaattaccggggacgaccgataaatttccgcgatgaattcatggtgttttgatcattttaaatttttatatggcgggtggtgggcaactcgcttgcgcgggcaactcgcttaccgattacgttagggctgatatttacgtaaaaatcgtcaagggatgcaagaccaaaccgttaaatttccggagtcaacagcatccaagcccaagtccttcacggagaaaccccagcgtccacatcacgagcgaaggaccacctctaggcatcggacgcaccatccaattagaagcagcaaagcgaaacagcccaagaaaaaggtcggcccgtcggccttttctgcaacgctgatcacgggcagcgatccaaccaacaccctccagagtgactaggggcggaaatttatcgggattaatttccactcaaccacaaatcacagtcgtccccggtaatttaacggctgcagacggcaatttaacggcttctgcgaatcgcttggattccccgcccctggccgtagagcttaaagtatgtcccttgtcgatgcgatgtatcacaacatataaatactggcaagggatgccatgcttggagcaacaatctcagaacaccaatatcaattcatctcgagtctgaactcagacaacacaaattcttcaaaattgaggatttagtcttgatcttgaagttcctattccgagttcctattctctagaaagtataggaacttcttaattaacctagccgttattactctaccgcaaggcaacaaccagctcacccctgaggcacgggtaccatgggttgagtggtatggggccatccagagtcacctgtggcagcatgagactgcactcgaagcagccatcaacccagccaatattctgggctttccatccttagatcacatttgagatataacccatttggtgagagacacttgtgccgttatacgtgtctagactggaaacgcaaccctgaagggattcttcctttgagagatggaagcgtgtcatatctcttcggttctacggcaggtttttttctgctctttcgtagcatggcatggtcacttcagcgcttatttacagttgctggtattgatttcttgtgcaaattgctatctgacacttattaggtcgagctattcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatggcctccgcgaccggctgcagaacagcgggcagttcggtttcaggcaggtcttgcaacgtgacaccctgtgcacggcgggagatgcaataggtcaggctctcgctgaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggcgacattgctgaggtgtaggatcgatccgtttaaactctgtgttagcttatagtcaggatgttggctcgacgagtgtaaactgggagttggcatgagggttatgtaggcttctttagccccgcatccccctcattctcctcattgatcccgggggagcggatggtgttgataagagactaattatagggtttagctggtgcctagctggtgattggctggcttcgccgaattttacgggccaagggaagctgcagaaccgcggcactggtaaacggtaattaagctatcagccccatgctaacgagtttaaattacgtgtattgctgataaacaccaacagagctttactgaaagatgggagtcacggtgtggcttccccactgcgattattgcacaagcagcgagggcgaacttgactgtcgtcgctgagcagcctgcagtcaaacatacatatatatcaaccgcgaagacgtctggccttgtagaacacgacgctccctagcaacacctgccgtgtcagcctctacggttgttacttgcattcaggatgctctccagcgggcgagctattcaaaatattcaaagcaggtatctcgtattgccaggattcagctgaagcaacaggtgccaaggaaatctgcgtcggttctcatctgggcttgctcggtcctggcgtagactagttgaagttcctattccgagttcctattcttcaaatagtataggaacttcatcagggagatgtaacaacgccaccttatgggactatcaagctgacgctggcttctgtgcagacaaactgcgcccacgagttcttccctgacgccgctctcgcgcaggcaagggaactcgatgaatactacgcaaagcacaagagacccgttggtccactccatggcctccccatctctctcaaagaccagcttcgagtcaaggtacaccgttgcccctaagtcgttagatgtccctttttgtcagctaacatatgccaccagggctacgaaacatcaatgggctacatctcatggctaaacaagtacgacgaaggggactcggttctgacaaccatgctccgcaaagccggtgccgtcttctacgtcaagacctctgtcccgcagaccctgatggtctgcgagacagtcaacaacatcatcgggcgcaccgtcaacccacgcaacaagaactggtcgtgcggcggcagttctggtggtgagggtgcgatcgttgggattcgtggtggcgtcatcggtgtaggaacggatatcggtggctcgattcgagtgccggccgcgttcaacttcctgtacggtctaaggccgagtcatgggcggctgccgtatgcaaagatggcgaacagcatggagggtcaggagacggtgcacagcgttgtcgggccgattacgcactctgttgagggtgagtccttcgcctcttccttcttttcctgctctataccaggcctccactgtcctcctttcttgctttttatactatatacgagaccggcagtcactgatgaagtatgttagacctccgcctcttcaccaaatccgtcctcggtcaggagccatggaaatacgactccaaggtcatccccatgccctggcgccagtccgagtcggacattattgcctccaagatcaagaacggcgggctcaatatcggctactacaacttcgacggcaatgtccttccacaccctcctatcctgcgcggcgtggaaaccaccgtcgccgcactcgccaaagccggtcacaccgtgaccccgtggacgccatacaagcacgatttcggccacgatctcatctcccatatctacgcggctgacggcagcgccgacgtaatgcgcgatatcagtgcatccggcgagccggcgattccaaatatcaaagacctactgaacccgaacatcaaagctgttaacatgaacgagctctgggacacgcatctccagaagtggaattaccagatggagtaccttgagaaatggcgggaggctgaagaaaaggccgggaaggaactggacgccatcatcgcgccgattacgcctaccgctgcggtacggcatgaccagttccggtactatgggtatgcctctgtgatcaacctgctggatttcacgagcgtggttgttccggttacctttgcggataagaacatcgataagaagaatgagagtttcaaggcggttagtgagcttgatgccctcgtgcaggaagagtatgatccggaggcgtaccatggggcaccggttgcagtgcaggttatcggacggagactcagtgaagagaggacgttggcgattgcagaggaagtggggaagttgctgggaaatgtggtgactccataggtaagctccgtggcgaaagcctgacgcaccggtagattcttggtgagcccgtatcatgacggcggcgggagctacatggccccgggtgatttattttttttgtatctacttctgacccttttcaaatatacggtcaactcatctttcactggagatgcggcctgcttggtattgcgatgttgtcagcttggcaaattgtggctttcgaaaacacaaaacgattccttagtagccatgcattttaagataacggaatagaagaaagaggaaattaaaaaaaaaaaaaaaacaaacatcccgttcataacccgtagaatcgccgctcttcgtgtatcccagtaccacggcaaaggtatttcatgatcgttcaatgttgatattgttcccgccagtatggctccacccccatctccgcgaatctcctcttctcgaacgcggtagtggcgcgccaattggtaatgacccatagggagacaaacagcataatagcaacagtggaaattagtggcgcaataattgagaacacagtgagaccatagctggcggcctggaaagcactgttggagaccaacttgtccgttgcgaggccaacttgcattgtctagagaatgcaatcataacagaaagtacagccagcgctgtgtcataaagaagtccagttgggaaacgaaagactagaatcaaactaaaagtaatccggccgatatggcttcacgtgcgaagtctcgccttgaggggacattgtccttgcaggtgattgaccattgcgttcatatggcgcgatgtttggtagtgtgggtgtagccggtgacctcacggaaggactaaaggccacatacccttctgagtgcctcttctcttcgtggtcggaactctcgaatgggtttttgacagttgcactcgtttggttgtggtcatttgaaggtctgcgttcggtcttctgttcgcgcaggcgagctgactgagggattgaaagctgcatagccatcgttggcatgcgttaattcgccaaagctcagcggcgaaacaggcctgacctctaatccatgcatctgctctgcactcgattgttcgtggtgtccttgcgaagaaagagaagccttggactcggatgactttctggacgaggtggtaggatcatcatgattgtaatgagactgtagcacatcatgcgaatcattcgacacacggtgtctgcccaagttgacgtcagcatcggtatgcatttcggtatggtcctcatggttctcagcatgtccctccagaggggactcatttccagcggaaggattataagcaacataattgtcatgtggctgcgacctttcgtgagactccgagtttgatctcactgtggactcatgggcgatatgcggctcatcatgatcttcgaatggagagaaatggttgaagtcggaggacacgggtgatttagcagcagggttgaatgcaacatagccagtctcgcgctcttcatgtgagctatatgagtcatgtggcctgtcatggtccagaggctccggatgctcatggctagattcatcgtgtgccgaaatcgcgtcactagcaaagggcgaggttgacacattggctgcaggactgaacgccacataaccactctcaggctcttcatgtgagttataggagctgtgcggcatatcatagtcctgaggttcacgatgctcatggctggattcatcgtgtgccgaaatagcgtgactagcaaaaggcgagggcgaagcattggttgccggactgaacgccacatagccgtccccattggctgaactgactggtgacaacgtcctacccatggcgtcggccgggccagcggcttggtgagagtgaagaccattagaagtagctggactgaacgatcgcagcgggtattcgttttcttgagctggataaggggctgcgccatgctggctgaaaggtgagaatgttcggggtgctgctctatcaccagggaaggcagacgctggagtcaaagaacgagtgttggatcaattgccggactgtatgaacggaaaggagtgctgattgtttaaatggcccgtagccttcgctaggacctcgtgattcggggaccgttggcccatacccaggagctggtgtaaaattggaacgcgacacgggtgtttggttgcgcagaatttgcggtgccggcgaggcgtgatcaatctggctgtaacctgggcctggggtgtagtttgagacaggtgtttgtgttcgtggcatttgtggcgctggcgacgctctgtcagtcggcccatatccaggcgccgaaggtgtgggcgtaaacccttgccgtgatttaattaagaattctcctgtaggcttgagagttcaaggaagaaacagtgcaattatctttgcgaacccaggngctggtgacggaattttcatagtcaagctatcagagtaaagaagaggagcatgtcaaagtacaattagagacaaatatatagtcgcgtggagccaagagcggattcctcagtctcgtaggtctcttgacgaccgttgatctgcttgatctcgtctcccgaaaatgaaaatagactctgctaagctattcttctgcttcgccggagcctgaagggcgtactagggttgcgaggtccaatgcattaatgcattgcagatgagctgtatctggaagaggtaaacccgaaacgcgttttattcttgttgacatggagctattaaatcactagaaggcactctttgctgcttggacaaatgaacgtatcttatcgagatcctgaacaccatttgtctcaactccggagctgacatcgacaccaacgatcttatatccagattcgtcaagctgtttgatgatttcagtaacgttaagtggatcgatccggatagcgcgggttccttccggtattgtctccttccgtgtttcagttagcctcccccatctcccgggcaaacgtgcgcgccaggtcgcatatcgtcggtatggagccgggggtggtgacgtgggtctggaccatcccggaggtaagttgcagcagggcgtcccggcagccggcgggcgattggtcgtaatccaggataaagacgtgcatggaacggaggcgtttggccaagacgtccaaggcccaggcaaacacgttatacaggtcgccgttgggggccagcaactcggggccccgaaacagggtaaataacgtgtccccgatatggggtcgtgggcccgcgttgctctggggctcggcaccctggggcggcacggccgtccccgaaagctgtccccagtcctcccgccacgacccgccgcactgcagataccgcaccgtattggcaagtagcccgtaaacgcggcgaatcgcagccagcatagccaggtccagccgctcgccggggcgctggcgtttggccaggcggtcgatgtgtctgtcctccggaagggccccaagcacgatgttggtgccgggcaaggtcggcgggatgagggccacgaacgccagcacggcctggggggtcatgctgcccataaggtaccgcgcggccgggtagcacaggagggcggcgatgggatggcggtcgaagatgagggtgagggccgggggcggggcatgtgagctcccagcctcccccccgatatgaggagccagaacggcgtcggtcacggcataaggcatgcccattgttatctgggcgcttgtcattaccaccgccgcgtccccggccgatatctcaccctggtcgaggcggtgttgtgtggtgtagatgttcgcgattgtctcggaagcccccagcacccgccagtaagtcatcggctcgggtacgtagacgatatcgtcgcgcgaacccagggccaccagcagttgcgtggtggtggttttccccatcccgtggggaccgtctatataaacccgcagtagcgtgggcattttctgctccgggcggacttccgtggcttcttgctgccggcgagggcgcaacgccgtacgtcggttgctatggccgcgagaacgcgcagcctggtcgaacgcagacgcgtgttgatggccggggtacgaagccatacgcgcttctacaaggcgctggccgaagaggtgcgggagtttcacgccaccaagatcgatctacagctggtggggagagcaggaaaatatggcaacaaatgttggactgacgcaacgaccttgtcaaccccgccgacacaccgggcggacagacggggcaaagctgcctaccagggactgagggacctcagcaggtcgagtgcagagcaccggatgggtcgactgccagcttgtgttcccggtctgcgccgctggccagctcctgagcggcctttccggtttcatacaccgggcaaagcaggagaggcacgatatttggacgccctacagatgccggatgggccaattagggagcttacgcgccgggtactcgctctacctacttcggagaaggtactatctcgtgaatcttttaccagatcggaagcaattggacttctgtacctaggttaatggcatgctatttcgccgacggctatacacccctggcttcacattctccttcgcttactgccggtgattcgatgaagctccatattctccgatgatgcaatagattcttggtcaacgaggggcacaccagcctttccacttcggggcggaggggcggccggtcccggattaataatcatccactgcacctcagagccgccagagctgtctggcgcagtggccgttattactcagcccttctctctgcgtccgtccgtctctccgcatgccagaaagagtcaccggtcactgtacagagctcaagcttcgattaactcgagggggggcccggtacccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattca

SEQ ID NO: 8 - DNA, 80 bp, artificial sequence
  Primer SpeI-FRTF3-amdS
tggcgtagactagttgaagttcctattccgagtcctattcttcaaatagtataggaacttcatcagggagatgtaacaac

SEQ ID NO: 9 - DNA, 22 bp, artificial sequence
  Primer amdS-F-probe
ctatggagtcaccacatttccc

SEQ ID NO: 10 - DNA, 6330 bp, artificial sequence
  Vector pFRT-BsAMG
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcttgaagttcctattccgagttcctattctctagaaagtataggaacttcctcgagaccatgtacttcgctcgccttctctccggactctctctcatcacctctgcggttctcgcccagaccgttgactcctatgtcagcactgagggccccatcgcgaaggcgggcctccttgcgaacattggtcccagtggttccaaatccaacggtgcccatgctggagttgtcgtcgctagccctagccaagtcaacccggactacatgtacacatgggtccgcgactcttccctcgtgttcaaggtcctcgtcgaccagctcgcttccggccaggacacctctgtccgtagcctgatcgatgcattcgtcgcagcggagtcggcgatgcagcacacccccaaccccagtggtgacatcaacactggtggtcttgctgagcccaagttcaacatcgacaccaccgccttcactggcgcatggggccgcccgcagcgagatggccctgctctccgttctaccaccgtcatcaactacgccaactacctccttgctaatggcaacaccactgcttgggtggtggccaacctctggcccatgatcaagctcgacttggactacatccagaacaactggaaccagtctaccttcgacctgtgggaggagctcaactcctcgtccttcttcaccaccgctgttcagcaccgtgctctccgcgagggtattactctcggcgcgaagctctcgaagactgccgataccgccaactatggcacccaggccggcaacatcctttgcttcctccagtcatactggaaccctaccgccaactacgtgacctccaacaccggtggtggacgctccggcaaggactccaactccgtccttacttccatccacactttcgaccccgatgccggatgtgatgctaccaccttccagccctgctccgacaaggccctgtccaacctcaaggtttacgtcgactcgttccgctccatctgggccatcaacgctggcgctgctgccaccgctcccgtcgctgtcggccgctaccccgaggacacttactacaacggtaacccctggtacctctctactttcgccgtcgccgagcagctctacgatgccatcatcgtctggaacaagcagggatccatccaggtcaccagcctttcccttcccttcttccagcagttcatctcgaccctcaagaccggcacgtacacccagagctccacccagttccagaccctcgtccctgccatcaaggcctgggctgacggcttcgtcgccatcaaccagaagtacactcctcctaacggtggtctcggtgaacagtacgacaaggtctctggcgaccctgtcagcgctgttgaccttacctggtcgtatgcttccgctctcaccgtcttcaacgctcgtgccggaaactactcccccggctggggtgccaagggcctgaccgtcccggcagtctgccagcccaacgctggccctcaagtccaggtgacgttcaaggttcaggccacgacggtctacggcgagaacatttacctcactggtgctaacccagctctgtcgaactggtcgcccgacaccgccctcgccatgtctgctgctaactaccctacctggtctgtcacagtcaccctcccggcgaacagcgtcatccagtacaagtacatcaggaagaacaacggcgccgtcacctgggagtctgaccccaacaaccaggtcaccatcccagcgagcggcacctacaccgtcaacgataactggcggtaattaattaataataaataacggattgtgtccgtaatcacacgtgcccgtggtgcgtacgataacgcatagtgtttttccctccacttaaatcgaagggttgtgtcttggatcgcgcgggtcaaatgtatatggttcatatacatccgcaggcacgtaataaagcgaggggttcgaatccccccgttacccccggtaggggcccagatccgggtgactgacacctggcggtagacaatcaatccatttcgctatagttaaaggatggggatgagggcaattggttatatgatcatgtatgtagtgggtgtgcataatagtagtgaaatggaagccaagtcatgtgattgtaatcgaccgacggaattgaggatatccggaaatacagacaccgtgaaagccatggtctttccttcgtgtagaagaccagacagacagtccctgatttacccttgcacaaagcactagaaaattagcattccatccttctctgcttgctctgctgatatcactgtcattcaatgcatagccatgagctcatcttagatccaagcacgtaattccatagccgaggtccacagtggagcagcaacattccccatcattgctttccccaggggcctcccaacgactaaatcaagagtatatctctaccgtccaatagatcgtcttcgcttcaaaatctttgacaattccaagagggtccccatccatcaaacccagttcaataatagccgagatgcatggtggagtcaattaggcagtattgctggaatgtcggggccagttggcccggtggtcattggccgcctgtgatgccatctgccactaaatccgatcattgatccaccgcccacgaggcgcgtctttgctttttgcgcggcgtccaggttcaactctctccactagtctagcggggttcaaatgcaaacaagtacaacacgcagcaaacgaagcagcccaccactgcgttgatgcccagtttgactgtccgaaatccaccggaaaggtggaaacatactatgtaacaatcagagggaagaaaaaatttttatcgacgaggcaggatagtgactgatggtggggtcatggtcgggtctccgagcgaaagagaaccaaggaaacaagatcaacgaggttggtgtacccaaaaggccgcagcaacaagagtcatcgcccaaaagtcaacagtctggaagagactccgccgtgcagattctgcgtcggtcccgcacatgcgtggtgggggcattacccctccatgtccaatgataagggcggcggtcgagggcttaagcccgcccactaattcgccttctcgcttgcccctccatataaggattccccctccttcccctcccacaacttttttccttctttctctcttcgtccgcatcagtacgtatatctttcccccatacctcctttcctactcttcttccattcattcaactcttctccttactgacatctgttttgctcagtacctctacgcgatcagccgtagtatctgagcaagcttctctacagaatctttctagtatcttacaaagaactacaaagttcgcaccaatgcctcaatcctgggaagaactggccgctgataagcgcgcccgcctcgcaaaaaccatccctgatgaatggaaagtccagacgctgcctgcggaagacagcgttattgatttcccaaagaaatcgggtatcctttcagaggccgaactgaagatcacagaggcctccgctgcagatcttgtgtccaagctggcggccggagagttgacctcggcggaagttacgctagcattctgtaaacgggcagtaatcgcccagcagttagtagggtcccctctacctcttgaagttcctattccgagttcctattcttcaaatagtataggaacttcacgcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 11 - DNA, 1722 bp, Byssocorticium
atgtacttcgctcgccttctctccggactctctctcatcacctctgcggttctcgcccagaccgttgactcctatgtcagcactgagggccccatcgcgaaggcgggcctccttgcgaacattggtcccagtggttccaaatccaacggtgcccatgctggagttgtcgtcgctagccctagccaagtcaacccggactacatgtacacatgggtccgcgactcttccctcgtgttcaaggtcctcgtcgaccagctcgcttccggccaggacacctctgtccgtagcctgatcgatgcattcgtcgcagcggagtcggcgatgcagcacacccccaaccccagtggtgacatcaacactggtggtcttgctgagcccaagttcaacatcgacaccaccgccttcactggcgcatggggccgcccgcagcgagatggccctgctctccgttctaccaccgtcatcaactacgccaactacctccttgctaatggcaacaccactgcttgggtggtggccaacctctggcccatgatcaagctcgacttggactacatccagaacaactggaaccagtctaccttcgacctgtgggaggagctcaactcctcgtccttcttcaccaccgctgttcagcaccgtgctctccgcgagggtattactctcggcgcgaagctctcgaagactgccgataccgccaactatggcacccaggccggcaacatcctttgcttcctccagtcatactggaaccctaccgccaactacgtgacctccaacaccggtggtggacgctccggcaaggactccaactccgtccttacttccatccacactttcgaccccgatgccggatgtgatgctaccaccttccagccctgctccgacaaggccctgtccaacctcaaggtttacgtcgactcgttccgctccatctgggccatcaacgctggcgctgctgccaccgctcccgtcgctgtcggccgctaccccgaggacacttactacaacggtaacccctggtacctctctactttcgccgtcgccgagcagctctacgatgccatcatcgtctggaacaagcagggatccatccaggtcaccagcctttcccttcccttcttccagcagttcatctcgaccctcaagaccggcacgtacacccagagctccacccagttccagaccctcgtccctgccatcaaggcctgggctgacggcttcgtcgccatcaaccagaagtacactcctcctaacggtggtctcggtgaacagtacgacaaggtctctggcgaccctgtcagcgctgttgaccttacctggtcgtatgcttccgctctcaccgtcttcaacgctcgtgccggaaactactcccccggctggggtgccaagggcctgaccgtcccggcagtctgccagcccaacgctggccctcaagtccaggtgacgttcaaggttcaggccacgacggtctacggcgagaacatttacctcactggtgctaacccagctctgtcgaactggtcgcccgacaccgccctcgccatgtctgctgctaactaccctacctggtctgtcacagtcaccctcccggcgaacagcgtcatccagtacaagtacatcaggaagaacaacggcgccgtcacctgggagtctgaccccaacaaccaggtcaccatcccagcgagcggcacctacaccgtcaacgataactggcggtaa

SEQ ID NO: 12 - protein, 573 aa, Byssocorticium
MYFARLLSGLSLITSAVLAQTVDSYVSTEGPIAKAGLLANIGPSGSKSNGAHAGVVVASPSQVNPDYMYTWVRDSSLVFKVLVDQLASGQDTSVRSLIDAFVAAESAMQHTPNPSGDINTGGLAEPKFNIDTTAFTGAWGRPQRDGPALRSTTVINYANYLLANGNTTAWVVANLWPMIKLDLDYIQNNWNQSTFDLWEELNSSSFFTTAVQHRALREGITLGAKLSKTADTANYGTQAGNILCFLQSYWNPTANYVTSNTGGGRSGKDSNSVLTSIHTFDPDAGCDATTFQPCSDKALSNLKVYVDSFRSIWAINAGAAATAPVAVGRYPEDTYYNGNPWYLSTFAVAEQLYDAIIVWNKQGSIQVTSLSLPFFQQFISTLKTGTYTQSSTQFQTLVPAIKAWADGFVAINQKYTPPNGGLGEQYDKVSGDPVSAVDLTWSYASALTVFNARAGNYSPGWGAKGLTVPAVCQPNAGPQVQVTFKVQATTVYGENIYLTGANPALSNWSPDTALAMSAANYPTWSVTVTLPANSVIQYKYIRKNNGAVTWESDPNNQVTIPASGTYTVNDNWR

SEQ ID NO: 13 - DNA, 283 bp, aspergillus nidulans
atgcctcaatcctgggaagaactggccgctgataagcgcgcccgcctcgcaaaaaccatccctgatgaatggaaagtccagacgctgcctgcggaagacagcgttattgatttcccaaagaaatcgggtatcctttcagaggccgaactgaagatcacagaggcctccgctgcagatcttgtgtccaagctggcggccggagagttgacctcggtggaagttacgctagcattctgtaaacgggcagcaatcgcccagcagttagtagggtcccctctacctc

SEQ ID NO: 14 - DNA, 17 bp, artificial sequence
  Primer M13 RV
caggaaacagctatgac

SEQ ID NO: 15 - DNA, 17 bp, artificial sequence
  Primer M13 M4
gttttcccagtcacgac

SEQ ID NO: 16 - DNA, 19 bp, artificial sequence
  Primer In fusion vector R
tatgcgttatcgtacgcac

SEQ ID NO: 17 - DNA, 17 bp, artificial sequence
  Primer M13 M4
gttttcccagtcacgac

SEQ ID NO: 18 - DNA, 27 bp, artificial sequence
  Primer M49X F
  misc_feature (16)..(17): n is a, c, g, or t
gtcaacccggactacnnktacacatgg

SEQ ID NO: 19 - DNA, 18 bp, artificial sequence
  Primer M49X R
gtagtccgggttgacttg

SEQ ID NO: 20 - DNA, 37 bp, artificial sequence
  Primer SOE-M4 F
gtactatctggcattggtacgttttcccagtcacgac

SEQ ID NO: 21 - DNA, 37 bp, artificial sequence
  Primer SOE-MR R
tggttatgatttcggcgatgcaggaaacagctatgac

SEQ ID NO: 22 - DNA, 20 bp, artificial sequence
  Primer SOE-F
gtactatctggcattggtac

SEQ ID NO: 23 - DNA, 20 bp, artificial sequence
  Primer SOE-R
tggttatgatttcggcgatg

SEQ ID NO: 24 - DNA, 20 bp, artificial sequence
  Primer insert rescue F
aatctcagaacaccaatatc

SEQ ID NO: 25 - DNA, 20 bp, artificial sequence
  Primer insert rescue R
aacactatgcgttatcgtac

SEQ ID NO: 26 - DNA, 11101 bp, artificial sequence
  Vector pDAu571
ttgaagttcctattccgagttcctattctctagaaagtataggaacttcagtacccgggtataagctagcttccgttaaattgccgtcgtcagccgttaaattaccgattaatcccgataaatttccgagatctccgttaaattgccgttcgcagccgttaaattaccggggacgaccgataaatttccgcgatgaattcatggtgttttgatcattttaaatttttatatggcgggtggtgggcaactcgcttgcgcgggcaactcgcttaccgattacgttagggctgatatttacgtaaaaatcgtcaagggatgcaagaccaaaccgttaaatttccggagtcaacagcatccaagcccaagtccttcacggagaaaccccagcgtccacatcacgagcgaaggaccacctctaggcatcggacgcaccatccaattagaagcagcaaagcgaaacagcccaagaaaaaggtcggcccgtcggccttttctgcaacgctgatcacgggcagcgatccaaccaacaccctccagagtgactaggggcggaaatttatcgggattaatttccactcaaccacaaatcacagtcgtccccggtaatttaacggctgcagacggcaatttaacggcttctgcgaatcgcttggattccccgcccctggccgtagagcttaaagtatgtcccttgtcgatgcgatgtatcacaacatataaatactggcaagggatgccatgcttggagtttccaactcaatttacctctatccacacttctcttccttcctcaatcctctatatacacaactggggatccaccatgttctcggcaggccacaagattaagggtacagtcgtcctcatgcctaaaaacgagttggaagtgaaccccgatggctccgcagtcgataacctcaacgcattcctcggacgttcggtgtcgctccagctcatctccgcgaccaaagccgacgcccacggtaagggaaaggtgggcaaggacacgttcttggaaggtatcaacacttcgctccctaccttgggagcaggagagtccgcattcaacattcacttcgagtgggacggttcgatgggcattcccggagcgttctatatcaagaactatatgcaggtggagttcttcttgaagtccttgaccttggaggcaatctcgaaccagggtaccatccgtttcgtgtgtaactcgtgggtctacaacaccaagctctacaaatccgtgcggatcttcttcgcgaaccacacttacgtcccttcggagacacctgcccctttggtgtcgtaccgcgaggaggaattgaagtccctccgtggtaacggtactggagaaaggaaggagtatgataggatctacgactacgacgtctataacgatttgggtaaccccgacaaatcggaaaagttggcacgtcctgtgttgggaggctcctccaccttcccctaccctcgacgcggccgcacgggacgcggtcccactgtcaccgatccgaacacagagaagcagggcgaagtcttctacgtgcccagggacgaaaacctcggccacttgaagtcgaaggatgcattggagattggaaccaagtccctctcccagatcgtccagcctgcattcgaatcggcgttcgatttgaaatcgacgcccatcgagttccactcgttccaggacgtccatgacttgtatgaaggtggtatcaaattgcctcgggacgtcatctccaccattatccccctccccgtgatcaaggaattgtaccgcaccgacggccagcatattctcaaattcccccagccgcacgtcgtccaggtctcgcagtccgcatggatgacagatgaggaattcgcgagggaaatgattgcaggtgtcaacccgtgtgtcatccgaggcttggaggagttccctcctaagtccaacctcgatcctgccatctatggagaccagtcctccaagattacagccgattccctcgatctcgacggttatactatggatgaagcactcggttccaggcgattgttcatgctcgattatcatgatatcttcatgccctatgtgcgccagatcaaccagttgaactcggcaaaaacatatgcaacgaggacgatcctcttcctccgagaagacggcacactcaagcctgtggcaatcgagctctcgctcccccattccgcaggcgatctctccgcagccgtgtcgcaggtggtgttgcctgcaaaagaaggagtggagtcgaccatctggctcttggccaaagcatatgtgattgtgaacgattcctgttatcaccagctcatgtcgcattggctcaacactcacgcggcaatggaacccttcgtgatcgccacgcaccggcacctctcggtgctccacccgatctacaagctcctcactccccactaccgtaacaacatgaacattaacgccttggcacggcagtcgttgatcaacgcgaacggcatcattgagacaacgttcctcccctccaagtactccgtcgaaatgtcgtccgcagtctacaaaaactgggtcttcaccgaccaggcgttgcctgccgacttgatcaaacgaggcgtcgcaatcaaagatccctccactcctcatggcgtccgcctcttgatcgaggactacccctacgcagcggacggattggaaatctgggcagccatcaagacctgggtgcaggaatacgtccctttgtactatgcgagggacgatgatgtcaaaaacgactcggaactccagcattggtggaaggaggcagtggaaaagggccatggagatctcaaggataaaccctggtggcctaagctccagaccttggaggacctcgtcgaagtgtgtttgatcattatctggatcgcatccgcgttgcatgcagccgtgaacttcggacagtatccctatggaggcctcatcatgaaccgtcccaccgcatccaggaggctcctccccgaaaaaggaacacccgaatacgaagaaatgatcaacaaccacgaaaaggcatacctccggaccatcacttccaaactcccgaccttgatctcgctctccgtgatcgagattttgtcgacacatgcgtcggacgaggtctatttgggtcagcgggataacccgcactggacatccgattccaaggccctccaggcgttccagaagttcggcaacaagctcaaggagatcgaggagaaactcgtgaggcggaacaacgacccttccctccagggaaaccggttgggacctgtccagctcccgtatacgttgctctacccctcctcggaagaaggcctcactttcaggggtatccccaactcgatttccatctgactcgagatctagagggtgactgacacctggcggtagacaatcaatccatttcgctatagttaaaggatggggatgagggcaattggttatatgatcatgtatgtagtgggtgtgcataatagtagtgaaatggaagccaagtcatgtgattgtaatcgaccgacggaattgaggatatccggaaatacagacaccgtgaaagccatggtctttccttcgtgtagaagaccagacagacagtccctgatttacccttgcacaaagcactagaaaattagcattccatccttctctgcttgctctgctgatatcactgtcattcaatgcatagccatgagctcatcttagatccaagcacgtaattccatagccgaggtccacagtggagcagcaacattccccatcattgctttccccaggggcctcccaacgactaaatcaagagtatatctctaccgtccaatagatcgtcttcgcttcaaaatctttgacaattccaagagggtccccatccatcaaacccagttcaataatagccgagatgcatggtggagtcaattaggcagtattgctggaatgtcggggccagttggccgggtggtcattggccgcctgtgatgccatctgccactaaatccgatcattgatccaccgcccacgaggcgcgtctttgctttttgcgcggcgtccaggttcaactctctcttaattaaatagcgacaagccgaacggcaccggcaggtacaatggttcgctgtacttgcttgcgcaagcgggtctttggggattgagcgcatttggtgttgcaaaggatttgatgtaaatgtagtcgacatcttagcacagaggggagagttgataaaatgtggtctgtttgaatgatagtcgggttcgtgacctatattcgtgatagtggagataggtctgcgcctatcttatcgggccggagcaaaaattccaccgcagcggggtgagttttcgttatacagccatcccacttccagcttcaaattgtcagtttaatccagcccaattcaatcattggagaaccggttttatgtcttcgaagtcccacctcccctacgcaattcgcgcaaccaaccatcccaaccctttaacatctaaactcttctccatcgccgaggagaagaaaaccaacgtcaccgtctccgcagacgttactacttccgccgagctcctcgatcttgctgaccgcctaggcccctatatcgcagttctgaaaacccacatcgacatcctcaccgatctcaccccgtcgaccctttcctcgctccaatccctcgcgacaaagcacaacttcctcatctttgaggaccgcaagttcatcgacatcggcaacaccgtgcaaaagcagtaccacggtggcgctctccgcatctccgaatgggcacacatcatcaactgcgccatcctgccgggcgaagggatcgtcgaggccctcgcacagacaaccaagtctcctgactttaaagacgcgaatcaacgaggtctcctgattcttgccgagatgacgagtaagggatctcttgcgacaggggagtacacggcacgctcggttgagtacgcgcggaagtataaggggtttgtgatgggattcgtgagtacaagggcgttgagtgaggtgctgcccgaacagaaagaggagagcgaggattttgtcgtctttacgactggggtgaatctgtcggataagggggataagctggggcagcagtatcagacacctgggtcggcggttgggcgaggtgcggactttatcattgcgggtaggggcatctataaggcggacgatccagtcgaggcggttcagaggtaccgggaggaaggctggaaagcttacgagaaaagagttggactttgagggtgactgacacctggcggtagacaatcaatccatttcgctatagttaaaggatggggatgagggcaattggttatatgatcatgtatgtagtgggtgtgcataatagtagtgaaatggaagccaagtcatgtgattgtaatcgaccgacggaattgaggatatccggaaatacagacaccgtgaaagccatggtctttccttcgtgtagaagaccagacagacagtccctgatttacccttgcacaaagcactagaaaattagcattccatccttctctgcttgctctgctgatatcactgtcattcaatgcatagccatgagctcatcttagatccaagcacgtaattccatagccgaggtccacagtggagcagcaacattccccatcattgctttccccaggggcctcccaacgactaaatcaagagtatatctctaccgtccaatagatcgtcttcgcttcaaaatctttgacaattccaagagggtccccatccatcaaacccagttcaataatagccgagatgcatggtggagtcaattaggcagtattgctggaatgtcggggccagttggccgggtggtcattggccgcctgtgatgccatctgccactaaatccgatcattgatccaccgcccacgaggcgcgtctttgctttttgcgcggcgtccaggttcaactctctcctctaggttgaagttcctattccgagttcctattcttcaaatagtataggaacttcaactagctagtgcatgcgtacgattttgacatttgctccattgtcgaggatggatggaacgagcggcgtgcgccacgaaagtgaggctattgcctatcagctctttgctacattccggaaacaaacatccctttttgtgaattatctacgcaacttagatggcgtgaacgcatcttcaaagtctttcggcaggtccggcacgacttttgcatccagagaagcgcctacatgtgtattcgaccacctcctagcgcgcttggatatgaggaaatattactgagagtcgaaaacaagctccaccgcaccagctcttcttggagttttatattaaagaatattcccagctcgttgtattattctttttctaccgtgctaatgtatcaaggactttggtacctattaacgttattattcgtgtgctattcccaaacataaccctgtatatgtttcgaacgccgttatgacccatgtcttacatactcattaagtcattcccttggataatctcgactcagatgcggcggttgatgtaggaggagaggtaatcgaggacctcctgggagatgatgccgttccaggcggggtagcggatggagccctcggcggagcccttgagctgctcgatatgctgccactcctcgatggggttggtctcatccttgagggcgatcatctccttggagatgggatcgtaggcgtagtagcgggagactagtgcgaagtaatgatcggggatggcggtgatctgatgggtgtaggtggtgcgggcgacggcggaggcgcgcttatcggaccagttgccgacgacgttggtgagctcggtgaggcccttcatggagaggaaggaggtcatgagatggcggccgatatgggacttggggccgttcttgatggcgaagatggagtagggggcgttcttcttgagggccttgttgtaggagcggacgaggttatccttgaggagctggtactcctgcttgttggaggaggagttgccggtgcggttgacgcgcttgaggacgggctcggagttgcggaggaactcatcgaggtagacgaggggatcgatgcggccgcgggcggagaagaagtagatatggcgggagacggaggtcttggtctcggtgacgaggcactggatgatgacgccgaggtacttgttctggacgagcttgaaggacttgggatcgacgttcttgatatcggagaagcggccgcagttgatgaaggtggcgaggaagaggaactggtagagggtcttggtcttggtgaagcgggaggtgtactcgaaggagttgaggatcttctcggtgatctcccagatggactcgccctcggagaggagggccttgagcatcttcttggaatgggagttgcccttatcggcctcctcggaggactcgaactggagctggagggaggagacgatatcggtgatatcggactgatgcttctggccgtagtaggggatgatggtgaactcccaggcggggatgagcttcttgagggaggcctccaggatggtggccttctgggtcttgtacttgaactggagggacttgttgacgatatcgaaggagagggagttggagatgatggtgttgtaggacatgaaggtggcgcgcttgatggcggtgccgttatgggtgatcatccagcagaggtaggtgagctcggcggcgcagagggcgatcttctcgccggaggggcgctcgaagcgctcgacgaactggcggacgaggaccttggggggggtcttgcagaggatatcgaactggggcatggtgctcagatactacggctgatcgcgtagaggtactgagcaaaacagatgtcagtaaggagaagagttgaatgaatggaagaagagtaggaaaggaggtatgggggaaagatatacgtactgatgcggacgaagagagaaagaaggaaaaaagttgtgggaggggaaggagggggaatccttatatggaggggcaagcgagaaggcgaattagtgggcgggcttaagccctcgaccgccgcccttatcattggacatggaggggtaatgcccccaccacgcatgtgcgggaccgacgcagaatctgcacggcggagtctcttccagactgttgacttttgggcgatgactcttgttgctgcggccttttgggtacaccaacctcgttgatcttgtttccttggttctctttcgctcggagacccgaccatgaccccaccatcagtcactatcctgcctcgtcgataaaaattttttcttccctctgattgttacatagtatgtttccacctttccggtggatttcggacagtcaaactgggcatcaacgcagtggtgggctgcttcgtttgctgcgtgttgtacttgtttgcatttgaaccccgcggtcgttcgagtccttaattggtccgctcccggtcaacacccaagcagctgtggcccggccgagtggcgcctgtctggtccacagtaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttttcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacc

SEQ ID NO: 27 - DNA, 49 bp, artificial sequence; identical to SEQ ID NO: 2
  FRT-F

SEQ ID NO: 28 - DNA, 50 bp, artificial sequence; identical to SEQ ID NO: 3
  FRT-F3

SEQ ID NO: 29 - DNA, 8299 bp, artificial sequence
  Plasmid pDAu703
taggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattggcctccatggccgcggccgcgctttgctaaaactttggttgatggaaggtatctggcgataaactccgacgacgtctagaagcaacaatcttatgcaaacgctcattggttcttttcgaccgcaacatccatcatgaaactggtattttgtctgtgtcagcagtctagaaccccttgccgggtattttagcatttcatttttctataaaaaggtaccagcatgtatggatcgtatcttccgtaccgtggttattaaatcccagcagaggccgataggcttaagaagtgaacatggcatggttaaggaagaagccattactgagtatatatggctagaataatcgctgggaaagatttatgcttccaagaggcgtaggacggtataccatacagtacggtatttatgaacaattcgataataccactccccaaagcgggagataggacacccgcctcaggcaccaaccaccccctttttcaactgtcagtggtgcacgtttccatcgagcataagcttggtaccctaaggataggccctaatcttatctacatgtgactgcatcgatgtgtttggtcaaaatgaggcatgtggctcaccccacaggcggagaaacgtgtggctagtgcatgacagtcccctccatagattcaatttaatttttcgcggcaattgtcgtgcagtttgtatctacatttcattccatatatcaagagttagtagttggacatcctgattattttgtctaattactgaaaactcgaagtactaacctactaataagccagtttcaaccactaagtgctcatttatacaatatttgcagaaccccgcgctacccctccatcgccaacatgtcttccaagtcgcaattgacctacagcgcacgcgctagcaagcaccccaatgcgctcgtaaagaagctcttcgaggttgccgaggccaagaaaaccaatgtcaccgtttccgccgacgtgacaaccaccaaagagctgctggatttggctgaccgtatgcgcaccggggttgaagttcctattccgagttcctattcttcaaatagtataggaacttcattaattaaaggagagagttgaacctggacgccgcgcaaaaagcaaagacgcgcctcgtgggcggtggatcaatgatcggatttagtggcagatggcatcacaggcggccaatgaccaccgggccaactggccccgacattccagcaatactgcctaattgactccaccatgcatctcggctattattgaactgggtttgatggatggggaccctcttggaattgtcaaagattttgaagcgaagacgatctattggacggtagagatatactcttgatttagtcgttgggaggcccctggggaaagcaatgatggggaatgttgctgctccactgtggacctcggctatggaattacgtgcttggatctaagatgagctcatggctatgcattgaatgacagtgatatcagcagagcaagcagagaaggatggaatgctaattttctagtgctttgtgcaagggtaaatcagggactgtctgtctggtcttctacacgaaggaaagaccatggctttcacggtgtctgtatttccggatatcctcaattccgtcggtcgattacaatcacatgacttggcttccatttcactactattatgcacacccactacatacatgatcatataaccaattgccctcatccccatcctttaactatagcgaaatggattgattgtctaccgccaggtgtcagtcaccctctagatctcgagctcgctagagtcgacctatggagtcaccacatttcccagcaacttccccacttcctctgcaatcgccaacgtcctctcttcactgagtctccgtccgataacctgcactgcaaccggtgccccatggtacgcctccggatcatactcttcctgcacgagggcatcaagctcactaaccgccttgaaactctcattcttcttatcgatgttcttatccgcaaaggtaaccggaacaaccacgctcgtgaaatccagcaggttgatcacagaggcatacccatagtaccggaactggtcatgccgtaccgcagcggtaggcgtaatcggcgcgatgatggcgtccagttccttcccggccttttcttcagcctcccgccatttctcaaggtactccatctggtaattccacttctggagatgcgtgtcccagagctcgttcatgttaacagctttgatgttcgggttcagtaggtctttgatatttggaatcgccggctcgccggatgcactgatatcgcgcattacgtcggcgctgccgtcagccgcgtagatatgggagatgagatcgtggccgaaatcgtgcttgtatggcgtccacggggtcacggtgtgaccggctttggcgagtgcggcgacggtggtttccacgccgcgcaggataggagggtgtggaaggacattgccgtcgaagttgtagtagccgatattgagcccgccgttcttgatcttggaggcaataatgtccgactcggactggcgccagggcatggggatgaccttggagtcgtatttccatggctcctgaccgaggacggatttggtgaagaggcggaggtctaacatacttcatcagtgactgccggtctcgtatatagtataaaaagcaagaaaggaggacagtggaggcctggtatagagcaggaaaagaaggaagaggcgaaggactcaccctcaacagagtgcgtaatcggcccgacaacgctgtgcaccgtctcctgaccctccatgctgttcgccatctttgcatacggcagccgcccatgactcggccttagaccgtacaggaagttgaacgcggccggcactcgaatcgagccaccgatatccgttcctacaccgatgacgccaccacgaatcccaacgatcgcaccctcaccaccagaactgccgccgcacgaccagttcttgttgcgtgggttgacggtgcgcccgatgatgttgttgactgtctcgcagaccatcagggtctgcgggacagaggtcttgacgtagaagacggcaccggctttgcggagcatggttgtcagaaccgagtccccttcgtcgtacttgtttagccatgagatgtagcccattgatgtttcgtagccctggtggcatatgttagctgacaaaaagggacatctaacgacttaggggcaacggtgtaccttgactcgaagctggtctttgagagagatggggaggccatggagtggaccaacgggtctcttgtgctttgcgtagtattcatcgagttcccttgcctgcgcgagagcggcgtcagggaagaactcgtgggcgcagtttgtctgcacagaagccagcgtcagcttgatagtcccataaggtggcgttgttacatctccctgagaggtagaggggaccctactaactgctgggcgattgctgcccgtttacagaatgctagcgtaacttccaccgaggtcaactctccggccgccagcttggacacaagatctgcagcggaggcctctgtgatcttcagttcggcctctgaaaggatcaccgatttctttgggaaatcaataacgctgtcttccgcaggcagcgtctggactttccattcatcagggatggtttttgcgaggcgggcgcgcttatcagcggccagttcttcccaggattgaggcatgtgcatgcaatgtgtgtttatgtggaagtaagatacgacgagtttgattgagaaaagacagggtgattgtcaagttcagtatggaagaaagagtagaagaagatcagacgacagggaagagcgatgacataaaaggtggaagacggaagaaaaacgaaccaaatcaatcccactctatggcgggggttggactgcctgaggccggcactggtggggcttatcgataagttctcgtcaccggatgcaatgcgctgtcaactgctgacttggccctgaacatcctgtcctctacagatccatactatacaatgatcccagttatagtgcggtaaggtgcatatcatatctcattctcatgactcattcgacttttttttagagaaagtacatacgtggaacatacactaaacgcaacaggtcgcgacaacactggtatacaaaacggtccccggtgaatgacgttattagtgtctatcccccactcacacccgaaaagaataatagaaactaacagaaaaagcggcccgaggataagaggaacattcaaacagaaggggaatcataaaaaccgaaaaatgcaaggaaaagagaactcaaatcaataattttcataatactgtcgagagtaatacggaccagcgtctctcagggacatgcgtcggcgcaaggcatcatccaatctctcatctaacacatccagcattcgtgttcgatagtctaactgcttctctcggcgctcaagtcttgcttcccgatcatcgagttaattaagaagttcctatactttctagagaataggaactcggaataggaacttcaaggtaccgagctctatcctcaataccctattttccacgattccattgtcatatccaattccgttttcttttcttgttttcccctcatccaatcccgtccatcatttactcctttttcttgtgaatgcaagtggcactaagaaatccaacccccagacaaattttcctactcaggaacacaaaaacctcgtttctgctcccttctcgtacttcattcctatcgtctcggaatttcctcaacaaccctttccgactttgcgacagcgtcgcgattccagacttatgtgttctcgttcctactgtcgttaccagtctatttattccgaaacctctgatcgctgaatttcacacacaacacccccccgttgatgctggtggagaatccgtagcgtcaagagttgaattcactccatgttgtaacgaagtccacgaattgagacgattgatgattacaaccccgcgatcgcctatcgacgattcgacgagatgccattctcatcctcctcatcctcctccacccccgaggtgtctaccaccccgctcgcagattacttctggatcgcaggtgtcgatggcgcggaaatcttagagactttccaaagactcggcgacgaatacagggcaaacagtgccaccgctcctggccccgctcttgcggacacgatcgaggaagatgcggacgcggaggaggcacacgacccccgtctggactccctctctcgacccaattccatggctgggggccgcaattccttccagcggttctcaatgcgctcaggagactccagtgagtccagtgggaatggtaccagcagcaaccggagcagtctgaccatcaagggtaatcagtcgcccagagggtcgtcgtttctagaagatttcgactttgacaaggccctgttcaagtttgcaaacgagcgggagtcgttcctgtcggatctgagtctcagtgccggagcaatcactcccacctcccgtcctaggtccaggttacgtacacagaagattgtctccgaggaaagtccctcccagccatccagcttgcttcgatcaggcattggtagtgtgcggcgtcatatggcattcagagacatgaatagtatgaaacggcagccgtcagttgctcgtcgcggccgcagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttttcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaa

SEQ ID NO: 30 - DNA, 8614 bp, artificial sequence
  Plasmid pDAU724
gaattcgagctcggtaccttgaagttcctattccgagttcctattctctagaaagtataggaacttcagtacccgggtataagctagcttccgttaaattgccgtcgtcagccgttaaattaccgattaatcccgataaatttccgagatctccgttaaattgccgttcgcagccgttaaattaccggggacgaccgataaatttccgcgatgaattcatggtgttttgatcattttaaatttttatatggcgggtggtgggcaactcgcttgcgcgggcaactcgcttaccgattacgttagggctgatatttacgtaaaaatcgtcaagggatgcaagaccaaaccgttaaatttccggagtcaacagcatccaagcccaagtccttcacggagaaaccccagcgtccacatcacgagcgaaggaccacctctaggcatcggacgcaccatccaattagaagcagcaaagcgaaacagcccaagaaaaaggtcggcccgtcggccttttctgcaacgctgatcacgggcagcgatccaaccaacaccctccagagtgactaggggcggaaatttatcgggattaatttccactcaaccacaaatcacagtcgtccccggtaatttaacggctgcagacggcaatttaacggcttctgcgaatcgcttggattccccgcccctggccgtagagcttaaagtatgtcccttgtcgatgcgatgtatcacaacatataaatactggcaagggatgccatgcttggagtttccaactcaatttacctctatccacacttctcttccttcctcaatcctctatatacacaactggggatccaccatgaggagctcccttgtgctgttctttgtctctgcgtggacggccttggccagtcctattcgtcgagaggtctcgcaggatctgtttaaccagttcaatctctttgcacagtattctgcagccgcatactgcggaaaaaacaatgatgccccagctggtacaaacattacgtgcacgggaaatgcctgccccgaggtagagaaggcggatgcaacgtttctctactcgtttgaagactctggagtgggcgatgtcaccggcttccttgctctcgacaacacgaacaaattgatcgtcctctctttccgtggctctcgttccatagagaactggatcgggaatcttaacttcgacttgaaagaaataaatgacatttgctccggctgcaggggacatgacggcttcacttcgtcctggaggtctgtagccgatacgttaaggcagaaggtggaggatgctgtgagggagcatcccgactatcgcgtggtgtttaccggacatagcttgggtggtgcattggcaactgttgccggagcagacctgcgtggaaatgggtatgatatcgacgtgttttcatatggcgccccccgagtcggaaacagggcttttgcagaattcctgaccgtacagaccggcggaacactctaccgcattacccacaccaatgatattgtccctagactcccgccgcgcgaattcggttacagccattctagcccagagtactggatcaaatctggaacccttgtccccgtcacccgaaacgatatcgtgaagatagaaggcatcgatgccaccggcggcaataaccagcctaacattccggatatccctgcgcacctatggtacttcgggttaattgggacatgtctttagtggccggcgcggctgggtcgactctagcgagctcgagatctagagggtgactgacacctggcggtagacaatcaatccatttcgctatagttaaaggatggggatgagggcaattggttatatgatcatgtatgtagtgggtgtgcataatagtagtgaaatggaagccaagtcatgtgattgtaatcgaccgacggaattgaggatatccggaaatacagacaccgtgaaagccatggtctttccttcgtgtagaagaccagacagacagtccctgatttacccttgcacaaagcactagaaaattagcattccatccttctctgcttgctctgctgatatcactgtcattcaatgcatagccatgagctcatcttagatccaagcacgtaattccatagccgaggtccacagtggagcagcaacattccccatcattgctttccccaggggcctcccaacgactaaatcaagagtatatctctaccgtccaatagatcgtcttcgcttcaaaatctttgacaattccaagagggtccccatccatcaaacccagttcaataatagccgagatgcatggtggagtcaattaggcagtattgctggaatgtcggggccagttggccgggtggtcattggccgcctgtgatgccatctgccactaaatccgatcattgatccaccgcccacgaggcgcgtctttgctttttgcgcggcgtccaggttcaactctctcttaattaatgtacattagtgataccccactctaagaaaatagaccaatctccagctgcaccttcagacactccggtacaaattctcgtctatgttggagattgttgtgactttgaaacatgacccttgaccctgattttgaatttgtccatatatcgaggcaggtgtcttattcgtacggagagggtatctgtcgtagacacatagtagtagtcatttcgagtgctgaatttataaatcgcatcatacttgcgacatactgccataaaaggagtacgtatccaccactacttattgcgcaccaacacgcttcaggtatgcatcccatccctccttctggtactgcttcgccgcctccacgggatcaggagcagcataaattccacggccagcaataataaagtcggcaccgcgtccaacagccgactcaggagtttggtactgctgtcccagcttgtcacccttcgaggagaggttgacacctgtcgtgaagacgacaaaatcttcctcctccgaaggcgagctaacttcagactgaacctcgccaaggtgacgtgtcgagacgaatcccatcacaaacttcttatacttccgagcatagtcaacagaagaagtagtatattgaccggtagccaaagatcccttggaggtcatctccgcaaggatcaaaaggcccctctcggagccgtaggggaagtcctcggccgaagcagtctgggccagagcctcgacgataccctcaccgggcagaatactgcagttgatgatgtgggcccactcagagatacgcagagtgccgccatggtactgcttttggactgtgtttccgatatcgatgaacttgcgatcttcgaagatgaggaaattgtgcttctctgcaagggccttcagaccggtgatggtttcttcgctgaaatcggagaggatatcgatgtgagttttgatcacggcaatgtacggaccgagtcctgttatataatccaccattaaccattactagatcacatgtaagtggcattgaagttcctatactatttgaagaataggaactcggaataggaacttcaacgtacgattttgacatttgctccattgtcgaggatggatggaacgagcggcgtgcgccacgaaagtgaggctattgcctatcagctctttgctacattccggaaacaaacatccctttttgtgaattatctacgcaacttagatggcgtgaacgcatcttcaaagtctttcggcaggtccggcacgacttttgcatccagagaagcgcctacatgtgtattcgaccacctcctagcgcgcttggatatgaggaaatattactgagagtcgaaaacaagctccaccgcaccagctcttcttggagttttatattaaagaatattcccagctcgttgtattattctttttctaccgtgctaatgtatcaaggactttggtacctattaacgttattattcgtgtgctattcccaaacataaccctgtatatgtttcgaacgccgttatgacccatgtcttacatactcattaagtcattcccttggataatctcgactcagatgcggcggttgatgtaggaggagaggtaatcgaggacctcctgggagatgatgccgttccaggcggggtagcggatggagccctcggcggagcccttgagctgctcgatatgctgccactcctcgatggggttggtctcatccttgagggcgatcatctccttggagatgggatcgtaggcgtagtagcgggagactagtgcgaagtaatgatcggggatggcggtgatctgatgggtgtaggtggtgcgggcgacggcggaggcgcgcttatcggaccagttgccgacgacgttggtgagctcggtgaggcccttcatggagaggaaggaggtcatgagatggcggccgatatgggacttggggccgttcttgatggcgaagatggagtagggggcgttcttcttgagggccttgttgtaggagcggacgaggttatccttgaggagctggtactcctgcttgttggaggaggagttgccggtgcggttgacgcgcttgaggacgggctcggagttgcggaggaactcatcgaggtagacgaggggatcgatgcggccgcgggcggagaagaagtagatatggcgggagacggaggtcttggtctcggtgacgaggcactggatgatgacgccgaggtacttgttctggacgagcttgaaggacttgggatcgacgttcttgatatcggagaagcggccgcagttgatgaaggtggcgaggaagaggaactggtagagggtcttggtcttggtgaagcgggaggtgtactcgaaggagttgaggatcttctcggtgatctcccagatggactcgccctcggagaggagggccttgagcatcttcttggaatgggagttgcccttatcggcctcctcggaggactcgaactggagctggagggaggagacgatatcggtgatatcggactgatgcttctggccgtagtaggggatgatggtgaactcccaggcggggatgagcttcttgagggaggcctccaggatggtggccttctgggtcttgtacttgaactggagggacttgttgacgatatcgaaggagagggagttggagatgatggtgttgtaggacatgaaggtggcgcgcttgatggcggtgccgttatgggtgatcatccagcagaggtaggtgagctcggcggcgcagagggcgatcttctcgccggaggggcgctcgaagcgctcgacgaactggcggacgaggaccttggggggggtcttgcagaggatatcgaactggggcatggtgctcagatactacggctgatcgcgtagaggtactgagcaaaacagatgtcagtaaggagaagagttgaatgaatggaagaagagtaggaaaggaggtatgggggaaagatatacgtactgatgcggacgaagagagaaagaaggaaaaaagttgtgggaggggaaggagggggaatccttatatggaggggcaagcgagaaggcgaattagtgggcgggcttaagccctcgaccgccgcccttatcattggacatggaggggtaatgcccccaccacgcatgtgcgggaccgacgcagaatctgcacggcggagtctcttccagactgttgacttttgggcgatgactcttgttgctgcggccttttgggtacaccaacctcgttgatcttgtttccttggttctctttcgctcggagacccgaccatgaccccaccatcagtcactatcctgcctcgtcgataaaaattttttcttccctctgattgttacatagtatgtttccacctttccggtggatttcggacagtcaaactgggcatcaacgcagtggtgggctgcttcgtttgctgcgtgttgtacttgtttgcatttgaaccccgcggtcgttcgagtccttaattggtccgctcccggtcaacacccaagcagctgtggcccggccgagtggcgcctgtctggtccacagtaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttttcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagt

SEQ ID NO: 31 - DNA, 876 bp, artificial sequence
  Codon-optimized VaXET16 open reading frame
atgggctcgtccctctggacttgtttgatcctcctctccttggcatcggcatccttcgcagcgaaccctcgaactccgatcgatgtgcctttcggacggaactacgtgccgacatgggcattcgaccacattaagtatttgaacggaggctcggagatccagttgcatctcgacaagtacaccggcactggtttccagtcgaagggctcctacttgttcggacatttctccatgtacatcaaattggtgcctggtgactcggcaggaactgtcaccgcattctacctctcgtcgacaaacgcagagcatgacgaaatcgacttcgagttcctcggcaacaggacaggacagccgtacatcctccagaccaacgtcttcacaggaggcaaaggtgatcgggaacagcggatctacttgtggttcgatcccacaacccagtaccataggtactcggtgctctggaacatgtatcagatcgtcttctacgtcgacgattatccgatccgagtgttcaagaactccaacgacttgggcgtcaaattccccttcaaccagcccatgaagatttacaactcgttgtggaacgccgacgattgggcaaccaggggtggtctcgagaagacagattggtcgaaagcacctttcatcgcgtcgtacaagggtttccacatcgacggatgtgaagcctccgtgaacgccaagttctgtgacacccagggcaaacgatggtgggatcagccggaattccgggatttggatgcagcccagtggcagaagctcgcgtgggtcaggaacaagtacaccatctataactactgtaccgatcggaaacgatattcgcaggtgcctcccgagtgtacacgcgatagggacatc

SEQ ID NO: 32 - protein, 292 aa, vigna angularis
MGSSLWTCLILLSLASASFAANPRTPIDVPFGRNYVPTWAFDHIKYLNGGSEIQLHLDKYTGTGFQSKGSYLFGHFSMYIKLVPGDSAGTVTAFYLSSTNAEHDEIDFEFLGNRTGQPYILQTNVFTGGKGDREQRIYLWFDPTTQYHRYSVLWNMYQIVFYVDDYPIRVFKNSNDLGVKFPFNQPMKIYNSLWNADDWATRGGLEKTDWSKAPFIASYKGFHIDGCEASVNAKFCDTQGKRWWDQPEFRDLDAAQWQKLAWVRNKYTIYNYCTDRKRYSQVPPECTRDRDI

SEQ ID NO: 33 - DNA, 50 bp, artificial sequence
  Primer 615726 (sense)
accgggaggaaggctggaaagcttacgagaaaagagttggactttgaggg

SEQ ID NO: 34 - DNA, 40 bp, artificial sequence
  Primer 615728 (antisense)
tgagcgaggaagcggaagagcgcccaatacgcaaaccgcc

SEQ ID NO: 35 - DNA, 40 bp, artificial sequence
  Primer 615729 (sense)
tgcgtattgggcgctcttccgcttcctcgctcactgactc

SEQ ID NO: 36 - DNA, 68 bp, artificial sequence
  Primer 615731 (antisense)
tatactttctagagaataggaactcggaataggaacttcaaggaacaacactcaaccctatctcggtc

SEQ ID NO: 37 - DNA, 67 bp, artificial sequence
  Primer 615730 (sense)
tccgagttcctattctctagaaagtataggaacttcgcatttatcagggttattgtctcatgagcgg

SEQ ID NO: 38 - DNA, 41 bp, artificial sequence
  Primer 615611 (antisense)
tctagatctcgagtcagatgtccctatcgcgtgtacactcg

SEQ ID NO: 39 - DNA, 45 bp, artificial sequence
  Primer 615610 (sense)
acacgcgatagggacatctgactcgagatctagagggtgactgac

SEQ ID NO: 40 - DNA, 40 bp, artificial sequence
  Primer 615727 (antisense)
aactcttttctcgtaagctttccagccttcctcccggtac

SEQ ID NO: 41 - DNA, 80 bp, Artificial sequence
  Forward primer
cgaattctgcattgaagttcctattccgagttcctattcttcaaatagtataggaacttcagatatccatcacactggcg

SEQ ID NO: 42 - DNA, 80 bp, Artificial sequence
  Reverse primer
gccagtgtgatggatatctgaagttcctatactatttgaagaataggaactcggaataggaacttcaatgcagaattcgc

SEQ ID NO: 43 - DNA, 63 bp, Artificial sequence
  Forward primer
atatccatcacactggcggccgctcaactctctcctctaggttgaagttcctattccgagttc

SEQ ID NO: 44 - DNA, 45 bp, Artificial sequence
  Reverse primer
aggatgcatgctcgagcatgcactagctagttgaagttcctatac

SEQ ID NO: 45 - DNA, 45 bp, Artificial sequence
  Forward primer
caccctctgtgtattgcaccatgccccagttcgatatcctctgca

SEQ ID NO: 46 - DNA, 43 bp, Artificial sequence
  Reverse primer
aaactctaggatgcatgcaagtgaggctattgcctatcagctc

SEQ ID NO: 47 - DNA, 38 bp, Artificial sequence
  Forward primer
catcacactggcggccgcgaattctaggctaggtatgc

SEQ ID NO: 48 - DNA, 20 bp, Artificial sequence
  Reverse primer
ggtgcaatacacagagggtg

SEQ ID NO: 49 - DNA, 39 bp, Artificial sequence
  Forward primer
accgcggactgcgcaccatgagattcggttggctcgagg

SEQ ID NO: 50 - DNA, 38 bp, Artificial sequence
  Reverse primer
ttcgccacggagcttactagtagacacggggcagaggc

SEQ ID NO: 51 - DNA, 41 bp, Artificial sequence
  Forward primer
cgtgtttcttcccattcgcatgcgacctcgtggtcattgac

SEQ ID NO: 52 - DNA, 49 bp, Artificial sequence
  Reverse primer
gctttgacgttacattgacgtacttataagcggccgccagtgtgatgga

SEQ ID NO: 53 - DNA, 49 bp, Artificial sequence
  Forward primer
tccatcacactggcggccgcttataagtacgtcaatgtaacgtcaaagc

SEQ ID NO: 54 - DNA, 48 bp, Artificial sequence
  Reverse primer
tgcagaggatatcgaactggggcattttgtatctgcgaattgagcttg

SEQ ID NO: 55 - DNA, 48 bp, Artificial sequence
  Forward primer
caagctcaattcgcagatacaaaatgccccagttcgatatcctctgca

SEQ ID NO: 56 - DNA, 41 bp, Artificial sequence
  Reverse primer
gctgtttaaactctaggatgcatgcaagtgaggctattgcc

SEQ ID NO: 57 - DNA, 41 bp, Artificial sequence; identical to SEQ ID NO: 51
  Forward primer

SEQ ID NO: 58 - DNA, 48 bp, Artificial sequence; identical to SEQ ID NO: 54
  Reverse primer

SEQ ID NO: 59 - DNA, 28 bp, Artificial sequence
  Primer 1208187
gattgagttgaaactgcctaagatctcg

SEQ ID NO: 60 - DNA, 63 bp, Artificial sequence
  Primer 1208194
ctatactttctagagaataggaactcggaataggaacttcaaggtgcgcagtccgcggttgac

SEQ ID NO: 61 - DNA, 62 bp, Artificial sequence
  Primer 1208195
ctattccgagttcctattctctagaaagtataggaacttcggcgttgttacatctccctgag

SEQ ID NO: 62 - DNA, 40 bp, Artificial sequence
  Primer 1208196
gcgtcaggctttcgccacgtctacgccaggaccgagcaag

SEQ ID NO: 63 - DNA, 20 bp, Artificial sequence
  Primer 069134
cgcaatctatcgaatagcag

SEQ ID NO: 64 - DNA, 25 bp, Artificial sequence
  Primer 067947
ctacatcgaagctgaaagcacgaga

SEQ ID NO: 65 - DNA, 31 bp, Artificial sequence
  Primer 069860
cttctatcttgggatgcttcacgatacgtga

SEQ ID NO: 66 - DNA, 25 bp, Artificial sequence
  Primer 069861
cgcgcccttgaatatcggagaaggt

SEQ ID NO: 67 - DNA, 22 bp, Artificial sequence
  Primer 0610249
gagaacacagtgagaccatagc

SEQ ID NO: 68 - DNA, 22 bp, Artificial sequence
  Primer 0610250
tctcaacccaatcagcaacatg

SEQ ID NO: 69 - DNA, 3021 bp, Aspergillus niger
atgcgccacagcatcggattggcagcggccctactggcaccaaccctacctgtagcgttgggtcaatatattcgagacttaagcaccgagaaatggactctcagtagtcgagccttgaatcggacagtacctgctcaatttccatcgcaggttcacttagatctactaagggccggagtgattggtgagtactatttggaagtcaagtcctgtgagtatacaaccgctaacagcctcaatagatgatccgtaagtgacttcatctgccatggatgagaattgaatcgcactaaatattgctggagataccatggtttgaacgatttcaatcttcgctggatcgctgctgccaactggacttataccagtcaacccatcaaaggcctgtgagtcgctgtgaagtttgtgcaatgtcgttcgacaatactaagaaccaatagcctggacaattacgactcaacttggctcgtgtttgacggactggacactttcgcaacaatctcattctgtgggcagcaaatcgcatccacggacaatcagtttcgccagtatgcgttcgatgtatccaccgcactagggtcctgcaaaggagatcctgttctgagcatcaactttggaagcgcaccgaatattgttgatgctatcgcacaggactctaattcgcaaagtaagtttcagaggtgggggactgccgaagttgttacatgctaattgtatatagaatggcccgatgacgtccaactcacctacgagtacccaaatcggtggtttatgcgcaaagaacaatcggacttcggatgggattggggtccagcatttgcccctgcaggtccatggaagcctgcatatattgttcagctagacaagaaagaaagtgtctatgtcctgaacacggatttggatatataccgaaagggccaaattaactaccttccgccagaccagagccaaccttgggtcgtcaacgctagcattgacattttgggtccactacctaccaaaccaaccatgtcgattgaagtgcgcgatactcattctggcacgattcttacttcgcggactctgaacaatgtcagtgtggctggtaatgccataactggtgtcaccgttctcgacgggctgaccccgaaactgtggtggccgcaaggcctcggtgatcagaacctctacaatgtttctatcactgtccaaagtagaggaaaccagaccgtggccagtgtgaacaaacggacgggcttccgcaccatttttctcaaccagcgcaacattactgaagcacagcgtgcgcaaggaatcgcccctggagcaaactggcactttgaagtcaacggtcatgagttctacgcaaaaggatcgaaccttatcccaccagacagtttctggacccgtgttacagaagagaagatgtcacggctattcgatgcagtggtcgttggaaaccagaatatgctccgtgtctggtcctccggcgcgtacctgcatgactacatctatgatctggccgatgaaaagggcattctcttatggagcgagttcgagttcagtgacgctttatatccctccgacgacgctttcctcgagaacgttgctgctgagatagtatacaatgttcgacgagtgaaccaccatccctccttggctctatgggctggcggaaatgaaatcgaatccttgatgctcccacgtgtcaaagatgcagccccatcttcatattcctactatgtgggcgagtatgagaagatgtacattagcctcttcttgcctctggtctacgagaacacgcgttccatctcatactcccccagcagcacaaccgaaggctacctgtacattgacctttctgcccctgtcccaatggctgaacgttacgacaacactacctccggctcatactacggcgatacagaccactacgactacgacactagcgtggcgtttgactacggttcctatccggtaggccgctttgccaacgaattcggcttccacagcatgcccagcctccagacatggcaacaagctgtcgacactgaggatctttacttcaacagcagcgtcgtcatgctgcgcaaccaccacgatcccgcaggtggtctcatgacggacaactacgcgaactcggccactggcatgggcgaaatgaccatgggcgtggtaagctactatccgataccgagtaaatccgaccacatctccaacttcagcgcctggtgccatgccacccagctctttcaggcagacatgtacaaaagtcagatccagttctaccgtcgtggaagtggcatgcccgagcgccagcttggctccttgtattggcagctcgaagatatctggcaagcgccatcatgggcaggcattgagtacggtggtagatggaaggtccttcaccacgttatgagagatatctatcagcctgttattgtttcacctttttggaactatactaccggctcgttggatgtctatgttacttccgatctgtggagccctgcagcaggtactgtcgacttgacctggttggacctgtccggccgccctattgcgggtaacgcgggcacgccaaaatctgttccctttaccgtgggaggtctcaacagcactcgcatctatgggacgaatgtttcttctctgggcttgccggatactaaagatgctgttctgatcctctcgctctcggctcacggccgtcttccgaactcagaccggaccaccaacttgactcatgagaattacgctacgctttcttggcccaaggatttgaagattgttgacccgggacttaagataggacacagctcaaagaagacaaccgttacggtggaagctacatccggtgtttcattgtacacctggctcgactacccagagggtgtggtgggatactttgaagagaatgccttcgtcttagcaccaggcgagaagaaagagattagttttactgttctagaggacactactgacggggcttgggtccgtaacatcaccgtccagagtctctgggaccaaaaggttcgcggttga

SEQ ID NO: 70 - protein, 931 aa, Aspergillus niger
MRHSIGLAAALLAPTLPVALGQYIRDLSTEKWTLSSRALNRTVPAQFPSQVHLDLLRAGVIGEYHGLNDFNLRWIAAANWTYTSQPIKGLLDNYDSTWLVFDGLDTFATISFCGQQIASTDNQFRQYAFDVSTALGSCKGDPVLSINFGSAPNIVDAIAQDSNSQKWPDDVQLTYEYPNRWFMRKEQSDFGWDWGPAFAPAGPWKPAYIVQLDKKESVYVLNTDLDIYRKGQINYLPPDQSQPWVVNASIDILGPLPTKPTMSIEVRDTHSGTILTSRTLNNVSVAGNAITGVTVLDGLTPKLWWPQGLGDQNLYNVSITVQSRGNQTVASVNKRTGFRTIFLNQRNITEAQRAQGIAPGANWHFEVNGHEFYAKGSNLIPPDSFWTRVTEEKMSRLFDAVVVGNQNMLRVWSSGAYLHDYIYDLADEKGILLWSEFEFSDALYPSDDAFLENVAAEIVYNVRRVNHHPSLALWAGGNEIESLMLPRVKDAAPSSYSYYVGEYEKMYISLFLPLVYENTRSISYSPSSTTEGYLYIDLSAPVPMAERYDNTTSGSYYGDTDHYDYDTSVAFDYGSYPVGRFANEFGFHSMPSLQTWQQAVDTEDLYFNSSVVMLRNHHDPAGGLMTDNYANSATGMGEMTMGVVSYYPIPSKSDHISNFSAWCHATQLFQADMYKSQIQFYRRGSGMPERQLGSLYWQLEDIWQAPSWAGIEYGGRWKVLHHVMRDIYQPVIVSPFWNYTTGSLDVYVTSDLWSPAAGTVDLTWLDLSGRPIAGNAGTPKSVPFTVGGLNSTRIYGTNVSSLGLPDTKDAVLILSLSAHGRLPNSDRTTNLTHENYATLSWPKDLKIVDPGLKIGHSSKKTTVTVEATSGVSLYTWLDYPEGVVGYFEENAFVLAPGEKKEISFTVLEDTTDGAWVRNITVQSLWDQKVRG

SEQ ID NO: 71 - DNA, 10207 bp, Artificial sequence
  Plasmid pQM27
gcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattgtttaaacgtcgaccgaatgtaggattgttatccgaactctgctcgtagaggcatgttgtgaatctgtgtcgggcaggacacgcctcgaaggttcacggcaagggaaaccaccgatagcagtgtctagtagcaacctgtaaagccgcaatgcagcatcactggaaaatacaaaccaatggctaaaagtacataagttaatgcctaaagaagtcatataccagcggctaataattgtacaatcaagtggctaaacgtaccgtaatttgccaacggcttgtggggttgcagaagcaacggcaaagccccacttccccacgtttgtttcttcactcagtccaatctcagctggtgatcccccaattgggtcgcttgtttgttccggtgaagtgaaagaagacagaggtaagaatgtctgactcggagcgttttgcatacaaccaagggcagtgatggaagacagtgaaatgttgacattcaaggagtatttagccagggatgcttgagtgtatcgtgtaaggaggtttgtctgccgatacgacgaatactgtatagtcacttctgatgaagtggtccatattgaaatgtaagtcggcactgaacaggcaaaagattgagttgaaactgcctaagatctcgggccctcgggccttcggcctttgggtgtacatgtttgtgctccgggcaaatgcaaagtgtggtaggatcgaacacactgctgcctttaccaagcagctgagggtatgtgataggcaaatgttcaggggccactgcatggtttcgaatagaaagagaagcttagccaagaacaatagccgataaagatagcctcattaaacggaatgagctagtaggcaaagtcagcgaatgtgtatatataaaggttcgaggtccgtgcctccctcatgctctccccatctactcatcaactcagatcctccaggagacttgtacaccatcttttgaggcacagaaacccaatagtcaaccgcggactgcgcaccatgcgccacagcatcggattggcagcggccctactggcaccaaccctacctgtagcgttgggtcaatatattcgagacttaagcaccgagaaatggactctcagtagtcgagccttgaatcggacagtacctgctcaatttccatcgcaggttcacttagatctactaagggccggagtgattggtgagtactatttggaagtcaagtcctgtgagtatacaaccgctaacagcctcaatagatgatccgtaagtgacttcatctgccatggatgagaattgaatcgcactaaatattgctggagataccatggtttgaacgatttcaatcttcgctggatcgctgctgccaactggacttataccagtcaacccatcaaaggcctgtgagtcgctgtgaagtttgtgcaatgtcgttcgacaatactaagaaccaatagcctggacaattacgactcaacttggctcgtgtttgacggactggacactttcgcaacaatctcattctgtgggcagcaaatcgcatccacggacaatcagtttcgccagtatgcgttcgatgtatccaccgcactagggtcctgcaaaggagatcctgttctgagcatcaactttggaagcgcaccgaatattgttgatgctatcgcacaggactctaattcgcaaagtaagtttcagaggtgggggactgccgaagttgttacatgctaattgtatatagaatggcccgatgacgtccaactcacctacgagtacccaaatcggtggtttatgcgcaaagaacaatcggacttcggatgggattggggtccagcatttgcccctgcaggtccatggaagcctgcatatattgttcagctagacaagaaagaaagtgtctatgtcctgaacacggatttggatatataccgaaagggccaaattaactaccttccgccagaccagagccaaccttgggtcgtcaacgctagcattgacattttgggtccactacctaccaaaccaaccatgtcgattgaagtgcgcgatactcattctggcacgattcttacttcgcggactctgaacaatgtcagtgtggctggtaatgccataactggtgtcaccgttctcgacgggctgaccccgaaactgtggtggccgcaaggcctcggtgatcagaacctctacaatgtttctatcactgtccaaagtagaggaaaccagaccgtggccagtgtgaacaaacggacgggcttccgcaccatttttctcaaccagcgcaacattactgaagcacagcgtgcgcaaggaatcgcccctggagcaaactggcactttgaagtcaacggtcatgagttctacgcaaaaggatcgaaccttatcccaccagacagtttctggacccgtgttacagaagagaagatgtcacggctattcgatgcagtggtcgttggaaaccagaatatgctccgtgtctggtcctccggcgcgtacctgcatgactacatctatgatctggccgatgaaaagggcattctcttatggagcgagttcgagttcagtgacgctttatatccctccgacgacgctttcctcgagaacgttgctgctgagatagtatacaatgttcgacgagtgaaccaccatccctccttggctctatgggctggcggaaatgaaatcgaatccttgatgctcccacgtgtcaaagatgcagccccatcttcatattcctactatgtgggcgagtatgagaagatgtacattagcctcttcttgcctctggtctacgagaacacgcgttccatctcatactcccccagcagcacaaccgaaggctacctgtacattgacctttctgcccctgtcccaatggctgaacgttacgacaacactacctccggctcatactacggcgatacagaccactacgactacgacactagcgtggcgtttgactacggttcctatccggtaggccgctttgccaacgaattcggcttccacagcatgcccagcctccagacatggcaacaagctgtcgacactgaggatctttacttcaacagcagcgtcgtcatgctgcgcaaccaccacgatcccgcaggtggtctcatgacggacaactacgcgaactcggccactggcatgggcgaaatgaccatgggcgtggtaagctactatccgataccgagtaaatccgaccacatctccaacttcagcgcctggtgccatgccacccagctctttcaggcagacatgtacaaaagtcagatccagttctaccgtcgtggaagtggcatgcccgagcgccagcttggctccttgtattggcagctcgaagatatctggcaagcgccatcatgggcaggcattgagtacggtggtagatggaaggtccttcaccacgttatgagagatatctatcagcctgttattgtttcacctttttggaactatactaccggctcgttggatgtctatgttacttccgatctgtggagccctgcagcaggtactgtcgacttgacctggttggacctgtccggccgccctattgcgggtaacgcgggcacgccaaaatctgttccctttaccgtgggaggtctcaacagcactcgcatctatgggacgaatgtttcttctctgggcttgccggatactaaagatgctgttctgatcctctcgctctcggctcacggccgtcttccgaactcagaccggaccaccaacttgactcatgagaattacgctacgctttcttggcccaaggatttgaagattgttgacccgggacttaagataggacacagctcaaagaagacaaccgttacggtggaagctacatccggtgtttcattgtacacctggctcgactacccagagggtgtggtgggatactttgaagagaatgccttcgtcttagcaccaggcgagaagaaagagattagttttactgttctagaggacactactgacggggcttgggtccgtaacatcaccgtccagagtctctgggaccaaaaggttcgcggttgattaattaagctccgtggcgaaagcctgacgcaccggtagattcttggtgagcccgtatcatgacggcggcgggagctacatggccccgggtgatttattttttttgtatctacttctgacccttttcaaatatacggtcaactcatctttcactggagatgcggcctgcttggtattgcgatgttgtcagcttggcaaattgtggctttcgaaaacacaaaacgattccttagtagccatgcattttaagataacggaatagaagaaagaggaaattaaaaaaaaaaaaaaaacaaacatcccgttcataacccgtagaatcgccgctcttcgtgtatcccagtaccacggcaaaggtatttcatgatcgttcaatgttgatattgttcccgccagtatggctccacccccatctccgcgaatctcctcttctcgaacgcggtagtggcgcgccaattggtaatgacccatagggagacgaattaactagaggtgactgacacctggcggtagacaatcaatccatttcgctatagttaaaggatggggatgagggcaattggttatatgatcatgtatgtagtgggtgtgcataatagtagtgaaatggaagccaagtcatgtgattgtaatcgaccgacggaattgaggatatccggaaatacagacaccgtgaaagccatgctctttccttcgtgtagaagaccagacagacagtccctgatttacccttgcacaaagcactagaaaattagcattccatccttctctgcttgctctgctgatatcactgtcattcaatgcatctggaaacgcaaccctgaagggattcttcctttgagagatggaagcgtgtcatatctcttcggttctacggcaggtttttttctgctctttcgtagcatggcatggtcacttcagcgcttatttacagttgctggtattgatttcttgtgcaaattgctatctgacacttattagctatggagtcaccacatttcccagcaacttccccacttcctctgcaatcgccaacgtcctctcttcactgagtctccgtccgataacctgcactgcaaccggtgccccatgatacgcctccggatcatactcttcctgcacgagggcatcaagctcactaaccgccttgaaactctcattcttcttatcgatgttcttatccgcaaaggtaaccggaacaaccacgctcgtgaaatccagcaggttgatcacagaggcatacccatagtaccggaactggtcatgccgtaccgcagcggtaggcgtaatcggcgcgatgatggcgtccagttccttcccggccttttcttcagcctcccgccatttctcaaggtactccatctggtaattccacttctggagatgcgtgtcccagagctcgttcatgttaacagctttgatgttcgggttcagtaggtctttgatatttggagtcgccggctcgccggatgcactgatatcgcgcattacgtcggcgctgccgtcagccgcgtagatatgggagatgagatcgtggccgaaatcgtgcttgtatggcgtccacggggtcacggtgtgaccggctttggcgagtgcggcgacggtggtttccacgccgcgcaggataggagggtgtggaaggacattgccgtcgaagttgtagtagccgatattgagcccgccgttcttgatcttggaggcaataatgtccgactcggactggcgccagggcatggggatgaccttggagtcgtatttccaaggctcctgaccgaggacggatttggtgaagaggcggaggtctaacatacttcatcagtgactgccggtctcgtatatagtataaaaagcaagaaaggaggacagtggaggcctggtatagagcaggaaaagaaggaagaggcgaaggactcaccctcaacagagtgcgtaatcggcccgacaacgctgtgcaccgtctcctgaccctccatgctgttcgccatctttgcatacggcagccgcccatgactcggccttagaccgtacaggaagttgaacgcggccggcactcgaatcgagccaccgatatccgttcctacaccgatgacgccaccacgaatcccaacgatcgcaccctcaccaccagaactgccgccgcacgaccagttcttgttgcgtgggttgacggtgcgcccgatgatgttgttgactgtctcgcagaccatcagggtctgcgggacagaggtcttgacgtagaagacggcaccggctttgcggagcatggttgtcagaaccgagtccccttcgtcgtacttgtttagccatgagatgtagcccattgatgtttcgtagccctggtggcatatgttagctgacaaaaagggacatctaacgacttaggggcaacggtgtaccttgactcgaagctggtctttgagagagatggggaggccatgaagtggaccaacgggtctcttgtgctttgcgtagtattcatcgagttcccttgcctgcgcgagagcggcgtcagggaagaactcgtgggcgcagtttgtctgcacagaagccagcgtcagcttgatagtcccataaggtggcgttgttacatctccctgagaggtagaggggaccctactaactgctgggcgattgctgcccgtttacagaatgctagcgtaacttccaccgaggtcaactctccggccgccagcttggacacaagatctgcagcggaggcctctgtgatcttcagttcggcctctgaaaggatccccgatttctttgggaaatcaataacgctgtcttccgcaggcagcgtctggactttccattcatcagggatggtttttgcgaggcgggcgcgcttatcagcggccagttcttcccaggattgaggcattctgtgttagcttatagtcaggatgttggctcgacgagtgtaaactgggagttggcatgagggttatgtaggcttctttagccccgcatccccctcattctcctcattgatcccgggggagcggatggtgttgataagagactaattatagggtttagctggtgcctagctggtgattggctggcttcgccgaattttacgggccaaggaaagctgcagaaccgcggcactggtaaacggtaattaagctatcagccccatgctaacgagtttaaattacgtgtattgctgataaacaccaacagagctttactgaaagatgggagtcacggtgtggcttccccactgcgattattgcacaagcagcgagggcgaacttgactgtcgtcgctgagcagcctgcagtcaaacatacatatatatcaaccgcgaagacgtctggccttgtagaacacgacgctccctagcaacacctgccgtgtcagcctctacggttgttacttgcattcaggatgctctccagcgggcgagctattcaaaatattcaaagcaggtatctcgtattgccaggattcagctgaagcaacaggtgccaaggaaatctgcgtcggttctcatctgggcttgctcggtcctggcgtagaatgcatcctagagtttaaacagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaacggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagc

SEQ ID NO: 72 - DNA, 46 bp, Artificial sequence
  Primer 1210956
tgattacgaattgtttaaacggatccgaatgtaggattgttatccg

SEQ ID NO: 73 - DNA, 72 bp, Artificial sequence
  Primer 1201606
gaagttcctatactttctagagaataggaactcggaataggaacttcaaccttatgggactatcaagctgac

SEQ ID NO: 74 - DNA, 50 bp, Artificial sequence
  Primer 1210957
gttacattgacgtacttataagaagttcctatactttctagagaatagga

SEQ ID NO: 75 - DNA, 357 bp, Artificial sequence
  T. reesei cbh1 5′ probe sequence
tagggtcggcaacggcaaaaaagcacgtggctcaccgaaaagcaagatgtttgcgatctaacatccaggaacctggatacatccatcatcacgcacgaccactttgatctgctgtaaactcgtattcgccctaaaccgaagtgcgtggtaaatctacacgtgggcccctttcggtatactgcgtgtgtcttctctaggtgccattcttttcccttcctctagtgttgaattgtttgtgttggagtccgagctgtaactacctctgaatctctggagaatggtggactaacgactaccgtgcacctgcatcatgtatataatagtgatcctgagaaggggggtttggagcaatgtggg

SEQ ID NO: 76 - DNA, 87 bp, Artificial sequence
  Primer 1205503
gggagatgtaacaacgccttgaagttcctattccgagttcctattcttcaaatagtataggaacttcaaccttatgggactatcaag

SEQ ID NO: 77 - DNA, 87 bp, Artificial sequence
  Primer 1205504
cttgatagtcccataaggttgaagttcctatactatttgaagaataggaactcggaataggaacttcaaggcgttgttacatctccc

SEQ ID NO: 78 - DNA, 84 bp, Artificial sequence
  Primer 1205505
gcccctaagtcgttagatgtttgaagttcctattccgagttcctattcttcaaatagtataggaacttcaccctttttgtcagc

SEQ ID NO: 79 - DNA, 84 bp, Artificial sequence
  Primer 1205506
gctgacaaaaagggtgaagttcctatactatttgaagaataggaactcggaataggaacttcaaacatctaacgacttaggggc

SEQ ID NO: 80 - DNA, 83 bp, Artificial sequence
  Primer 1205507
ctataccaggcctccacttgaagttcctattccgagttcctattcttcaaatagtataggaacttcatgtcctcctttcttgc

SEQ ID NO: 81 - DNA, 83 bp, Artificial sequence
  Primer 1205508
gcaagaaaggaggacatgaagttcctatactatttgaagaataggaactcggaataggaacttcaagtggaggcctggtatag